(19) United States
(12) Patent Application Publication
(10) Pub. No.: US 2020/0080147 A1
(43) Pub. Date: Mar. 12, 2020

(54) METHODS AND COMPOSITIONS FOR TUMOR ASSESSMENT

(71) Applicant: PLURISTEM LTD., Haifa (IL)

(72) Inventors: Zami ABERMAN, Tel-Mond (IL); Rachel OFIR, Adi (IL); Hoshea Yissachar ALLEN, Bet Shemesh (IL)

(73) Assignee: PLURISTEM LTD., Haifa (IL)

(21) Appl. No.: 16/485,160

(22) PCT Filed: Feb. 18, 2018
(86) PCT No.: PCT/IB2018/050984

§ 371 Date: Aug. 11, 2019

Related U.S. Application Data
(60) Provisional application No. 62/460,890, filed on Feb. 20, 2017.

(51) Int. Cl. C12Q 1/6881

(52) U.S. Cl. CPC C12Q 1/6881; C12Q 2600/156; C12Q 2600/106

(57) ABSTRACT

Described herein are methods and articles of manufacture for determining the suitability of a tumor or neoplastic cell to treatment with adherent stromal cells or with conditioned medium derived therefrom.

### CROSS-REFERENCE TO RELATED APPLICATIONS

This application claims priority from U.S. Provisional Patent Appl. No. 62/460,890, filed Feb. 20, 2017, which is hereby incorporated by reference in its entirety.

### FIELD

Described herein are methods and articles of manufacture for determining the suitability of a tumor or neoplastic cell to cell-based therapy.

### BACKGROUND

Cell therapy is beginning to be used for tumor treatment. However, it is difficult to predict empirically which tumors will be sensitive to a particular type of cell therapy. The present disclosure is intended to address this deficiency.

### SUMMARY

Adherent stromal cells (ASC) are demonstrated herein to be useful for treatment, prevention, and inhibition of growth of cancers, tumors, and neoplasms. Aspects of the disclosure relate to the discovery of genes (referred to herein as “ASC-susceptibility genes”) the mutational status of which is informative of the extent to which a subject is susceptible to treatment with ASCs. In some embodiments, an informative mutation indicates that the subject will be non-responsive to treatment with ASCs. However, in some embodiments, an informative mutation indicates that the subject will be responsive to treatment with ASCs. Accordingly, in some embodiments, methods are provided herein for treating subjects having a treatment informative mutation in an ASC-susceptibility gene that indicates the subject will be will be responsive to treatment with ASCs. In some embodiments, the methods involve administering to such subjects an effective amount of ASC. In some embodiments, described herein are methods and articles of manufacture for determining the extent to which tumors and neoplastic cells are susceptible to treatment with ASC or with conditioned medium derived therefrom, based on the mutational status of ASC-susceptibility genes.

In certain embodiments, the described ASC have been prepared by culturing in 2-dimensional (2D) culture, 3-dimensional (3D) culture, or a combination thereof. Non-limiting examples of 2D and 3D culture conditions are provided in the Detailed Description and in the Examples. Alternatively or in addition, the cells have been treated with pro-inflammatory cytokines; and/or are a placental cell preparation. In certain embodiments, the placental cell preparation is predominantly fetal cells; predominantly maternal cells; or a mixture of fetal cells and maternal cells, which is, in more specific embodiments, enriched for fetal cells or enriched for maternal cells. The term “ASC”, except where indicated otherwise, may refer, in various embodiments, to adherent stromal cells either before or after incubation with pro-inflammatory cytokines.

Alternatively or in addition, the cells are mesenchymal-like ASC, which exhibit a marker pattern similar to mesenchymal stromal cells, but do not differentiate into osteocytes, under conditions where “classical” mesenchymal stem cells (MSC) would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where mesenchymal stem cells would differentiate into osteocytes or adipocytes, respectively. The MSC used for comparison in these assays are, in some embodiments, MSC that have been harvested from bone marrow (BM) and cultured under 2D conditions. In other embodiments, the MSC used for comparison have been harvested from BM and cultured under 2D conditions, followed by 3D conditions.

In various embodiments, the described ASC are able to exert the described therapeutic effects, each of which is considered a separate embodiment, with or without the ASC themselves engrafting in the host. For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days; or the cells survive for more than 3 days, more than 4 days, more than 5 days, more than 6 days, more than 7 days, more than 8 days, more than 9 days, more than 10 days, or more than 14 days.

Reference herein to “growth” of a population of cells is intended to be synonymous with expansion of a cell population.

Reference herein to a “gene” includes any nucleotide sequence that encodes a functional RNA or protein product. In some embodiments, a gene is transcribed in at least one type of eukaryotic cell, whether or not the transcript is used to produce a protein product.

Reference herein to genes is intended to encompass homologues of the genes within a species (paralogs) and across different species (orthologs), for example, where an animal tumor is tested for susceptibility to treatment with ASC or CM.

Except where otherwise indicated, all ranges mentioned herein are inclusive.

Except where otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIGS. 2A-B are graphs depicting secretion, measured by fluorescence, of various factors following incubation of ASC with TNF-alpha+IFN-gamma (unfilled bars) or control media (filled bars) in two separate experiments. C-D are graphs depicting fold-increase of secretion, measured by fluorescence, of GRO, IL-8, MCP-1, and RANTES (C), and IL-6, MCP-3, Angiogenin, Insulin-like Growth Factor Binding Protein-2 (IGFBP-2), Osteopontin, and Osteoprotegerin (D) following incubation of ASC with TNF-alpha alone, relative to incubation with control media (no cytokines).

FIGS. 3A-B are graphs depicting fold-increase relative to control medium (containing no cytokines) in secretion of MCP-1 (A) and GM-CSF (B) in several experiments, as measured by ELISA.

FIGS. 4A-B are graphs depicting secretion of various factors by TNF-alpha+IFN-gamma (A) or TNF-alpha alone (B) in the presence or absence of FBS. In (A), gray, white, and black bars indicate TNF-alpha+IFN-gamma; TNF-alpha+IFN-gamma+FBS; and control (no cytokines or serum), respectively. In (B), gray, white, and black bars indicate TNF-alpha alone; TNF-alpha+FBS; and control (no cytokines or serum), respectively.

FIG. 5 is a graph showing expression of RANTES (CCL5) in the following samples, ordered from left to right: placental cells not treated with cytokines (first 7 bars from left) or treated with TNF-alpha, IFN-gamma, or TNF-alpha+IFN-gamma (bars 8-11, 12-14, and 15-22 from left, respectively). The expression level of a representative sample in the TNF-alpha+IFN-gamma group was arbitrarily assigned a value of 1.

FIGS. 6A-C are bar graphs showing the effect of the highest concentration of each of the 4 tested ASC CM on 3 renal cell carcinoma cell lines, namely 769-P (A), 786-O (B), and ACHN (C). The dose dependence of group 1 for 769-P and 786-O are depicted in D-E, respectively.

FIG. 7 contains bar graphs showing the effect of the highest concentration of each of the 4 tested ASC CM on 4 hepatocellular carcinoma lines, namely Hep 3B (A), Hep G2 (C), SNU-449 (E), and C3A (G). The dose dependence of group 1 for Hep 3B, Hep G2, and SNU-449 are depicted in B, D, and F, respectively.

FIG. 8A is a bar graph showing the effect of the highest concentration of each of the 4 tested ASC CM on the breast adenocarcinoma line MDA-MB-231. The dose dependence of group 1 for this line is depicted in B.

FIG. 8C is a bar graph showing the effect of the highest concentration of each of the 4 tested ASC CM on the breast carcinoma line HCC-1395. The dose dependence of group 1 for this line is depicted in D.

FIG. 9A is a bar graph showing the effect of the highest concentration of each of the 4 tested ASC CM on the lung adenocarcinoma line NCI-H1792. The dose dependence of group 1 for this line is depicted in B.

FIG. 10A is a bar graph showing the effect of the highest concentration of each of the 4 tested ASC CM on the rhabdomyosarcoma line RD. The dose dependence of group 1 for this line is depicted in B.

FIG. 11A is a graphical representation of the scores for each profiled gene for the breast cancer cell lines marker gene analysis. B is centroid plot showing the mean expression value for the 5 breast cancer cell lines for all of the genes downregulated (scores <−5) in the responsive breast cell lines. The 2 responsive breast cancer cell lines (HCC-1395 and MDA-MB-231) are shown on the left, and the other 3 breast cancer cell lines (BT474, MCF7 and T47D) are shown on the right. The error bars represent the standard deviation.

FIGS. 12A-B are tables summarizing the genes in the MHC Class I antigen processing and presentation pathway (A) and the cytokine signaling pathway (B) that are downregulated and/or exclusively mutated in each of the responsive cell lines.

FIG. 13A is a plot of p-value (vertical axis) vs. log effect (horizontal axis) of mutated genes positively and negatively correlated (p-value <0.05) with responsiveness to ASC treatment. Positive correlation is indicated by a positive log effect, while negative correlation is indicated by a negative log effect. B-C are charts setting forth the specific mutations found in the genes that were negatively (B) and positively (C) correlated with responsiveness. Transcript numbers are Ensemble numbers, depicted without the ENST and the preceding zeros. Substit., insert., and delet. denote substitution, insertion, and deletion, respectively.

FIG. 14 is a heat map showing expression of 305 classifier genes useful for characterizing breast cancer lines as Luminal, Basal A, or Basal B by hierarchical clustering as per Neve et al. Red and green depict upregulated and downregulated genes, respectively.

FIG. 15A is a classification tree corresponding to a close-up view of the top of FIG. 14, and showing which breast cancer cell lines were characterized for TRAIL sensitivity and ASC sensitivity. The figure also incorporates data from Rahman et al. Asterisks denote breast cell lines tested for TRAIL sensitivity, where black and red denote TRAIL insensitive and TRAIL-sensitive, respectively. Blue reverse-highlighting denotes lines that were tested for TRAIL sensitivity and are TN. Enclosure in a black box denotes lines that were tested for both ASC sensitivity and TRAIL sensitivity. B depicts the data from tested breast cancer cell lines from A in tabular form, and also includes information on clinical sub-type, namely whether or not the cell lines are ER positive, PR positive, or Her2/neu amplified.

FIG. 16A is a heat map showing expression of 169 probe sets used for another hierarchical clustering, using data from the Cancer Cell Line Encyclopedia (CCLE). FIG. 16B is a classification tree corresponding to a close-up view of the top of A, and showing which breast cancer cell lines were characterized for ASC sensitivity. C depicts the data from tested breast cancer cell lines from B in tabular form, and also includes information on clinical sub-type, namely whether or not the cell lines are ER positive, PR positive, or Her2/neu amplified. Cell lines that grouped differently from the previous analysis are circled in B and indicated by asterisks in C.

FIG. 17 is a listing (right side) of the pathways in which classifer genes in the 3 sections of the heatmap (shown on the left side) (of the hierarchical clustering analysis by Neve et al) participate.

FIG. 18 is a boxplot showing the correlation between the TRAIL sensitivity and ASC sensitivity of the cells lines tested herein. The minimum, first quartile, median, mean, third quartile and maximum values are depicted. The heavy line inside each box indicates the mean, and the lighter line inside the box indicates the median.

FIG. 19A is a bar graph showing the mean volume (mm³) of implanted tumors in mice untreated or treated with ASC IM or IV (first, second and third bars from left, respectively). Left, middle, and right bars in each series are the control, IM, and IV groups, respectively. Left, center, and right datasets depict tumor sizes at days 5, 7, and 9, respectively. B is a bar graph showing average tumor sizes from each timepoint for IV-injected mice, and C is a plot showing the same data. D is a bar graph showing average tumor sizes from each timepoint for IM-injected mice, and E is a plot showing the same data.

FIG. 20A is a perspective view of a carrier (or “3D body”), according to an exemplary embodiment. B is a perspective view of a carrier, according to another exemplary embodiment. C is a cross-sectional view of a carrier, according to an exemplary embodiment.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention disclosed herein relate to the discovery of a set of adherent stromal cell (ASC)-susceptibility genes.

In certain embodiments, there is provided a method of determining the susceptibility of a tumor or neoplastic cell to treatment with adherent stromal cells (ASC), the method comprising testing the tumor or neoplastic cell for a mutation in an ASC-susceptibility gene selected from the group consisting of:

a. TAF1, ZNF248, and DPY19L4A, where the presence of a mutation indicates susceptibility to treatment with ASC; and

b. ZNF708, PRG4, CTU2, GOLGA8A, PTCH2, NSD1, QRICH2, SPAG5, C6orf165, LIMK2, EIF4B, LATS1, SCN8A, VPS8, KIAA1161, AFF3, KIAA1715, SLC6A17, SF1, KIAA0494, ZNF592, and BAZ2B, where the presence of a mutation indicates lack of susceptibility to treatment with ASC.

In other embodiments, there is provided a method of treating a subject having a tumor or neoplastic cell, the method comprising administering to the subject an effective amount of ASC, wherein the subject was selected for the treatment based on the presence or absence of an ASC treatment informative mutation in an ASC-susceptibility gene selected from:

a. an ASC sensitivity gene, wherein the presence of the ASC treatment informative mutation indicates that the subject will be responsive to treatment with ASC; and

b. an ASC resistance gene, wherein the presence of the ASC treatment informative mutation indicates that the subject will be non-responsive to treatment with ASC.

In still other embodiments, there is provided a method for evaluating a subject having a tumor, the method comprising:

a. obtaining, from cells of the subject, nucleic acids that comprise one or more sequences of one or more ASC-susceptibility genes selected from:

i. ASC-sensitivity genes, and

ii. ASC resistance genes; and

b. performing a sequencing procedure to detect an ASC treatment informative mutation in the one or more sequences of the one or more genes,

wherein:
for an ASC-sensitivity gene, the presence of the ASC treatment informative mutation indicates that the subject will be responsive to treatment with ASC; and
for an ASC-resistance gene, the presence of the ASC treatment informative mutation indicates that the subject will be non-responsive to treatment with ASC.

In yet other embodiments, there is provided a method for treating a subject having a tumor, the method comprising:

a. obtaining, from cells of the subject, nucleic acids that comprise one or more sequences of one or more ASC-susceptibility genes selected from;

i. ASC-sensitivity genes; and

ii. ASC-resistance genes; and

b. performing a sequencing procedure to detect an ASC treatment informative mutation in the one or more sequences of the one or more ASC-susceptibility genes; and

c. treating the subject with an effective amount of ASCs after detecting the presence or absence of a treatment informative mutation;

wherein:
for an ASC-sensitivity gene, the presence of the ASC treatment informative mutation indicates that the subject will be responsive to treatment with ASC; and
for an ASC-resistance gene, the presence of the ASC treatment informative mutation indicates that the subject will be non-responsive to treatment with ASC.

In other embodiments is provided an article of manufacture for determining the susceptibility of a tumor or neoplastic cell to treatment with ASC, the article comprising a means of testing the tumor or neoplastic cell for a mutation in an ASC-susceptibility gene selected from the group consisting of:

a. TAF1, ZNF248, and DPY19L4, where the presence of a mutation indicates susceptibility to treatment with ASC; and

b. ZNF708, PRG4, CTU2, GOLGA8A, PTCH2, NSD1, QRICH2, SPAG5, C6orf165, LIMK2, EIF4B, LATS1, SCN8A, VPS8, KIAA1161, AFF3, KIAA1715, SLC6A17, SF1, KIAA0494, ZNF592, and BAZ2B, where the presence of a mutation indicates lack of susceptibility to treatment with ASC.

As described herein, an ASC-susceptibility gene is a gene whose mutational status is informative of the extent to which a subject is susceptible to treatment with ASCs. ASC-susceptibility genes are classified as either ASC-sensitivity genes or ASC-resistance genes. Provided herein are exemplary ASC-susceptibility genes. Other ASC-susceptibility genes identified using the methods disclosed herein may also be used to determine whether a subject is susceptible to treatment with ASCs.

In some embodiments, the aforementioned article of manufacture is a kit. In other embodiments, the article is any other composition comprising a means for detecting mutations in the described genes.

In other embodiments is provided a method of determining the susceptibility of a tumor or neoplastic cell to treatment with conditioned medium (CM) derived from ASC, the method comprising testing the tumor or neoplastic cell for a mutation in a gene selected from the group consisting of:

a. TAF1, ZNF248, and DPY19L4, where the presence of a mutation indicates susceptibility to treatment with ASC; and

b. ZNF708, PRG4, CTU2, GOLGA8A, PTCH2, NSD1, QRICH2, SPAG5, C6orf165, LIMK2, EIF4B, LATS1, SCN8A, VPS8, KIAA1161, AFF3, KIAA1715, SLC6A17, SF1, KIAA0494, ZNF592, and BAZ2B, where the presence of a mutation indicates lack of susceptibility to treatment with ASC.

In other embodiments is provided an article of manufacture of determining the susceptibility of a tumor or neoplastic cell to treatment with CM derived from ASC, the article comprising a means for testing the tumor or neoplastic cell for a mutation in a gene selected from the group consisting of:

a. TAF1, ZNF248, and DPY19L4, where the presence of a mutation indicates susceptibility to treatment with ASC; and

b. ZNF708, PRG4, CTU2, GOLGA8A, PTCH2, NSD1, QRICH2, SPAG5, C6orf165, LIMK2, EIF4B, LATS1, SCN8A, VPS8, KIAA1161, AFF3, KIAA1715, SLC6A17, SF1, KIAA0494, ZNF592, and BAZ2B, where the presence of a mutation indicates lack of susceptibility to treatment with ASC.

In some embodiments, the aforementioned article of manufacture is a kit. In other embodiments, the article is any other composition comprising a means for detecting mutations in the described genes.

The aforementioned ASC may be derived from a placenta or, in other embodiments, from adipose tissue, or, in other embodiments, from other sources as described herein. As provided herein, administration of ASC is useful in treating neoplastic growths.

Except where indicated otherwise, susceptibility to treatment with ASC refers to treatment of cancer cells with whole, live ASC. In other embodiments, the cancer cells are treated with fractions of ASC, or with factors derived from ASC.

Except where indicated otherwise, susceptibility to treatment with conditioned medium (CM) refers to treatment of cancer cells with medium that has been incubated with ASC. In other embodiments, the cancer cells are treated with fractions of CM that has been incubated with ASC, or with factors derived from CM that has been incubated with ASC.

In certain embodiments, the mutation is in a gene selected from TAF1 (encodes Transcription initiation factor TFIID subunit 1; Uniprot accession no. P21675), ZNF248 (Uniprot accession no. Q8NDW4), and DPY19L4 (encodes Probable C-mannosyltransferase DPY19LA; Uniprot accession no. Q7Z388). As provided herein (Table 23), tumors with mutations in TAF1, ZNF248, and DPY19L4 are sensitive to treatment with ASC. Uniprot was accessed on Jan. 3, 2016 for the entries in this paragraph. In other embodiments, the tumor or neoplastic cell is tested for a mutation in at least two, or all three of the genes in this paragraph.

In other embodiments, the mutation is in a gene selected from ZNF708 (encodes Zinc finger protein 708; Uniprot accession no. P17019), PRG4 (encodes Proteoglycan 4; Uniprot accession no. Q92954), CTU2 (encodes Cytoplasmic tRNA 2-thiolation protein 2; Uniprot accession no. Q2VPK5), GOLGA8A (encodes Golgin subfamily A member 8A; Uniprot accession no. A7E2F4), PTCH2 (encodes Protein patched homolog 2; Uniprot accession no. Q9Y6C5), NSD1 (encodes Histone-lysine N-methyltransferase, H3 lysine-36 and H4 lysine-20 specific; Uniprot accession no. Q96L73), QRICH2 (encodes Glutamine-rich protein 2; Uniprot accession no. Q9H0J4), SPAG5 (encodes Sperm-associated antigen 5; Uniprot accession no. Q96R06), C6orf165 (Uniprot accession no. Q8IYR0), LIMK2 (encodes LIM domain kinase 2; Uniprot accession no. P53671), EIF4B (encodes Eukaryotic translation initiation factor 4B; Uniprot accession no. P23588), LATS1 (encodes Serine/threonine-protein kinase LATS1; Uniprot accession no. O95835), SCN8A (encodes Sodium channel protein type 8 subunit alpha; Uniprot accession no. Q9UQD0), VPS8 (encodes Vacuolar protein sorting-associated protein 8 homolog; Uniprot accession no. Q8N3P4), KIAA1161 (encodes Uncharacterized family 31 glucosidase KIAA1161; Uniprot accession no. Q6NSJ0), AFF3 (encodes AF4/FMR2 family member 3; Uniprot accession no. P51826), KIAA1715 (encodes Protein lunapark; Uniprot accession no. Q9C0E8), SLC6A17 (encodes Sodium-dependent neutral amino acid transporter SLC6A17; Uniprot accession no. Q9H1 V8), SF1 (encodes Splicing factor 1; Uniprot accession no. Q15637), KIAA0494 (encodes EF-hand calcium-binding domain-containing protein 14; Uniprot accession no. O75071), ZNF592 (encodes Zinc finger protein 592; Uniprot accession no. Q92610), and BAZ2B (encodes Bromodomain adjacent to zinc finger domain protein 2B; Uniprot accession no. Q9UIF8). As provided herein (Table 24), tumors with mutations in ZNF708, PRG4, CTU2, GOLGA8A, PTCH2, NSD1, QRICH2, SPAG5, C6orf165, LIMK2, EIF4B, LATS1, SCN8A, VPS8, KIAA1161, AFF3, KIAA1715, SLC6A17, SF1, KIAA0494, ZNF592, and BAZ2B are less likely to be responsive to treatment with ASC. Uniprot was accessed on Jan. 4, 2016, for the entries in this paragraph. In other embodiments, the tumor or neoplastic cell is tested for a mutation in at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the genes in this paragraph.

In certain embodiments of the aforementioned methods and articles of manufacture, the tumor or neoplastic cell is tested for a mutation in at least two of the described genes, in other embodiments between 2-20, 2-19, 2-18, 2-17, 2-16, 2-15, 2-14, 2-13, 2-12, 2-11, 2-10, 2-9, 2-8, 2-7, 2-6, 2-5, 2-4, or 2-3 of the genes. The genes may, in various embodiments, be selected from one or more of the aforementioned lists.

In other embodiments, the tumor or neoplastic cell is tested for a mutation in at least three of the genes, in other embodiments between 3-20, 3-19, 3-18, 3-17, 3-16, 3-15, 3-14, 3-13, 3-12, 3-11, 3-10, 3-9, 3-8, 3-7, 3-6, 3-5, or 3-4 of the genes. The genes may, in various embodiments, be selected from one or more of the aforementioned lists.

In still other embodiments, the tumor or neoplastic cell is tested for a mutation in at least four of the genes, in other embodiments between 4-20, 4-19, 4-18, 4-17, 4-16, 4-15, 4-14, 4-13, 4-12, 4-11, 4-10, 4-9, 4-8, 4-7, 4-6, or 4-5 of the genes. The genes may, in various embodiments, be selected from one or more of the aforementioned lists.

In other embodiments of the aforementioned methods and articles of manufacture, the tumor or neoplastic cell is also tested for (in addition to one or more of the aforementioned genes) a mutation selected from the group consisting of: SCN3A (encodes Sodium channel protein type 3 subunit alpha; Uniprot accession no. Q9NY46), DCHS1 (encodes Protocadherin-16; Uniprot accession no. Q96JQ0), PDGFRA (encodes Platelet-derived growth factor receptor alpha; Uniprot accession no. P16234), LGSN (encodes Lengsin; Uniprot accession no. Q5TDP6), EPHB4 (encodes Ephrin type-B receptor 4; Uniprot accession no. P54760), SEMA3E (encodes Semaphorin-3E; Uniprot accession no. O15041), EXTL3 (encodes Exostosin-like 3; Uniprot accession no. O43909), SFMBT1 (encodes Scm-like with four MBT domains protein 1; Uniprot accession no. Q9UHJ3), DUOX2 (encodes Dual oxidase 2; Uniprot accession no. Q9NRD8), CCDC137 (encodes Coiled-coil domain-containing protein 137; Uniprot accession no. Q6PK04), PCDH12 (encodes Protocadherin-12; Uniprot accession no. Q9NPG4), TLR1 (encodes Toll-like receptor 1; Uniprot accession no. Q15399), and GPR124 (encodes G-protein coupled receptor 124; Uniprot accession no. Q96PE1). As provided herein (Table 23), tumors with mutations in SCN3A, DCHS1, PDGFRA, LGSN, EPHB4, SEMA3E, EXTL3, SFMBT1, DUOX2, CCDC137, PCDH12, TLR, and GPR124 are sensitive to treatment with ASC. Uniprot was accessed on Jan. 4, 2016 for the entries in this paragraph. In other embodiments, the tumor or neoplastic cell is tested for a mutation in at least 2, at least 3, at least 4, at least 5, at least 6, at least 7, at least 8, at least 9, at least 10, at least 11, at least 12, or all 13 of the genes in this paragraph.

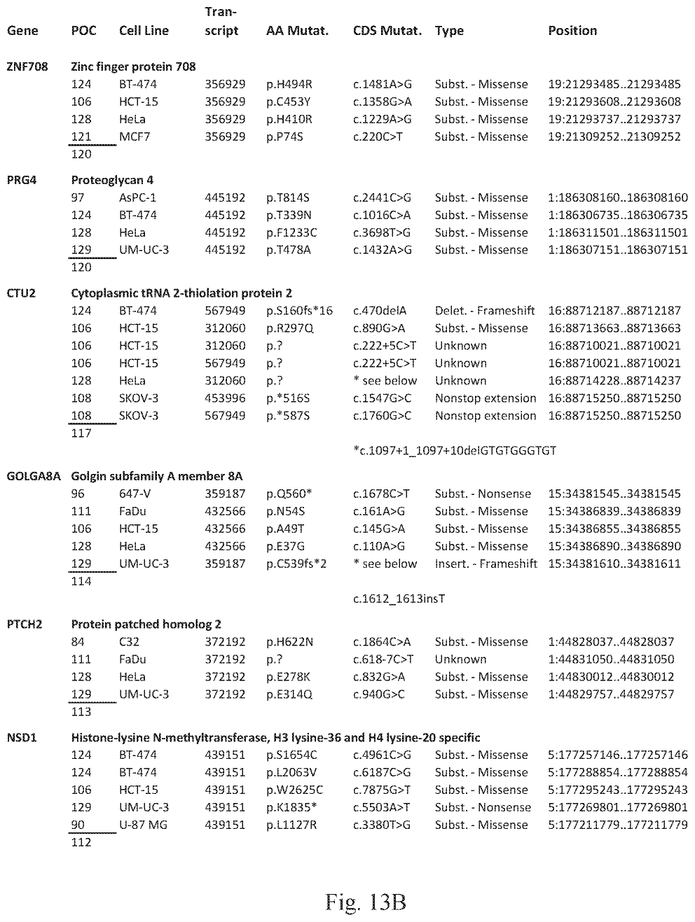

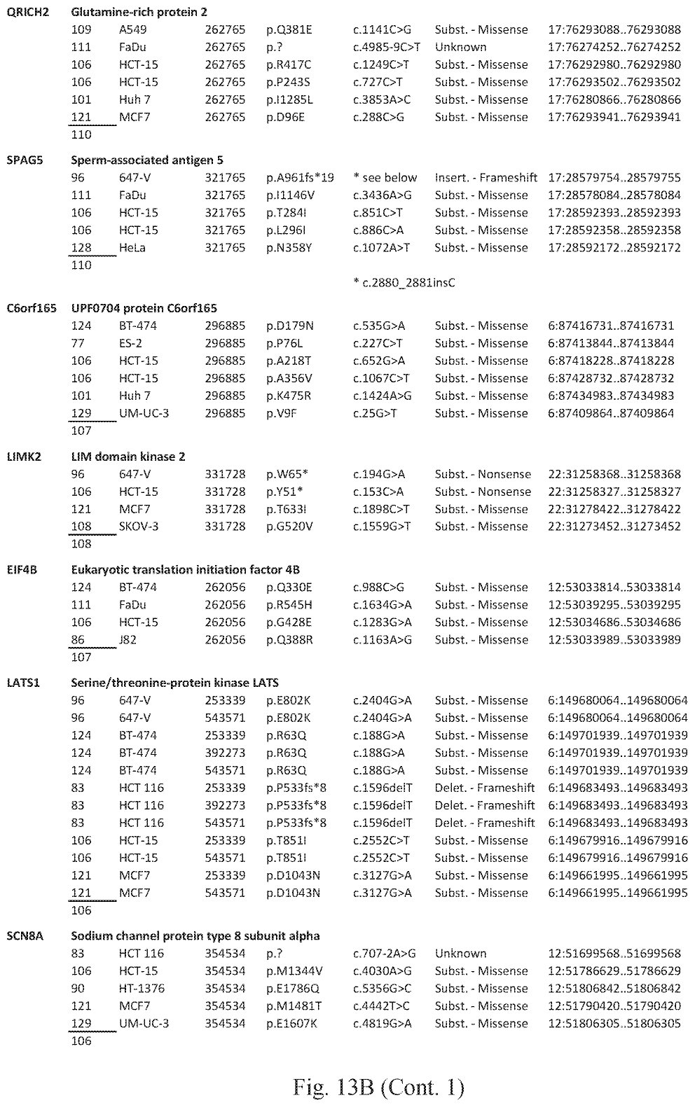

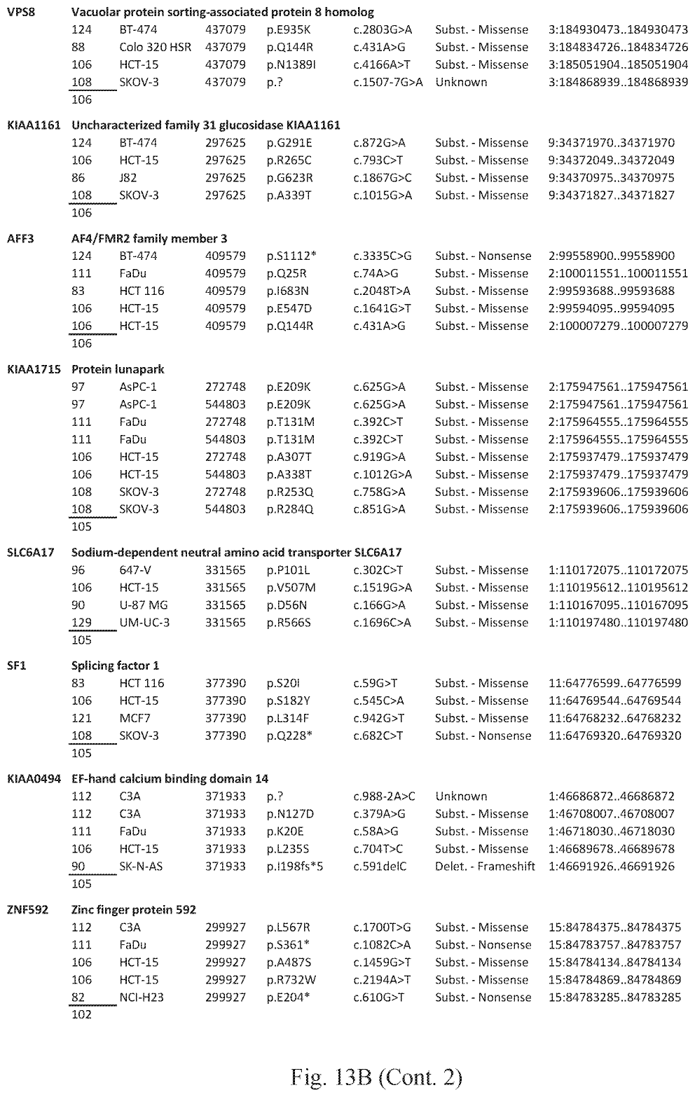

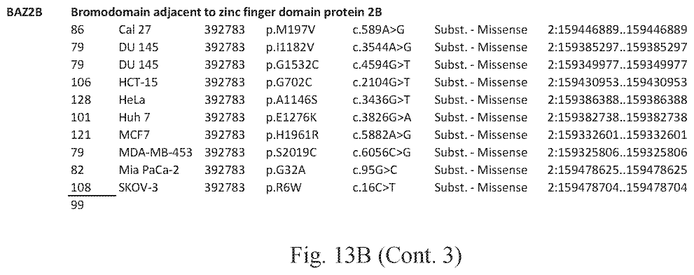

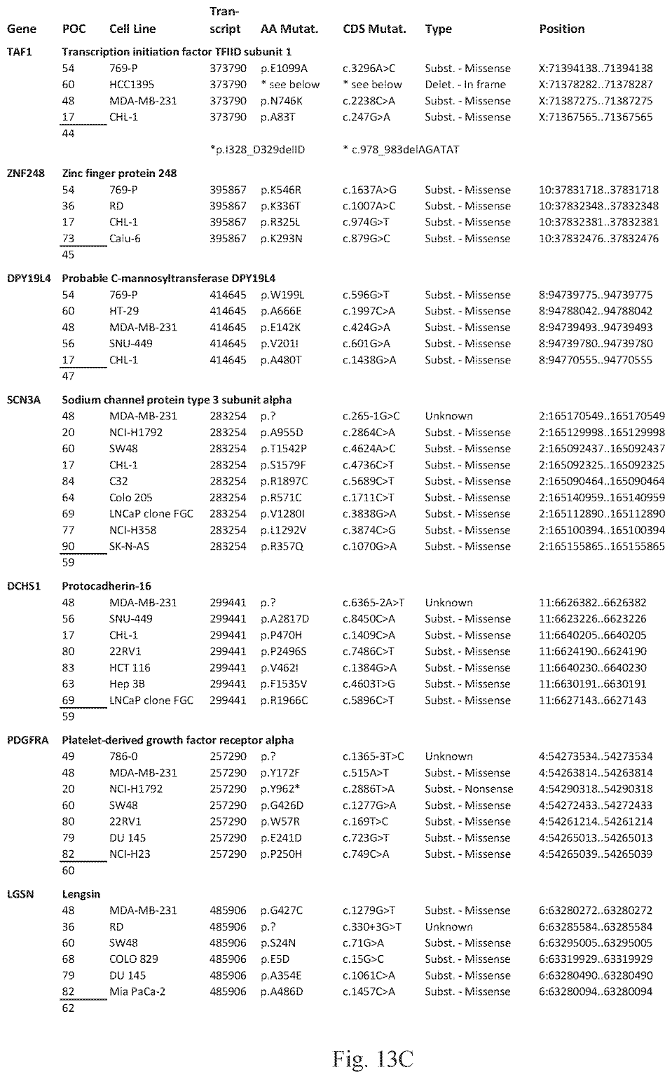

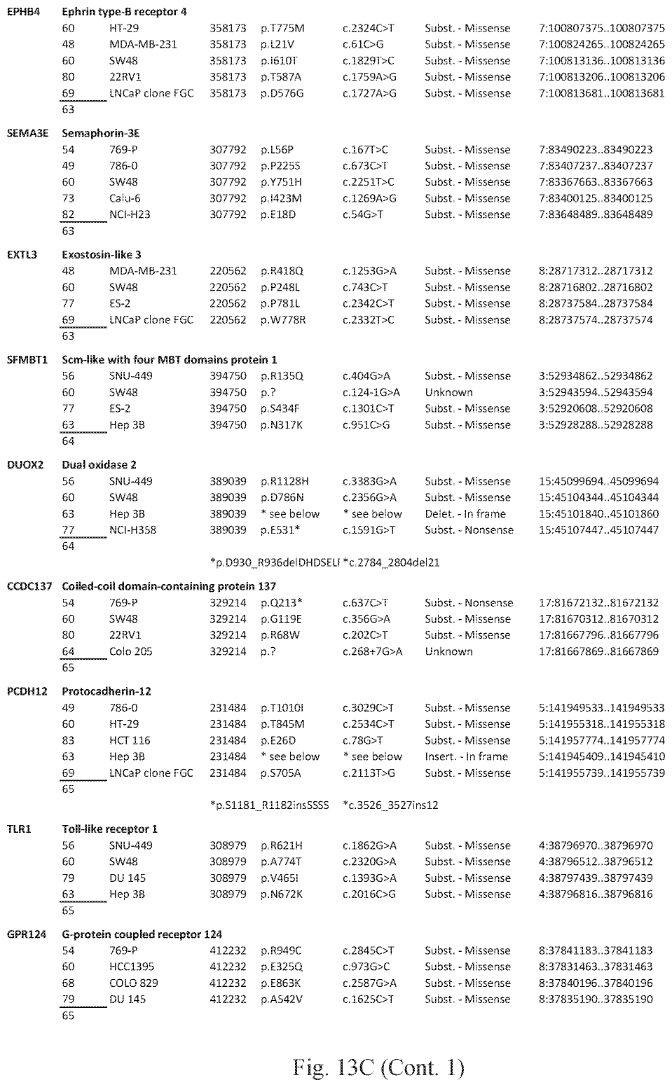

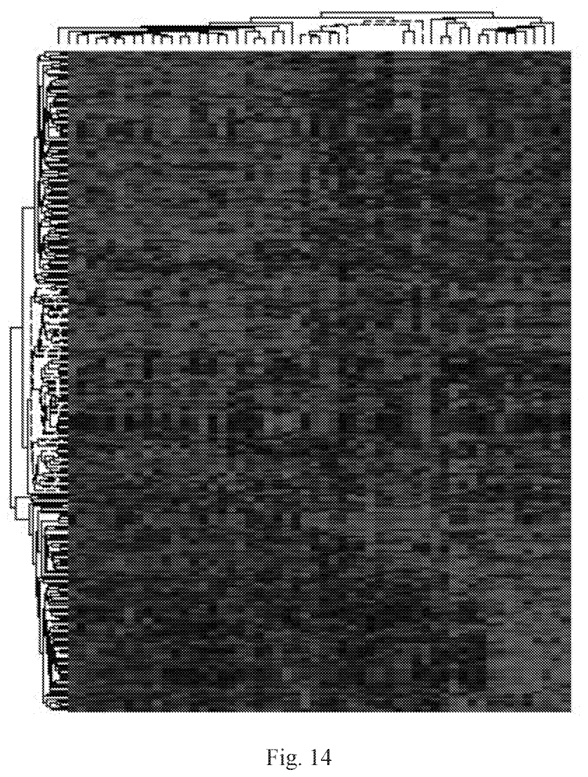

Except where otherwise indicated, the term “mutation” excludes silent mutations, in other words mutations that do not affect at least one of (a) the amino acid sequence of the protein encoded by the transcript; and (b) the splicing of the encoded transcript. The term includes nonsense mutations (mutations that introduce a premature stop codon), substitutions, deletions, insertions, inversions, and frameshift mutations, as well as mutations that affect splicing, for example mutations near splice sites. FIGS. 13B-C set forth non-limiting examples of mutations that are known to be present in tumor cells, which are provided merely for exemplification purposes. In more specific embodiments, the described mutation is a loss-of-function mutation. In certain embodiments, the mutation is a somatic mutation. In other embodiments, the mutation is a germline mutation.

Those skilled in the art will appreciate, in light of the present disclosure, that a variety of means are available to detect the presence of a mutation in a target gene or nucleotide sequence. Means for identifying mutations in the described genes or transcripts thereof (mRNA) are well known to one of skill in the art and include in particular and not by way of limitation, sequencing, selective hybridization and/or selective amplification. At the nucleic level, detection may be carried out on a sample of genomic DNA, mRNA or cDNA.

In various embodiments, sequencing may be complete or partial. In some embodiments, the means may include solely the sequencing of the region(s) comprising the residue(s) at which ASC treatment informative mutation(s) are located.

Non-limiting examples of such means include various DNA sequencing technologies and RNA sequencing technologies known in the art. DNA sequencing technologies include but are not limited to methods of sequencing genomic DNA, or a fraction thereof, present in a target cell or a lysate derived therefrom. RNA sequencing technologies include but are not limited to methods of sequencing RNA transcripts present in a target cell or a lysate derived therefrom. In some embodiments, high-throughput sequencing is utilized. This term is intended to encompass any technology capable of providing sequence information on multiple genes via a single test. Non-limiting examples of high-throughput sequencing technologies include Illumina (Solexa) sequencing technology, available commercially as MiSeqDx (Illumina, San Diego, Calif.); Roche 454 sequencing technology, available commercially as GS Junior and GS FLX+(454 Life Sciences, Branford, Conn.); and Ion torrent (Ion PGM™) sequencing, Ion Proton™ sequencing, and Supported Oligo Ligation Detection (SOLiD) sequencing technology, all available commercially from Thermo Fisher Scientific. Those skilled in the art will appreciate in light of the present disclosure how to apply such technologies to characterization of tumor cells. Descriptions of suitable systems, provided solely for exemplification purposes, are found in Hyman D M et al, Vijai J et al, and the references cited therein.

In other embodiments, selective hybridization is understood to mean that the genomic DNA, RNA or cDNA is placed in the presence of a probe specific for the mutant sequence(s) and optionally a probe specific for the target gene not harboring said mutation or the wild-type sequence. The probes may be, in various embodiments, in suspension or immobilized on a substrate. In some embodiments, the probes are labeled for easier detection. In more specific embodiments, the probes are single-stranded nucleic acid molecules of 8-1000 nucleotides, in other embodiments 10-800 or 15-50 nucleotides.

In some embodiments, the nucleic acid may be amplified before detection of the mutation. For instance, a primer pair specific of the regions flanking the region to be sequenced will be constructed. Typically, the primers are single-stranded nucleic acid molecules of 5-60 nucleotides, preferably 8-25 nucleotides. In some embodiments, the primers are perfectly complementary to the target sequence, which may be, in various embodiments, the wild-type sequence are a particular mutated sequence. However, some mismatches may be tolerated.

Once the target gene or the exon containing the mutation, or else one of its transcripts, has been amplified, the amplicon is used for detecting the presence of the mutation by sequencing or specific hybridization or by any other suitable method known to one of skill in the art. The mutation may also be detected by melting curve analysis (see WO2007/035806 for example).

In other embodiments, the presence of the mutation is detected by selective amplification of the mutant. For instance, a primer pair is prepared, one of the primers specifically hybridizing with the sequence carrying the mutation to be detected. Said primer will be able to initiate amplification or to hybridize with its target only if the sequence carries the mutated nucleotide. As a result, the presence of an amplicon would indicate that the target gene harbors the tested mutation, whereas the absence of said amplicon would indicate that the gene does not harbor this mutation.

It shall be understood that these methods may be readily adapted by one of skill in the art to detect simultaneously or in parallel several mutations of the target sequence.

Tumor Types

In certain embodiments, the described tumor (which is being tested for susceptibility to treatment with ASC or CM derived therefrom) is a cancer or neoplasm selected from: acute lymphoblastic leukemia, adrenocortical carcinoma, AIDS-related lymphoma, anal cancer, appendix cancer, astrocytoma (childhood cerebellar or cerebral), basal cell carcinoma, bile duct cancer, bladder cancer, bone cancer, brainstem glioma, brain tumor (cerebellar astrocytoma, cerebral astrocytoma/malignant glioma, ependymoma, medulloblastoma, supratentorial primitive neuroectodermal tumor, visual pathway and hypothalamic gliomas), breast cancer, bronchial adenoma, carcinoid tumor of the lung, gastric carcinoid, other carcinoid tumors (e.g. childhood), Burkitt lymphoma, carcinoma of unknown primary, central nervous system lymphoma (e.g. primary), cerebellar astrocytoma, malignant glioma (e.g. cerebral astrocytoma), cervical cancer, chronic lymphocytic leukemia, chronic myelogenous leukemia, colon cancer, cutaneous T-cell lymphoma, desmoplastic small round cell tumor, endometrial cancer, ependymoma, esophageal cancer, Ewing's sarcoma, extracranial germ cell tumor (e.g. childhood), extragonadal germ cell tumor, extrahepatic bile duct cancer, eye cancer (e.g. intraocular melanoma, retinoblastoma), gallbladder cancer, gastric (stomach) cancer, gastrointestinal stromal tumor, germ cell tumor (e.g. childhood extracranial), gestational trophoblastic tumor, hairy cell leukemia, head and neck cancer, hepatocellular (liver) cancer, Hodgkin lymphoma, other lymphomas (AIDS-related, non-Hodgkin, primary central nervous system), hypopharyngeal cancer, intraocular melanoma, islet cell carcinoma, Kaposi sarcoma, laryngeal cancer, leukemias (e.g. acute lymphoblastic, chronic lymphocytic, chronic myelogenous, hairy cell), lip and oral cavity cancer, primary liver cancer, small cell lung cancers, non-small cell lung cancer, macroglobulinemia (Waldenstrom), malignant fibrous histiocytoma of bone, medulloblastoma (e.g. childhood), intraocular melanoma, other melanomas, Merkel cell carcinoma, mesotheliomas (e.g. adult malignant, childhood), metastatic squamous neck cancer with occult primary, mouth cancer, multiple endocrine neoplasia syndrome (childhood), plasma cell neoplasms (e.g. multiple myeloma), mycosis fungoides, myelogenous leukemia (e.g. chronic), nasal cavity and paranasal sinus cancer, nasopharyngeal carcinoma, neuroblastoma, oral cancer, oropharyngeal cancer, osteosarcoma, ovarian cancer, ovarian epithelial cancer (e.g. surface epithelial-stromal tumor), ovarian germ cell tumor, ovarian low malignant potential tumor, islet cell pancreatic cancer, other pancreatic cancers, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, soft tissue sarcoma, uterine sarcoma, Sezary syndrome, melanoma, skin carcinoma (e.g. Merkel cell), other skin cancers, small intestine cancer, squamous cell carcinoma, supratentorial primitive neuroectodermal tumor (e.g. childhood), testicular cancer, throat cancer, thymoma (e.g. childhood), thymic carcinoma, thyroid cancer (childhood or adult), urethral cancer, endometrial uterine cancer, vaginal cancer, vulvar cancer, Waldenstrom macroglobulinemia, and Wilms tumor. In certain embodiments, the tumor is sensitive to TRAIL (also known as Tumor necrosis factor ligand superfamily member 10 or Apo-2L; Uniprot accession no. P50591. Uniprot was accessed on Dec. 29, 2015).

Those skilled in the art will appreciate, in light of the present disclosure, that the TRAIL-sensitivity of a cell or cell line can be readily determined. Exemplary protocols for doing so are described in James M A et al and the references cited therein. Exemplary protocols for confirming that tumor growth inhibition or death induction is TRAIL-mediated are described in the product literature for Anti-TRAIL antibody [75411.11] (ab 10516, Abcam), in Roux et al, and the references cited therein.

In other embodiments, the cancer or neoplasm that is tested for ASC sensitivity is selected from prostate carcinoma, urothelial bladder carcinoma, renal cell adenocarcinoma, gastric adenocarcinoma, pancreatic adenocarcinoma, breast ductal carcinoma, hepatocellular carcinoma, squamous cell carcinoma, thyroid anaplastic carcinoma, lung anaplastic carcinoma, melanoma, colorectal adenocarcinoma, glioblastoma, prostate carcinoma, ovarian clear cell carcinoma, uterine sarcoma, lung adenocarcinoma, bronchoalveolar carcinoma, large cell lung carcinoma, rhabdomyosarcoma, neuroblastoma, astrocytoma, and rectum adenocarcinoma. In certain embodiments, the tumor is TRAIL-sensitive.

In certain embodiments, the tumor is a breast tumor, which is in more specific embodiments a carcinoma, or in other embodiments an adenocarcinoma. In certain embodiments, the breast cancer has a mesenchymal phenotype. Those skilled in the art will appreciate that breast cancer cells with a mesenchymal phenotype have high expression levels of Vimentin (Uniprot accession no. P08670); and Caveolin-1 (Uniprot accession no. Q03135) and Caveolin-2 (Uniprot accession nos. P51636 and Q712N7), and low levels of E-cadherin (Uniprot accession no. P12830). Alternatively or in addition, the breast tumor is TRAIL-sensitive and/or is a triple-negative (TN) tumor. Those skilled in the art will appreciate that TN breast cancer cells lack receptors for estrogen (ER; Uniprot accession no. P03372) and progesterone (PR; Uniprot accession no. P06401), and do not have an amplification in human epidermal growth factor receptor 2 (HER2; Uniprot accession no. P04626) gene copy number or expression. The presence of these receptors can be readily ascertained, for example by fluorescence-activated cell sorting. The Uniprot entries mentioned in this paragraph were accessed on Dec. 29, 2015 or Jan. 3, 2016.

In other embodiments, the cancer or neoplasm that is treated, or in other embodiments prevented, by the described compositions is selected from metaplasias, dysplasias, neoplasias, and leukoplakias. In other embodiments, the cancer or neoplasm is selected from cancers of the breast, skin, prostate, colon, bladder, cervix, uterus, stomach, lung, esophagus, larynx, oral cavity. In still other embodiments, the cancer or neoplasm is a solid tumor, which is, in certain embodiments, selected from fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. In still other embodiments, the neoplasm is a papilloma of the mucous membranes. In certain embodiments, the tumor is TRAIL-sensitive.

In various embodiments, the cancer is non-Hodgkin lymphoma, colorectal cancer, malignant melanoma, thyroid carcinoma, non-small cell lung carcinoma, or lung adenocarcinoma. In yet other embodiments, the cancer or neoplasm is selected from non-Hodgkin lymphoma, colorectal cancer, malignant melanoma, thyroid carcinoma, and non-small cell lung carcinoma (e.g. lung adenocarcinoma). In certain embodiments, the tumor is TRAIL-sensitive.

In various other embodiments, the cancer or neoplasm is renal cell carcinoma, melanoma, breast carcinoma, hepatocellular carcinoma, colorectal adenocarcinoma, breast adenocarcinoma, lung adenocarcinoma, large cell lung carcinoma, or rhabdomyosarcoma. In various other embodiments, the cancer or neoplasm is selected from renal cell carcinoma, melanoma, breast carcinoma, hepatocellular carcinoma, colorectal adenocarcinoma, breast adenocarcinoma, lung adenocarcinoma, large cell lung carcinoma, and rhabdomyosarcoma. In more specific embodiments, the cancer or neoplasm is selected from renal cell carcinoma, hepatocellular carcinoma, and lung adenocarcinoma. In certain embodiments, the tumor is TRAIL-sensitive.

In the case of a solid tumor, the described ASC or a pharmaceutical composition comprising same are in some embodiments administered intra-tumorally; or in other embodiments, administered to the region of the body where the tumor is located; or in other embodiments, administered to the bed of an excised tumor to prevent recurrence of the neoplasm. In other embodiments, the ASC or composition is administered intramuscularly, subcutaneously, or systemically. In this regard, “intramuscular” administration refers to administration into the muscle tissue of a subject; “subcutaneous” administration refers to administration just below the skin; “intravenous” administration refers to administration into a vein of a subject; and “intratumoral” administration refers to administration within a tumor.

In still another embodiment is provided an article of manufacture, comprising (a) a packaging material, wherein the packaging material comprises a label describing a use testing a cancer, a tumor, or a neoplasm for susceptibility of treatment with ASC or CM derived therefrom.

Methods for determining the effect of cells (e.g. ASC) and solutions (e.g. CM) on the viability and replication of cancer cells are well known in the art. In some embodiments, 3D plates are utilized to house the target cancer cells, to encourage formation of cell cultures. An exemplary type of suitable plates is Elplasia™ plates, which are commercially available from Kuraray Co., Ltd. (Tokyo, JP). Use of such plates is described inter alia in Kobayashi K et al, Nakamura et al, and the references cited therein. Inhibition of replication and/or reduced tumor cell survival is evidence of therapeutic efficacy. Kits for determining the effects of cells and solutions on the viability and replication of cancer cells are commercially available from vendors such as Bioensis Preclinical Services. (Bellevue, Wash.). Methods for generating spheroids of cancer cells are well known in the art, and are described, for example, in Perche F et al, 2012, Friedrich J et al, 2009, Phung Y T et al 2011, KorffT et al 1998, Ivascu A et al 2006, and the references cited therein. In a non-limiting protocol, 10,000 cells are added into each well of polyHEMA-coated 96-well plates. The plates are briefly spun for 5 minutes at 800 rpm and then placed in a 37° C. humidified incubator with 5% CO² until spheroids form. Optionally, the basement membrane extract Matrigel™ may be added to the wells, in some embodiments as described in Ivascu A et al 2006. In another non-limiting protocol, microspheroids with an average of 250 cells each can be generated using nonadhesive hydrogels cast by micromolds. 3% agarose gels (Ultrapure agarose; Invitrogen, Carlsbad, Calif.) are cast by using micromolds, which produces recesses on the gel surface. The gels are then equilibrated overnight with complete culture medium. Trypsinized cells are resuspended to the appropriate cell density and then pipetted onto the gels. Over 24 hours (H1299) or 48 hours (A549), cells within the recesses form aggregates and are recovered from the gels by centrifugation. Other efficacy testing methods described herein are also suitable. Additionally, anti-cancer activity of ASC can be tested by in vivo models, using methods known in the art. Non-limiting examples of methods are described herein.

Additionally, animal tumor models are well known in the art, and include, inter alia, ectopic xenograft models, orthotopic xenograft models, genetically engineered tumor models, and carcinogen-induced tumor models. Such models are described inter alia in Ruggeri B A et al, Walker J D et al, Rocha N S et al, and the references cited therein. Methods for determining efficacy of anti-cancer treatment on human subjects are also well known in the art, and include tumor imaging, measurement of tumor marker proteins, and assessment of patient wellness, for example as described in Oh W K (Urol Oncol. 2003), Ramsey et al, and the references cited therein.

Methods for Preparing ASC

ASC can be propagated, in some embodiments, by using two-dimensional (“2D”) culturing conditions, three-dimensional (“3D”) culturing conditions, or a combination thereof. Conditions for propagating ASC in 2D and 3D culture are further described hereinbelow and in the Examples section which follows. These steps may be freely combined with any of the other described embodiments for culturing methods, characteristics of the cells, or therapeutic parameters, each of which is considered a separate embodiment.

As mentioned, in some embodiments, the cells have been propagated under 2D culturing conditions. The terms “2D culture” and “2D culturing conditions” refer to a culture in which the cells are exposed to conditions that are compatible with cell growth and allow the cells to grow in a monolayer, which is referred to as a “two-dimensional (2D) culture apparatus”. Such apparatuses will typically have flat growth surfaces, in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

In other embodiments, the cells have been propagated under 3D culturing conditions. The terms “3D culture” and “3D culturing conditions” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface, in some embodiments comprising an adherent material. Certain, non-limiting embodiments of 3D culturing conditions suitable for expansion of ASC are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a fibrous matrix, e.g. a woven fibrous matrix, a non-woven fibrous matrix, or either. In still other embodiments, the material exhibits a chemical structure that enables cell adhesion, for example charged surface-exposed moieties. Non-limiting examples of adherent materials which may be used in accordance with this aspect include polyesters, polypropylenes, polyalkylenes, poly fluoro-chloro-ethylenes, polyvinyl chlorides, polystyrenes, polysulfones, poly-L-lactic acids, cellulose acetate, glass fibers, ceramic particles, and inert metal fiber; or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and inert metal fibers. In more specific embodiments, the synthetic adherent material is selected from polyesters, polypropylenes, polyalkylenes, polyfluorochloroethylenes, polyvinyl chlorides, polystyrenes, polysulfones, cellulose acetates, and poly-L-lactic acids.

Alternatively or in addition, the described ASC have been incubated in a 2D adherent-cell culture apparatus, prior to the step of 3D culturing. In some embodiments, cells (following extraction from, in some embodiments, placenta, adipose tissue, etc.) are then subjected to prior step of incubation in a 2D adherent-cell culture apparatus, followed by the described 3D culturing steps. This step may be freely combined with any of the other described embodiments for culturing methods, characteristics of the cells, or therapeutic parameters, each of which is considered a separate embodiment.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

According to other embodiments, the described 3D culturing is performed for at least 4 doublings, at least 5 doublings, at least 6 doublings, at least 7 doublings, at least 8 doublings, at least 9 doublings, or at least 10 doublings. In certain embodiments, cells are passaged when the culture reaches about 70-90% confluence, typically after 3-5 days (e.g., 1-3 doublings).

In certain embodiments, 3D culturing is performed in a 3D bioreactor. In some embodiments, the 3D bioreactor comprises a container for holding medium and a 3D attachment (carrier) substrate disposed therein; and a control apparatus, for controlling pH, temperature, and oxygen levels, and optionally other parameters. Alternatively or in addition, the bioreactor contains ports for the inflow and outflow of fresh medium and gases.

Examples of bioreactors include, but are not limited to, a continuous stirred tank bioreactor, a CelliGen Plus® bioreactor system (New Brunswick Scientific [NBS]), and a BIOFLO 310 bioreactor system (NBS).

As provided herein, a 3D bioreactor is capable, in certain embodiments, of 3D expansion of ASC under controlled conditions (e.g. pH, temperature and oxygen levels) and with growth medium perfusion, which in some embodiments is constant perfusion and in other embodiments is adjusted in order to maintain target levels of glucose or other components. Non-limiting embodiments of target glucose concentrations are between 400-700 mg/liter, between 450-650 mg/liter, between 475-625 mg/liter, between 500-600 mg/liter, or between 525-575 mg/liter. Alternatively or in addition, the cell cultures can be directly monitored for concentrations of lactate, glutamine, glutamate and ammonium. The glucose consumption rate and the lactate formation rate of the adherent cells enable, in some embodiments, estimation of the cellular growth rate and determination of the optimal harvest time.

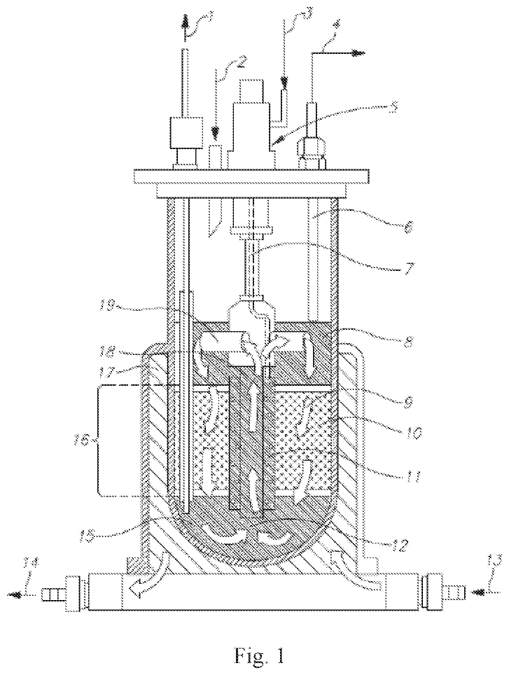

Another exemplary bioreactor, the Celligen 310 Bioreactor, is depicted in FIG. 1. A fibrous-bed basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

Alternatively or in addition, the adherent material is fibrous, which may be, in more specific embodiments, a woven fibrous matrix, a non-woven fibrous matrix, or either. In still other embodiments, the material exhibits a chemical structure such as charged surface groups, which allows cell adhesion, e.g. polyesters, polypropylenes, polyalkylenes, polyfluorochloroethylenes, polyvinyl chlorides, polystyrenes, polysulfones, cellulose acetates, and poly-L-lactic acids. In more particular embodiments, the material may be selected from a polyester and a polypropylene.

Alternatively or in addition, the carriers comprise a fibrous material, optionally an adherent, fibrous material, which may be, in more specific embodiments, a woven fibrous matrix, a non-woven fibrous matrix, or either. Non-limiting examples of fibrous carriers are New Brunswick Scientific Fibracel® carriers, available commercially from of Eppendorf AG, Germany, and made of polyester and polypropylene; and BioNOC II carriers, available commercially from CESCO BioProducts (Atlanta, Ga.) and made of PET (polyethylene terephthalate). In certain embodiments, the referred-to fibrous matrix comprises a polyester, a polypropylene, a polyalkylene, a polyfluorochloroethylene, a polyvinyl chloride, a polystyrene, or a polysulfone. In more particular embodiments, the fibrous matrix is selected from a polyester and a polypropylene.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus similar to the Celligen™ Plug Flow bioreactor which is, in certain embodiments, packed with Fibra-cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the agitation speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed and optionally washed with isotonic buffer, and cells are processed or removed from the carriers by agitation and/or enzymatic digestion.

In certain embodiments, the 3D growth apparatus (in some embodiments the aforementioned bioreactor) contains a fibrous bed. In more specific embodiments, the fibrous bed may contain polyester, polypropylene, polyalkylene, poly fluoro-chloro-ethylene, polyvinyl chloride, polystyrene, polysulfone, or a polyamide (e.g. an aliphatic polyamide). In other embodiments, glass fibers or metal fibers (e.g. inert metal fibers) may be present; or a cellulose fiber (a non-limiting example of which is rayon) may be present.

In other embodiments, incubation of ASC may comprise microcarriers, which may, in certain embodiments, be inside a bioreactor. Microcarriers are well known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.) Superbeads (commercially available from Flow Labs, Inc.), and as DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

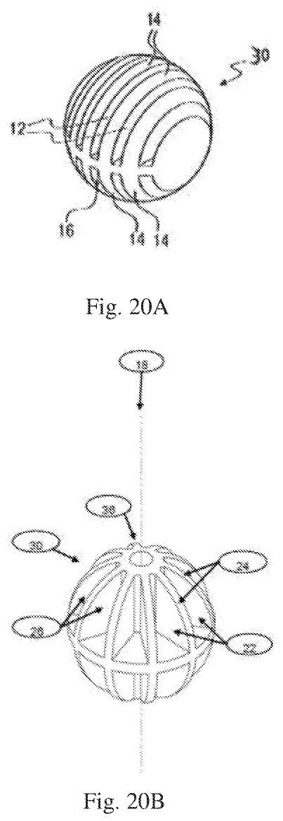

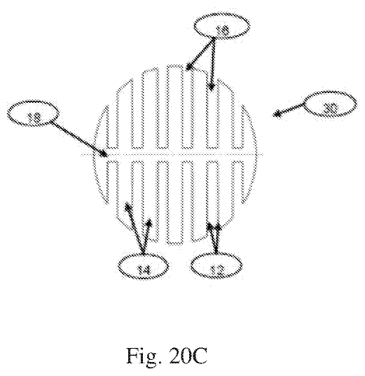

In some embodiments, with reference to FIGS. 20A-B, and as described in WO/2014/037862, published on Mar. 13, 2014, which is incorporated herein by reference in its entirety, grooved carriers 30 are used for proliferation and/or incubation of ASC. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate.

Carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 20C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In more specific embodiments, the central carrier axis 18 is a plane that bisects the sphere, and openings 16 extend from the surface of the carrier to the proximal surface of the plane. In some embodiments, carriers 30 are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In the embodiment shown in FIG. 20A, ribs 14 are substantially flat and extend parallel to one another. In other embodiments, the ribs are in other configurations. For example, FIG. 20B illustrates carrier 30 having multiple two-dimensional surfaces 22 formed by ribs 24 in a different configuration. In particular, ribs 24 are shaped to form openings 26 that are spaced around the circumference of carrier 30, whereby openings 26 can be generally wedge shaped. Ribs 24 can extend generally radially from a central carrier axis 18 of carrier 30 to a peripheral surface of carrier 30. Carrier 30 can also include one or more lateral planes extending from the central carrier axis 18 of carrier 30 and extending generally perpendicular to ribs 24, as depicted in FIG. 20C, which is a cross-sectional view of certain embodiments of the carrier 30 of FIG. 20A. Further, carrier 30 includes an opening 36 extending through the carrier's center and forming additional surfaces 32, which can support monolayer growth of eukaryotic cells.

In still other embodiments, the material forming the multiple 2D surfaces comprises at least one polymer. In more specific embodiments, the polymer is selected from a polyamide, a polycarbonate, a polysulfone, a polyester, a polyacetal, and polyvinyl chloride.

In various embodiments, the described grooved carriers are coated with one or more coatings. Suitable coatings may, in some embodiments, be selected to control cell attachment or parameters of cell biology. Suitable coatings may include, for example, peptides, proteins, carbohydrates, nucleic acid, lipids, polysaccharides, glycosaminoglycans, proteoglycans, hormones, extracellular matrix molecules, cell adhesion molecules, natural polymers, enzymes, antibodies, antigens, polynucleotides, growth factors, synthetic polymers, polylysine, drugs and/or other molecules or combinations or fragments of these.

In some embodiments, incubation in the described grooved carriers takes place inside a bioreactor.

In certain embodiments, the ASC have been incubated in a 2D adherent-cell culture apparatus, for example tissue culture plates, prior to the incubation in the described grooved carriers.

Alternatively or in addition, the ASC are incubated in a 3D adherent-cell culture apparatus, following the described incubation in grooved carriers.

In certain embodiments, the method of expanding the ASC further comprises the subsequent step (following the described 3D incubation, which may be, in various embodiments, with or without added cytokines) of harvesting the ASC by removing the ASC from the 3D culture apparatus. In more particular embodiments, cells may be removed from a 3D matrix while the matrix remains within the bioreactor. In certain embodiments, at least about 10%, at least 12%, at least 14%, at least 16%, at least 18%, at least 20%, at least 22%, at least 24%, at least 26%, at least 28%, or at least 30% of the cells are in the S and G2/M phases (collectively), at the time of harvest from the bioreactor. Cell cycle phases can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In still other embodiments, the harvest utilizes vibration, for example as described in PCT International Application Publ. No. WO 2012/140519, which is incorporated herein by reference. This step may be freely combined with any of the other described embodiments for culturing methods, characteristics of the cells, or therapeutic parameters, each of which is considered a separate embodiment. In certain embodiments, during harvesting, the cells are vibrated at 0.7-6 Hertz, or in other embodiments 1-3 Hertz, during, or in other embodiments during and after, treatment with protease plus a calcium chelator, non-limiting examples of which are trypsin, or another enzyme with similar activity, with EDTA. Enzymes with similar activity to trypsin are well known in the art; a non-limiting example is a fungal trypsin-like protease, TrypLE™, which is available commercially from Life Technologies. In more specific embodiments, the total duration of vibration during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to vibration at 0.7-6 Hertz, or in other embodiments 1-3 Hertz, during the wash step before the protease and calcium chelator are added.

In certain embodiments, the ASC used as an anti-cancer agent have been previously co-incubated with cancer cells, or, in other embodiments, with one or more cancer cell lines incubated in conditioned medium (“CM”) derived from cancer cells or cancer cell lines, or have been incubated in medium containing a fraction of a CM derived from cancer cells or cancer cell lines. In other embodiments, the ASC used to produce CM for use as an anti-cancer agent have been co-incubated with cancer cells, or, in other embodiments, with one or more cancer cell lines. In some embodiments, the co-incubation is performed under conditions where the ASC and cancer cells or cell lines contact one another. Such conditions include seeding the ASC and cancer cells or cell lines in the same apparatus, in various embodiments either together, first seeding the ASC, or first seeding the cancer cells or cell lines. The co-incubation takes place, in some embodiments, in a tissue culture apparatus, or in other embodiments, in a bioreactor, which may in some embodiments comprise a 3D growth substrate.

In other embodiments, the conditions are such that the ASC and cancer cells or cell lines do not contact one another, but medium and soluble components thereof are exchanged between the two cell populations. Those skilled in the art will appreciate that various means are available to prevent contact between two cell populations while permitting exchange of medium, for example by separating the cell populations with a membrane that is permeable to fluids and factors dissolved therein, or a semi-permeable membrane that allows soluble factors smaller than a defined size to diffuse through it.

In other embodiments, the ASC used as an anti-cancer agent have been previously incubated in CM derived from cancer cells or cancer cell lines. In other embodiments, the ASC used to produce CM for use as an anti-cancer agent have been incubated in CM derived from cancer cells or cancer cell lines. In some embodiments of the first stage of the process, cancer cells are cultured, and the medium resulting from the incubation (the “cancer cell CM”) is isolated. In certain embodiments of the second stage, ASC are incubated with the medium generated in the first step, for example in a bioreactor, or in culture wells. The incubation of the ASC takes place, in some embodiments, in a tissue culture apparatus, or in other embodiments, in a bioreactor, which may in some embodiments comprise a 3D growth substrate. In still other embodiments, the ASC have been exposed to inflammatory cytokines, prior to their incubation in the cancer cell CM. In other embodiments, or one or more cytokines, vitamins, or biologically active proteins are added to the cancer cell CM. In still other embodiments, the incubation of the ASC is performed under non-standard conditions, for example hypoxia or altered pH or atmospheric pressure. In still other embodiments, the CM resulting from the incubation of the ASC (the “ASC CM”), or in other embodiments the ASC themselves, is used as an anti-cancer agent. In yet other embodiments, the ASC are placental ASC that are predominantly maternal cells, or are fetal cells, or are a mixture of fetal and maternal cells.

In other embodiments, the ASC used as an anti-cancer agent have been incubated in medium containing a fraction of a CM derived from cancer cells or cancer cell lines. In other embodiments, the ASC used to produce CM for use as an anti-cancer agent have been incubated in medium containing a fraction of a CM derived from cancer cells or cancer cell lines. In some embodiments, the process comprises a first stage, wherein cancer cells are cultured, and a fraction of the medium resulting from the incubation (the “cancer cell CM”) is isolated and added to a standard culture medium. In certain embodiments, the process comprises a second stage, in which ASC are incubated with the medium generated in the first stage, for example in a bioreactor, or in culture wells. The incubation takes place, in some embodiments, in a tissue culture apparatus, or in other embodiments, in a bioreactor, which may in some embodiments comprise a 3D growth substrate. In still other embodiments, the ASC have been exposed to inflammatory cytokines, prior to their incubation in the medium containing cancer cell factors. In other embodiments, or one or more cytokines, vitamins, or biologically active proteins are added to the medium containing cancer cell factors. In still other embodiments, the incubation of the ASC is performed under non-standard conditions, for example hypoxia or altered pH or atmospheric pressure. Lastly, the CM resulting from the incubation of the ASC (the “ASC CM”), or the ASC themselves, is used as an anti-cancer agent. In yet other embodiments, the ASC are placental ASC that are predominantly maternal cells, or are fetal cells, or are a mixture of fetal and maternal cells.

In certain embodiments, the conditions of the aforementioned incubation are such that the cancer cells or cell lines form spheroids, or in other embodiments form microspheroids, during the co-incubation.

In various embodiments, incubation of ASC with cancer cells or cancer cell lines, or with CM derived therefrom, is performed after 3D expansion of the ASC as described herein, in the absence of cancer cells, cancer cell lines, or CM derived therefrom. In other embodiments, the entire process of 3D expansion of the ASC is performed in the presence of cancer cells, cancer cell lines, or CM derived therefrom. In still other embodiments, expansion of the ASC in the absence of cancer cells, cancer cell lines, or CM derived therefrom occurs after co-incubation of the ASC with cancer cells, cancer cell lines, or CM derived therefrom. In certain embodiments, the 2D expansion of the ASC is performed before 3D incubation of ASC without and/or with cancer cells, cancer cell lines, or CM derived therefrom.

In certain embodiments, the ASC have been exposed to inflammatory cytokines, for example while in the bioreactor used to expand them, prior to performing an additional incubation with cancer cells or cancer cell lines. In other embodiments, the ASC have been exposed to inflammatory cytokines, following (i) growing the ASC in a bioreactor, (ii) optionally harvesting them from the bioreactor, and (iii) performing an additional incubation of the ASC with cancer cells or cancer cell lines. In various embodiments, the additional incubation is performed in culture plates, optionally under non-standard conditions, for example hypoxia or altered pH or atmospheric pressure.

In other embodiments, one or more cytokines, vitamins, or biologically active proteins are added to the medium used for the co-incubation.

In other embodiments, the co-incubation is performed under non-standard conditions, for example hypoxia or altered pH or atmospheric pressure.

In certain embodiments, the cell lines used in the co-incubation need not be the same type of cancer cell that is the therapeutic target of the obtained ASC or CM. In other embodiments, the cell lines used in the co-incubation are the same type of cancer cell that is the therapeutic target of the obtained ASC or CM

In various embodiments, the ASC used in each of the described co-incubation methods may utilize placental ASC that are predominantly maternal cells, or are predominantly fetal cells, or are a mixture of fetal cells and maternal cells. Each of these embodiments may be freely combined with the described embodiments of co-incubation of ASC with cancer cells or cancer cell lines.

Cells Subjected to Pro-Inflammatory Cytokines

In certain embodiments of the described methods and compositions, the composition of the medium is not varied during the course of the 3D culture used to prepare the ASC. In other words, no attempt is made to intentionally vary the medium composition by adding or removing factors or adding fresh medium with a different composition than the previous medium. Reference to varying the composition of the medium does not include variations in medium composition that automatically occur as a result of prolonged culturing, for example due to the absorption of nutrients and the secretion of metabolites by the cells therein, as will be appreciated by those skilled in the art.

In other embodiments, the 3D culturing method used to prepare the cells comprises the sub-steps of: (a) incubating ASC in a 3D culture apparatus in a first growth medium, wherein no inflammatory cytokines have been added to the first growth medium; and (b) subsequently incubating the ASC in a 3D culture apparatus in a second growth medium, wherein one or more pro-inflammatory cytokines have been added to the second growth medium. Those skilled in the art will appreciate, in light of the present disclosure, that the same 3D culture apparatus may be used for the incubations in the first and second growth medium by simply adding cytokines to the medium in the culture apparatus, or, in other embodiments, by removing the medium from the culture apparatus and replacing it with medium that contains cytokines. In other embodiments, a different 3D culture apparatus may be used for the incubation in the presence of cytokines, for example by moving (e.g. passaging) the cells to a different incubator, before adding the cytokine-containing medium. Those skilled in the art will appreciate, in light of the present disclosure, that the ASC to be used in the described methods may be extracted, in various embodiments, from the placenta, from adipose tissue, or from other sources, as described further herein.

Reference herein to one or more “pro-inflammatory” cytokines, or “inflammatory cytokines”, which are used interchangeably, implies the presence of at least one cytokine that mediates an inflammatory response in a mammalian host, for example a human host. A non-limiting list of cytokines are Interferon-gamma (IFN-gamma; UniProt identifier P01579), IL-22 (UniProt identifier Q9GZX6), Tumor Necrosis Factor-alpha (TNF-alpha; UniProt identifier P01375), IFN-alpha, IFN-beta (UniProt identifier P01574), IL-lalpha (UniProt identifier P01583), IL-1beta (UniProt identifier P01584), IL-17 (UniProt identifier Q5QEX9), IL-23 (UniProt identifier Q9NPF7), IL-17A (UniProt identifier Q16552), IL-17F (UniProt identifier Q96PD4), IL-21 (UniProt identifier Q9HBE4), IL-13 (UniProt identifier P35225), IL-5 (UniProt identifier P05113), IL-4 (UniProt identifier P05112), IL-33 (UniProt identifier 095760), IL-1RL1 (UniProt identifier Q01638), TNF-Beta (UniProt identifier P01374), IL-11 (UniProt identifier P20809), IL-9 (UniProt identifier P15248), IL-2 (UniProt identifier P60568), IL-21 (UniProt identifier Q9HBE4), Tumor Necrosis Factor-Like Ligand (TL1A; a.k.a. TNF ligand superfamily member 15; UniProt identifier 095150), IL-12 (UniProt identifiers P29459 and P29460 for the alpha- and beta subunits, respectively), and IL-18 (UniProt identifier Q14116). Additional cytokines include (but are not limited to): Leukemia inhibitory factor (LIF; UniProt identifier P15018), oncostatin M (OSM; UniProt identifier P13725), ciliary neurotrophic factor (CNTF (UniProt identifier P26441), and IL-8 (UniProt identifier P10145). All Swissprot and UniProt entries in this paragraph were accessed on Jul. 24, 2014.

Except where indicated otherwise, reference to a cytokine or other protein is intended to include all isoforms of the protein. For example, IFN-alpha includes all the subtypes and isoforms thereof, such as but not limited to IFN-alpha 17, IFN-alpha 4, IFN-alpha 7, IFN-alpha 8, and IFN-alpha 110. Some representative UniProt identifiers for IFN-alpha are P01571, P05014, P01567, P32881, and P01566. Those skilled in the art will appreciate that, even in the case of human cells, the aforementioned cytokines need not be human cytokines, since many non-human (e.g. animal) cytokines are active on human cells. Similarly, the use of modified cytokines that have similar activity to the native forms falls within the scope of the described methods and compositions.

In certain embodiments, the cytokine present in the described medium, or in other embodiments at least one of the cytokines present, if more than one is present, is an inflammatory cytokine that affects innate immune responses. In further embodiments, the cytokine is one of, or in other embodiments more than one, of TNF-α, IL-1alpha, IL-12, IFN-α IFN-β, or IFN-γ.

In certain embodiments, one or more of the cytokines is TNF-alpha. In more specific embodiments, the TNF-alpha may be the only cytokine present, or, in other embodiments, may be present together with 1, 2, 3, 4, 5, 6, 1-2, 1-3, 1-4, 1-5, or 1-6, or more than 6 added inflammatory cytokines, which may be, in certain embodiments, one of the aforementioned cytokines. In more specific embodiments, TNF-alpha is present in an amount of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

In some embodiments, TNF-alpha is present in the medium together with IFN-gamma. These two cytokines may be the only added cytokines, or, in other embodiments, present with additional proinflammatory cytokines. In still other embodiments, IFN-gamma and TNF-alpha are each present in an amount independently selected from one of the aforementioned amounts or ranges. Each combination may be considered as a separate embodiment. In still other embodiments, the amounts of IFN-gamma and TNF-alpha are both within the range of 5-20 ng/ml; or are both within the range of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

As mentioned, in some embodiments, TNF-alpha is present together with one, or in other embodiments 2, 3, 4, 5, or more than 5, of the aforementioned cytokines. In still other embodiments, TNF-alpha and one, or in other embodiments more than one, of the additional cytokines is each present in an amount independently selected from one of the aforementioned amounts or ranges. Each combination may be considered as a separate embodiment. In still other embodiments, the amounts of TNF-alpha and the other cytokine(s) are both within the range of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

In certain embodiments, one or more of the cytokines is IFN-gamma. In more specific embodiments, the IFN-gamma may be the only cytokine present, or, in other embodiments, may be present together with 1, 2, 3, 4, 5, 6, 1-2, 1-3, 1-4, 1-5, or 1-6, or more than 6 added cytokines. In more specific embodiments, IFN-gamma is present in an amount of 1-100 ng/ml; 2-100 ng/ml; 3-100 ng/ml; 4-100 ng/ml; 5-100 ng/ml; 7-100 ng/ml; 10-100 ng/ml; 15-100 ng/ml; 20-100 ng/ml; 30-100 ng/ml; 40-100 ng/ml; 50-100 ng/ml; 1-50 ng/ml; 2-50 ng/ml; 3-50 ng/ml; 4-50 ng/ml; 5-50 ng/ml; 7-50 ng/ml; 10-50 ng/ml; 20-50 ng/ml; 1-30 ng/ml; 2-30 ng/ml; 3-30 ng/ml; 4-30 ng/ml; 5-30 ng/ml; 6-30 ng/ml; 8-30 ng/ml; 10-30 ng/ml; 15-30 ng/ml; 1-20 ng/ml; 2-20 ng/ml; 3-20 ng/ml; 4-20 ng/ml; 5-20 ng/ml; 6-20 ng/ml; 8-20 ng/ml; 10-20 ng/ml; 5-15 ng/ml; 6-15 ng/ml; 6-14 ng/ml; 7-13 ng/ml; 8-12 ng/ml; 9-11 ng/ml; 9.5-10.5 ng/ml; 1-10 ng/ml; 1-8 ng/ml; 1-6 ng/ml; 1-5 ng/ml; 1-4 ng/ml; 2-10 ng/ml; 2-8 ng/ml; 2-6 ng/ml; 2-5 ng/ml; or 2-4 ng/ml.

In certain embodiments, the ASC, prior to their ex vivo exposure to cytokines, are placental-derived, adipose-derived, or bone marrow (BM)-derived ASC. Alternatively or in addition, the ASC are mesenchymal-like ASC, which exhibit a marker pattern similar to “classical” MSC, but do not differentiate into osteocytes, under conditions where “classical” MSC would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where MSC would differentiate into osteocytes or adipocytes, respectively. The MSC used for comparison in these assays are, in one embodiment, MSC that have been harvested from BM and cultured under 2D conditions. In other embodiments, the MSC used for comparison have been harvested from BM and cultured under 2D conditions, followed by 3D conditions. In more particular embodiments, the mesenchymal-like ASC are maternal cells, or in other embodiments are fetal cells, or in other embodiments are a mixture of fetal cells and maternal cells.

In certain embodiments, the ASC, following their ex vivo exposure to cytokines, exhibit a marker pattern similar to “classical” MSC, but do not differentiate into osteocytes, under conditions where “classical” MSC would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where MSC would differentiate into osteocytes or adipocytes, respectively. The MSC used for comparison in these assays are, in one embodiment, MSC that have been harvested from BM and cultured under 2D conditions. In other embodiments, the MSC used for comparison have been harvested from BM and cultured under 2D conditions, followed by 3D conditions. In more particular embodiments, the mesenchymal-like ASC are maternal cells, or in other embodiments are fetal cells, or in other embodiments are a mixture of fetal cells and maternal cells.

Optional Additional Preparation Steps

In certain embodiments, further steps of purification or enrichment for ASC may be performed as part of the cell preparation process. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that expression of various cell surface markers can be detected by staining, followed by fluorescence-activated cell sorting (FACS), and that data from one or more markers can be used individually or in combination in the sorting process.

Buffers

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, whether or not inflammatory cytokines are added, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), salts (e.g. B-glycerophosphate), steroids (e.g. dexamethasone) and hormones, e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

Those skilled in the art will appreciate that animal sera and other sources of growth factors are often included in growth media. In some cases, animal sera may contain inflammatory cytokines, which, in general, are not present in large amounts. Some preparations utilize a serum that is treated, for example, with charcoal, so as to remove most or all of the cytokines present. In any event, reference herein to “added cytokines”, “medium containing cytokines”, or the like, does not encompass the presence of cytokines present in animal sera that is customarily included in the medium.

It will also be appreciated that in certain embodiments, when the described ASC are intended for administration to a human subject, the cells and the culture medium (e.g., with the above described medium additives) are substantially xeno-free, i.e., devoid of any animal contaminants e.g., mycoplasma. For example, the culture medium can be supplemented with a serum-replacement, human serum and/or synthetic or recombinantly produced factors.

The various media described herein, i.e. (as applicable) the 2D growth medium, the first 3D growth medium, and/or the second 3D growth medium, may be independently selected from each of the described embodiments relating to medium composition. In certain embodiments, the only difference between the first and second 3D growth media is the presence of the added cytokines. In other embodiments, the first and second 3D growth media differ in other respects. In various embodiments, any medium suitable for growth of cells in a bioreactor may be used.

Tissue Sources and Cell Characteristics

In certain embodiments, the described ASC (e.g. prior to incubation with inflammatory cytokines, where appropriate) are mesenchymal stromal cells (MSC). These cells may, in some embodiments, be isolated from many adult tissues, such as placenta, bone marrow and adipose. In further embodiments, the cells are human MSC as defined by The Mesenchymal and Tissue Stem Cell Committee of the International Society for Cellular Therapy (Dominici et al, 2006), based on the following 3 criteria: 1. Plastic-adherence when maintained in standard culture conditions (α minimal essential medium plus 20% fetal bovine serum (FBS)). 2. Expression of the surface molecules CD105, CD73 and CD90, and lack of expression of CD45, CD34, CD14 or CD11b, CD79α or CD19 and HLA-DR. 3. Differentiation into osteoblasts, adipocytes and chondroblasts in vitro.

Alternatively or in addition, the described ASC are mesenchymal-like ASC cells, which exhibit a marker pattern similar to “classical” MSC, but do not differentiate into osteocytes, under conditions where “classical” MSC would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where MSC would differentiate into osteocytes or adipocytes, respectively. The MSC used for comparison in these assays are, in one embodiment, MSC that have been harvested from BM and cultured under 2D conditions. In other embodiments, the MSC used for comparison have been harvested from BM and cultured under 2D conditions, followed by 3D conditions. In more particular embodiments, the mesenchymal-like ASC are maternal cells, or in other embodiments are fetal cells, or in other embodiments are a mixture of fetal cells and maternal cells.

In various embodiments, ASC may be derived, for example, from placenta; adipose tissue; bone marrow; peripheral blood; umbilical cord blood; synovial fluid; synovial membranes; spleen; thymus; mucosa (for example nasal mucosa); limbal stroma; ligaments, for example the periodontal ligament; scalp; hair follicles, testicles; embryonic yolk sac; and amniotic fluid, all of which are known to include ASC. In certain embodiments, the source of the ASC is a non-fetal source, for example maternal cells from the placenta or somatic tissue from a pediatric or adult donor, for example adipose tissue, bone marrow, peripheral blood, umbilical cord blood, synovial fluid, synovial membranes, and ligaments such as the periodontal ligament. In some embodiments, the ASC are human ASC, while in other embodiments, they may be animal ASC. In particular embodiments, the ASC are derived from placental tissue or are derived from adipose tissue.

Placenta-Derived Stromal Cells

Except where indicated otherwise herein, the terms “placenta”, “placental tissue”, and the like refer to any portion of the placenta. Placenta-derived ASC may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (Falcon, Becton, Dickinson, San Jose, Calif.) with washing medium. In some embodiments, the tissue treatment includes use of a DNAse, a non-limiting example of which is Benzonase from Merck. In other embodiments, placental cells may be obtained from a full-term or pre-term placenta.

A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

Placental Cell Preparations Enriched for Fetal Cells or Maternal Cells

In other embodiments, the described ASC are a placental preparation containing both maternal and fetal cells. In certain embodiments, the preparation is enriched for maternal cells. Under many standard culture conditions, maternal cells tend to dominate 2D and 3D cultures after several passages. In other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are maternally-derived cells. Lack of expression of CD200, as measured by flow cytometry, using an isotype control to define negative expression, can be used as a marker of fetal cells.

Methods of preparing and characterizing maternal-derived and fetal-derived ASC are described in WO 2011/064669, which is incorporated herein by reference. In some embodiments, maternal and fetal placental ASC are identified based on genotype and/or karyotype (e.g., FISH) analysis. For example, ASC from a placenta of a male embryo can be separated into fetal and maternal cells based on karyotype analysis (i.e., XX cells are maternal while XY cells are fetal). In some embodiments, ASC derived from a fetal portion of the placenta (e.g., consisting of or comprising chorionic villi) express CD200. In other embodiments, not more than 3.5%, not more than 3%, not more than 2%, or not more than 1% of the adherent stromal cells from a maternal placental cell preparation express CD200 as measured by flow cytometry using an isotype control to define negative expression.

In other embodiments, the preparation is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In still other embodiments, the preparation is a placental cell population that is a mixture of fetal and maternal cells. In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

Adipose-Derived Stromal Cells

As used herein the phrase “adipose tissue” refers to a connective tissue which comprises fat cells (adipocytes). Adipose tissue-derived adherent stromal cells may be extracted, in various embodiments, by a variety of methods known to those skilled in the art, for example those described in U.S. Pat. No. 6,153,432, which is incorporated herein by reference. The adipose tissue may be derived, in other embodiments, from omental/visceral, mammary, gonadal, or other adipose tissue sites. In some embodiments, the adipose can be isolated by liposuction.

In other embodiments, ASC may be derived from adipose tissue by treating the tissue with a digestive enzyme (non-limiting examples of which are collagenase, trypsin, dispase, hyaluronidase or DNAse); and ethylenediaminetetraacetic acid (EDTA). The cells may be, in some embodiments, subjected to physical disruption, for example using a nylon or cheesecloth mesh filter. In other embodiments, the cells are subjected to differential centrifugation directly in media or over a Ficoll™, Percoll™, or other particulate gradient (see U.S. Pat. No. 7,078,230, which is incorporated herein by reference).

Stromal Cells from Other Sources

As mentioned, in some embodiments the source of the ASC is a non-fetal source, for example maternal cells from the placenta or somatic tissue from a pediatric or adult donor, for example adipose tissue, bone marrow, peripheral blood, umbilical cord blood, synovial fluid, synovial membranes, and ligaments such as the periodontal ligament. Those skilled in the art will appreciate in light of the present disclosure that ASC may be extracted from various body tissues, using standard techniques such as physical and/or enzymatic tissue disruption, and then may be subjected to the culturing methods described herein.

Identifying Characteristics

As mentioned, in some embodiments, the described ASC, prior to incubation with inflammatory cytokines, where relevant, do not differentiate into osteocytes, under conditions where “classical” mesenchymal stem cells would differentiate into osteocytes. In some embodiments, the conditions are incubation with a solution containing 0.1 micromolar (mcM) dexamethasone, 0.2 mM ascorbic acid, and 10 mM glycerol-2-phosphate, in plates coated with vitronectin and collagen, for 17 days. In still other embodiments, the conditions are incubation with a solution containing 10 mcM dexamethasone, 0.2 mM ascorbic acid, 10 mM glycerol-2-phosphate, and 10 nM Vitamin D, in plates coated with vitronectin and collagen, for 26 days. The aforementioned solutions will typically contain cell culture medium such as DMEM+10% serum or the like, as will be appreciated by those skilled in the art.

In other embodiments, the described ASC, prior to incubation with inflammatory cytokines, where relevant, do not differentiate into adipocytes, under conditions where mesenchymal stem cells would differentiate into adipocytes. In some embodiments, as provided herein, the conditions are incubation of adipogenesis induction medium, namely a solution containing 1 mcM dexamethasone, 0.5 mM 3-Isobutyl-1-methylxanthine (IBMX), 10 mcg/ml insulin, and 100 mcM indomethacin, added on days 1, 3, 5, 9, 11, 13, 17, 19, and 21, while the medium is replaced with adipogenesis maintenance medium, namely a solution containing 10 mcg/ml insulin, on days 7 and 15, for a total of 25 days. In still other embodiments, as provided herein, a modified adipogenesis induction medium, containing 1 mcM dexamethasone, 0.5 mM IBMX, 10 mcg/ml insulin, and 200 mcM indomethacin, is used, and the incubation is for a total of 26 days. The aforementioned solutions will typically contain cell culture medium such as DMEM+10% serum or the like, as will be appreciated by those skilled in the art.

In other embodiments, the described ASC, prior to incubation with inflammatory cytokines, where relevant, exhibit a spindle shape when cultured under 2D conditions.

Alternatively or additionally, the ASC, prior to incubation with inflammatory cytokines, where relevant, may express a marker or a collection of markers (e.g. surface marker) characteristic of MSC or mesenchymal-like stromal cells. Examples of surface markers include but are not limited to CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). Examples of markers expected to be absent from stromal cells are CD3 (UniProtKB Accession Nos. P09693 [gamma chain] P04234 [delta chain], P07766 [epsilon chain], and P20963 [zeta chain]), CD4 (UniProtKB Accession No. P01730), CD34 (UniProtKB Accession No. P28906), CD45 (UniProtKB Accession No. P08575), CD80 (UniProtKB Accession No. P33681), CD19 (UniProtKB Accession No. P15391), CD5 (UniProtKB Accession No. P06127), CD20 (UniProtKB Accession No. P11836), CDIlB (UniProtKB Accession No. P11215), CD14 (UniProtKB Accession No. P08571), CD79-alpha (UniProtKB Accession No. B5QTD1), and HLA-DR (UniProtKB Accession Nos. P04233 [gamma chain], P01903 [alpha chain], and P01911 [beta chain]). All UniProtKB entries were accessed on Jul. 7, 2014, except where indicated otherwise. Those skilled in the art will appreciate that the presence of complex antigens such as CD3 and HLA-DR may be detected by antibodies recognizing any of their component parts, such as, but not limited to, those described herein.

In certain embodiments, over 90% of the described ASC, prior to incubation with inflammatory cytokines, where relevant, are positive for CD29, CD90, and CD54. “Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. In other embodiments, over 85% of the described cells are positive for CD73 and CD105; and over 65% of the described cells are positive for CD49. In yet other embodiments, less than 1% of the described cells are positive for CD14, CD19, CD31, CD34, CD39, CD45, HLA-DR, and GlyA; at least 30% of the cells are positive for CD200; less than 6% of the cells are positive for GlyA; and less than 20% of the cells are positive for SSEA4. In more specific embodiments, over 90% of the described cells are positive for CD29, CD90, and CD54; over 85% of the cells are positive for CD73 and CD105; and over 65% of the cells are positive for CD49. In still other embodiments, over 90% of the described cells are positive for CD29, CD90, and CD54; over 85% of the cells are positive for CD73 and CD105; over 65% of the cells are positive for CD49; less than 1% of the cells are positive for CD14, CD19, CD31, CD34, CD39, CD45, HLA-DR, GlyA; at least 30% of the cells are positive for CD200; less than 6% of the cells are positive for GlyA; and less than 20% of the cells are positive for SSEA4. In other embodiments, the ASC that have been incubated with inflammatory cytokines exhibit the aforementioned marker expression characteristics.

In other embodiments, each of CD73, CD29, and CD105 is expressed by more than 90% of the ASC, prior to incubation with inflammatory cytokines, where relevant. In still other embodiments, each of CD44, CD73, CD29, and CD105 is expressed by more than 90% of the cells. In yet other embodiments, each of CD34, CD45, CD19, CD14 and HLA-DR is expressed by less than 3% of the cells. In other embodiments, each of CD73, CD29, and CD105 is expressed by more than 90% of the cells, and each of CD34, CD45, CD19, CD14 and HLA-DR is expressed by less than 30% of the cells. In other embodiments, each of CD44, CD73, CD29, and CD105 is expressed by more than 90% of the cells, and each of CD34, CD45, CD19, CD14 and HLA-DR is expressed by less than 3% of the cells. In other embodiments, the ASC that have been incubated with inflammatory cytokines exhibit the aforementioned marker expression characteristics.

Alternatively or in addition, the ASC express the marker D7-fib, which is typically expressed on fibroblasts. Antibodies against D7-fib are commercially available from Acris Antibodies, Herford, Germany.

According to some embodiments, the ASC, prior to incubation with inflammatory cytokines, where relevant, express CD200, or, in other embodiments, lack expression thereof. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 30%, 35%, 40%, 45%, 50%, 55%, 60%, 65%, 70%, 75%, 80%, 85%, 90%, 92%, 94%, 95%, 96%, 97%, 98%, 99%, or 99.5% of the adherent cells express CD200. In other embodiments, the ASC that have been incubated with inflammatory cytokines exhibit the aforementioned marker expression characteristics.

According to some embodiments, greater than 50%, in other embodiments greater than 55%, in other embodiments greater than 60%, in other embodiments greater than 65%, in other embodiments greater than 70%, in other embodiments greater than 75%, in other embodiments greater than 80%, in other embodiments greater than 85%, in other embodiments greater than 90%, in other embodiments greater than 95%, in other embodiments greater than 96%, in other embodiments greater than 97%, in other embodiments greater than 98%, in other embodiments greater than 99% of the ASC do not express a marker selected from CD3, CD4, CD45, CD80, HLA-DR, CD11b, CD14, CD19, CD34, and CD79-alpha, or in other embodiments do not express 2 or more of these markers, or in other embodiments 3 or more of these markers, or in other embodiments 4 or more of these markers, or in other embodiments 5 or more of these markers, or in other embodiments 6 or more of these markers, or in other embodiments 7 or more of these markers, or in other embodiments 8 or more of these markers, or in other embodiments 9 or more of these markers, or in other embodiments all ten of these markers, prior to incubation with inflammatory cytokines, where relevant. In other embodiments, the ASC that have been incubated with inflammatory cytokines exhibit the aforementioned marker expression characteristics.

Additionally or alternatively, the ASC, prior to incubation with inflammatory cytokines, where relevant, secrete or express IL-6, eukaryotic translation elongation factor 2 (EEEF2), reticulocalbin 3, EF-hand calcium binding domain (RCN₂), and/or calponin 1 basic smooth muscle (CNN1). In more specific embodiments, greater than 50%, in other embodiments greater than 55%, in other embodiments greater than 60%, in other embodiments greater than 65%, in other embodiments greater than 70%, in other embodiments greater than 75%, in other embodiments greater than 80%, in other embodiments greater than 85%, in other embodiments greater than 90%, in other embodiments greater than 95%, in other embodiments greater than 96%, in other embodiments greater than 97%, in other embodiments greater than 98%, in other embodiments greater than 99%, of the cells express or secrete at least one, in other embodiments at least 2, in other embodiments at least 3, in other embodiments at least 4, in other embodiments all five of the aforementioned proteins.

In certain embodiments, the described cells have been transfected with one or more therapeutic factors, which may be, in certain embodiments, anti-tumor factors. In other embodiments, the cells have not been transfected with any exogenous genetic material.

In still other embodiments, the ASC may be allogeneic, or in other embodiments, the cells may be autologous. In other embodiments, the cells may be fresh or, in other embodiments, frozen (e.g., cryo-preserved).

Pharmaceutical Compositions

The described ASC, or CM derived therefrom, can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprises ASC or CM in combination with an excipient, e.g., a pharmacologically acceptable excipientIn further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of ASC mentioned herein, each of which is considered a separate embodiment.

Since non-autologous cells may in some cases induce an immune reaction when administered to a subject, several approaches may be utilized according to the methods provided herein to reduce the likelihood of rejection of non-autologous cells. In some embodiments, these approaches include either suppressing the recipient immune system or encapsulating the non-autologous cells in immune-isolating, semipermeable membranes before transplantation. In some embodiments, this may be done, in various embodiments, whether or not the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

Examples of immunosuppressive agents that may be used in the methods and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner (as detailed herein). Alternatively, one may administer the pharmaceutical composition locally, for example, via injection of the pharmaceutical composition directly into a tissue region of a patient, such as, in non-limiting embodiments, intratumoral administration. In other embodiments, the cells are administered intramuscularly, intravenously (IV), subcutaneously (SC), intratracheally, or intraperitoneally (IP), each of which is considered a separate embodiment. In still other embodiments, the pharmaceutical composition is administered intralymphatically, for example as described in U.S. Pat. No. 8,679,834 in the name of Eleuterio Lombardo and Dirk Buscher, which is hereby incorporated by reference.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

A typical dosage of the described ASC used alone ranges, in some embodiments, from about 10 million to about 500 million cells per administration, for a human subject. For example, the dosage can be, in some embodiments, 10, 20, 30, 40, 50, 60, 70, 80, 90, 100, 125, 150, 175, 200, 225, 250, 275, 300, 325, 350, 375, 400, 425, 450, 475, or 500 million cells or any amount in between these numbers. It is further understood that a range of ASC can be used including from about 10 to about 500 million cells, from about 100 to about 400 million cells, from about 150 to about 300 million cells. Accordingly, disclosed herein are therapeutic methods, the method comprising administering to a subject a therapeutically or prophylactically effective amount of ASC, wherein the dosage administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or, in other embodiments, between 150 million to 300 million cells. ASC, compositions comprising ASC, and/or medicaments manufactured using ASC can be administered, in various embodiments, in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections, or more.

Subjects

In certain embodiments, the subject tested or treated by the described methods and compositions is a human subject having a tumor. In other embodiments, the subject may be an animal subject having a tumor. In some embodiments, treated animals include domesticated animals and laboratory animals, e.g., non-mammals and mammals, for example non-human primates, rodents, pigs, dogs, and cats.

Additional objects, advantages, and novel features of the invention will become apparent to one ordinarily skilled in the art upon examination of the following Examples, which are not intended to be limiting. Additionally, each of the various embodiments and aspects of the invention as delineated hereinabove and as claimed in the claims section below finds experimental support in the following Examples.

### EXAMPLES

Reference is now made to the following Examples, which together with the above descriptions illustrate certain embodiments in a non-limiting fashion.

### Example 1

### Production and Culturing of Adherent Stromal Cells

Overview:

The manufacturing process for the cell product consisted of 2 stages:

Stage 1, the intermediate cell stock (ICS) production, contains the following steps:
1. Extraction of ASCs from the placenta.
2. 2-dimensional cell growth for up to 12 population doublings.
3. Cell concentration, formulation, filling and cryopreservation.
   Stage 2, the thawing of the ICS and further culture, contains the following steps:
1. 2-dimensional cell growth of the thawed ICS for up to 8 additional doublings.
2. 3-dimensional cell growth in bioreactor/s and harvest from bioreactor/s up to 10 additional doublings.
3. Downstream processing: cell concentration, washing, formulation, filling and cryopreservation.

The procedure included periodic testing of the growth medium for sterility and contamination.

### Production of ICS

### Step 1-1—Extraction of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled until the initiation of the extraction process.

Within 4 hours of the delivery, the placenta was placed with the maternal side facing upwards and was cut into pieces of approximately 1 cm³, which were washed thoroughly with isotonic buffer) containing gentamicin.
The washed pieces were incubated for 3 hours with collagenase and DNAse in isotonic buffer.
Culture medium (DMEM], 10% filtered FBS and L-Glutamine) supplemented with gentamicin, was added, and the digested tissue was coarsely filtered through a sterile stainless steel sieve and centrifuged.
The cells were suspended in culture medium, seeded in flasks, and incubated at 37° C. in a tissue culture incubator under humidified conditions supplemented with 5% CO₂.
After 2-3 days, cells were washed twice with Phosphate-Buffered Saline (PBS), and the culture medium was replaced.
Cells were incubated for an additional 4-5 days prior to the first passage.

### Step 1-2—Initial 2-Dimensional Culturing

Passage 1: Cells were detached using trypsin, centrifuged, and seeded at a culture density of 3±0.2×10³ cells/cm² in tissue culture flasks, in culture medium lacking gentamicin.
Subsequent Passages: When the culture reached 60-90% confluence, cells were passaged as described above.

### Step 1-3—Cell Concentration, Washing, Formulation, Filling and Cryopreservation

Following the final passage, the resulting cell suspension was centrifuged and resuspended in culture medium at a final concentration of 20-40×10⁶ cells/milliliter (mL). The cell suspension was diluted 1:1 with 2D Freezing Solution (20% DMSO, 80% FBS), and the cells were cryopreserved in 100/DMSO, 40% FBS, and 50% full DMEM. The temperature was reduced in a controlled rate freezer (1° C./min down to −80° C. followed by 5° C./min down to −120° C.), and the cells were stored in a liquid nitrogen freezer to produce the ICS.

### Production of Cell Product

### Step 2-1: Additional Two-Dimensional (2D) Cell Culturing

The ICS was thawed, diluted with culture medium, and cultured for up to 10 additional doublings, passaging when reaching 60-90% confluence, then were harvested for seeding in the bioreactor.

### Step 2-2: Three Dimensional (3D) Cell Growth in Bioreactor/s

From the cell suspension, 1 or 2 bioreactors were seeded. Each bioreactor contained FibraCel® carriers (New Brunswick Scientific) made of polyester and polypropylene, and culture medium. 170×10⁶ cells were seeded into each 2.8-liter bioreactor.

The culture medium in the bioreactor/s was kept at the following conditions: temp: 37±1° C., Dissolved Oxygen (DO): 70±10% and pH 7.4±0.2. Filtered gases (Air, CO₂, N₂ and O₂) were supplied as determined by the control system in order to maintain the target DO and pH values.

After seeding, the medium was agitated with stepwise increases in the speed, up to 150-200 RPM by 24 hours. Perfusion was initiated several hours after seeding and was adjusted on a daily basis in order to keep the glucose concentration constant at approximately 550 mg/liter.

Cell harvest was performed at the end of the growth phase (approximately day 6). Bioreactors were washed for 1 minute with pre-warmed sterile PBS, and cells were detached. The cells were found to be over 90% maternally-derived cells.

### Step 2-3: Downstream Processing: Cell Concentration, Washing, Formulation, Filling and Cryopreservation

In some experiments, the cell suspension underwent concentration and washing, using suspension solution (5% w/v human serum albumin [HSA] in isotonic solution) as the wash buffer, and diluted 1:1 with 3D-Freezing solution (20% DMSO v/v and 5% HSA w/v in isotonic solution) to a concentration of 10-20×10⁶ cells/ml. In some experiments, a 1:1 mixture of 2D Freezing Solution and full DMEM was used, and the cell concentration was 3-5×10⁶ cells/ml. The temperature of the vials was gradually reduced, and the vials were stored in a gas-phase liquid nitrogen freezer.

### Example 2

### Osteocyte and Adipose Differentiation Assays

Methods

Bone Marrow Adherent Cells—

Bone marrow (BM) adherent cells were obtained from aspirated sterna marrow of hematologically healthy donors undergoing open-heart surgery or BM biopsy. Marrow aspirates were diluted 3-fold in HBSS) and subjected to Ficoll-Hypaque (Robbins Scientific Corp. Sunnyvale, Calif.) density gradient centrifugation. Thereafter, marrow mononuclear cells (<1.077 gm/cm³) were collected, washed 3 times in HBSS, and resuspended in growth media [DMEM (Biological Industries, Beit Ha'emek, Israel) supplemented with 10% FCS (GIBCO BRL), 10⁻⁴ M mercaptoethanol (Merck, White House Station, N.J.), Pen-Strep-Nystatin mixture (100 U/ml:100 μg/ml:1.25 un/ml; Beit Ha'Emek), 2 mM L-glutamine (Beit Ha'Emek)]. Cells from individual donors were incubated separately in tissue culture flasks (Corning, Acton, Mass.) at 37° C. (5% CO₂) with weekly change of culture media. Cells were passaged every 3-4 days using 0.25% trypsin-EDTA (Beit Ha'Emek). Following 2-40 passages, when reaching 60-80% confluence, cells were collected for analysis.

TABLE 1
Osteogenesis medium components

| Component | Stock conc. | Amount | | Final conc. | |
|---|---|---|---|---|---|
| DMEM low glucose (Invitrogen, | | 8.7 | ml | 87% | |
| Gibco) | | | | | |
| Serum (heat inactivated) | | 1 | ml | 10% | |
| Dexamethasone | 1 mM | 1 | μl | 0.1 | μM |
| Ascorbic Acid-2-Phosphate | 0.1M | 20 | μl | 0.2 | mM |
| solution | | | | | |
| Glycerol-2-Phosphate Solution | 1M | 100 | μL | 10 | mM |

| | | | | |
|---|---|---|---|---|
| L-glutamine | X 100 | 100 | μl | X 1 |
| Pen & Strep | X 100 | 100 | μl | X 1 |

Induction of Osteogenesis

Placenta-derived cells or BM-derived cells were plated (200,000 cells per well) in 1 ml growth medium comprising DMEM (Invitrogen, Gibco), 10% FCS (Invitrogen, Gibco), 2 Mm L-glutamine (Sigma-Aldrich), 45 μg/ml Gentamicin-IKA (Teva Medical) and 0.25 μg/ml Fungizone (Invitrogen, Gibco) in wells coated with a coating mixture containing 12 μg/ml vitronectin and 12 μg/ml collagen, which was provided with the Millipore Mesenchymal Stem Cell Osteogenesis Kit. Cells were grown until 100% confluent (typically overnight) before adding osteogenic differentiation medium.

On differentiation day 1, growth medium was aspirated and replaced with 1 ml osteogenesis induction medium, which was replaced with fresh medium every 2-3 days for 14-17 days. Osteocytes were fixed and stained with Alizarin Red Solution.

In other experiments, a modified osteogenesis induction medium was used, having the components listed in Table 2, including Vitamin D, for 26 days.

TABLE 2
Modified osteogenesis medium components

| Component | Stock conc. | Amount | | Final conc. | |
|---|---|---|---|---|---|
| DMEM high glucose (Biological | | 8.7 | ml | 87% | |
| Industries, Bet HaEmek, Israel) | | | | | |
| L-glutamine | X 100 | 100 | μl | X 1 | |
| Serum (heat inactivated) | | 1 | ml | 10% | |
| Dexamethasone (Chemicon) | 10 mM | 10 | μl | 10 | μM |
| Ascorbic Acid-2-Phosphate | 0.1M | 20 | μl | 0.2 | mM |
| solution (Chemicon) | | | | | |
| Glycerol-2-Phosphate Solution | 1M | 100 | μL | 10 | mM |
| (Chemicon) | | | | | |
| Vitamin D (Sigma) | 10 μM | 10 | μL | 10 | nM |

| | | | | |
|---|---|---|---|---|
| Gentamycin | X 100 | 100 | μl | X 1 |
| (Biological Industries, | | | | |
| Bet HaEmek, Israel) | | | | |

Induction of Adipogenesis

Adipogenesis was carried out according to the instructions provided with the Chemicon Adipogenesis Kit (cat no. scr020, Millipore, Mass., USA)

Adipogenesis Induction Medium

Adipogenesis induction and maintenance medium were freshly prepared prior to every medium exchange, using the components depicted in Tables 3 and 4, below.

TABLE 3
Adipogenesis induction medium components

| Component | Stock conc. | | Amount | | Final conc. | |
|---|---|---|---|---|---|---|
| DMEM low glucose (Biological | | | 4.4 | ml | 90% | |
| Industries, Bet HaEmek, Israel) | | | | | | |
| Serum (heat inactivated) | | | 0.5 | ml | 10% | |
| Dexamethasone (Sigma) | 10 | mM | 0.5 | μl | 1 | μM |
| IB MX (Sigma) | 0.5M | | 5 | μl | 0.5 | mM |
| Insulin (Sigma) | 10 | mg/ml | 5 | μL | 10 | μg/ml |
| Indomethacin (Sigma) | 10 | mM | 50 | μl | 100 | μM |

| | | | | |
|---|---|---|---|---|
| Pen & Strep | X 100 | 50 | μl | X 1 |

TABLE 4
Adipogenesis maintenance medium components

| Component | Stock conc. | Amount | | Final conc. |
|---|---|---|---|---|
| DMEM low glucose | | 4.4 | ml | 90% |
| Serum (heat inactivated) | | 0.5 | ml | 10% |
| Insulin | 10 mg/ml | 5 | μL | 10 μg/ml |
| Pen & Strep | X 100 | 50 | μl | X 1 |

Cell Growth

Placenta-derived or BM-derived cells were plated (200,000 cells per well) in 1 ml growth medium comprising DMEM (Invitrogen, Gibco), 10% FCS (Invitrogen, Gibco), 2 mM L-glutamine (Sigma-Aldrich), 45 μg/ml Gentamicin-IKA (Teva Medical) and 0.25 μg/ml Fungizone (Invitrogen, Gibco) and were grown until 100% confluent (typically overnight) before initiating adipogenesis differentiation.

On differentiation day 1, growth medium was aspirated and replaced with 1 ml adipogenesis induction medium, which was replaced with fresh induction or maintenance medium every 2-3 days for a total of 25 days, according to the schedule in Table 5.

TABLE 5
Adipogenesis differentiation schedule

| Day | Medium |
|---|---|
| 1 | Adipogenesis Induction medium |
| 3 | Adipogenesis Induction medium |
| 5 | Adipogenesis Induction medium |
| 7 | Adipogenesis Maintenance medium |
| 9 | Adipogenesis Induction medium |
| 11 | Adipogenesis Induction medium |
| 13 | Adipogenesis Induction medium |
| 15 | Adipogenesis Maintenance medium |
| 17 | Adipogenesis Induction medium |
| 19 | Adipogenesis Induction medium |
| 21 | Adipogenesis Induction medium |

On day 25, adipocytes were fixed and stained with oil red solution.

Modified Adipogenesis Induction Medium

The modified adipogenesis induction medium contained the components depicted in Table 6, and was used for a total of 26 days.

TABLE 6
Modified adipogenesis induction medium components

| Component | Stock con | | Amount | | Final conc. | |
|---|---|---|---|---|---|---|
| DMEM low glucose | | | 4.4 | ml | 90% | |
| Serum (heat inactivated) | | | 0.5 | ml | 10% | |
| Dexamethasone (Sigma) | 1 | mM | 5 | μl | 1 | μM |
| IBMX (Sigma) | 0.5M | | 5 | μl | 0.5 | mM |
| Insulin (Sigma) | 10 | mg/ml | 5 | μL | 10 | μg/ml |
| Indomethacin (Sigma) | 10 | mM | 200 | μl | 200 | μM |
| Gentamycine | | | 10 | μl | | |
| (Biological Industries) | | | | | | |

Results

Osteocyte Induction.

Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation.

Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte Induction.

Adipocyte differentiation of placenta- or BM-derived adherent cells in adipocyte induction medium resulted in differentiation of over 50% of the BM-derived cells, as demonstrated by positive oil red staining and by typical morphological changes (e.g. accumulation of oil droplets in the cytoplasm). In contrast, none of the placental-derived cells differentiated into adipocytes.

Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 3

### Marker Expression on Adherent Stromal Cells

### Methods (Examples 3-4)

FACS analysis of membrane markers was performed by staining cells with monoclonal antibodies (MAbs). 400,000-600,000 cells were suspended in 1 ml flow cytometer buffer in a 5 ml test tube and incubated for 15 minutes at room temperature (RT), in the dark, with each of the following MAbs: PE-conjugated anti-human CD29 MAb (Becton Dickinson), PE-conjugated anti human CD73 MAb (Becton Dickinson), PE-conjugated anti human CD105 MAb (Becton Dickinson), PE-conjugated anti human CD90 MAb (Becton Dickinson), PE-conjugated anti-human CD45 MAb (Becton Dickinson), PE-conjugated anti-human CD19 MAb (Becton Dickinson), PE-conjugated anti human CD14 MAb (Becton Dickinson), PE-conjugated anti human HLA-DR MAb (Becton Dickinson), PE-conjugated anti human CD34 MAb (Becton Dickinson), PE-conjugated anti human CD31 MAb (Becton Dickinson), PE-conjugated anti-human CD200 MAb (Becton Dickinson), Isotype IgG2beta PE-conjugated (Becton Dickinson), Isotype IgG1 alpha PE-conjugated (Becton Dickinson); and anti-CD106, anti-CD54, anti-CD56, anti-CD49d, anti-glyA, and anti-CD39, all PE-conjugated and from Becton Dickinson; Alexa Fluor®-conjugated anti-SSEA4 (eBioscience), and IgG3 kappa isotype control (Biolegend).

Cells were washed twice with flow cytometer buffer, resuspended in 400 microliters (mcl) flow cytometer buffer, and analyzed by flow cytometry.

Results

Expression of Cellular Markers on Isolated Cells—

the surface antigens expressed by the isolated cells were examined using monoclonal antibodies. The cells expressed CD73, CD29, and CD105, and did not express the markers CD34, CD45, CD19, CD14, and HLA-DR. More specifically, all the positive markers were expressed by more than 90% of the cells, and all the negative markers were expressed by less than 3% of the cells.

Furthermore, the cells did not express endothelial markers as shown by negative staining for the two endothelial markers CD31 and KDR. However, expression of a fibroblast-typical marker, D7-fib, was evident.

### Example 4

### Treatment of ASC with Pro-Inflammatory Cytokines During 3D Culturing

Methods

General Experimental Protocol.

ASC were obtained from the placenta and cultured under 2D conditions, then under 3D conditions, and were then harvested, all as described in Example 1, with the following deviation: One day before the end of the 3D culture (typically on day 5 or 6), the medium was replaced with DMEM, with or without the addition of 10 nanograms/milliliter (ng/ml) Tumor Necrosis Factor alpha (TNF-alpha), 10 ng/ml Interferon-Gamma (IFN-g), and/or 10% FBS (see Table 7), and the bioreactor was incubated in batch mode (or, in selected experiments, in perfusion mode) for an additional day. Levels of secreted cytokines were measured in the bioreactor medium, using the RayBio® Human Cytokine Array kit.

Hypoxic Incubation.

1×10⁶ thawed ASC were seeded in 2 ml DMEM medium. After 24 hours (hr), the medium was replaced with EBM-2 medium (Lonza Group Ltd, Basel, Switzerland), and cells were incubated under hypoxic conditions (1% O₂) for an additional 24 hr, after which the conditioned media was collected.

TABLE 7
Incubation conditions that were tested

| Designation | Cytokines | FBS |
|---|---|---|
| 1 | None | NO |
| 2 | None | YES |
| 3 | TNF | NO |
| 4 | TNF | YES |
| 5 | TNF + IFN | NO |
| 6 | TNF + IFN | YES |

In other experiments, levels of secreted cytokines were measured in the conditioned medium (CM) from a hypoxic incubation, as described above.

Quantitative Detection of Secreted Proteins:

IL-6 was quantitatively measured using the human IL-6 immunoassay Quantikine® ELISA kit (R&D Systems). VEGF was quantitatively measured using the Human VEGF immunoassay Quantikine® kit (R&D Systems).

Results

In a series of experiments testing various conditions side-by-side, ASC were incubated in a bioreactor as described in the previous Examples. On the last day of the bioreactor incubation, the medium was replaced by medium containing or lacking added TNF-alpha and/or IFN-gamma, in the presence or absence of FBS. VEGF and 1-6 secretion were measured in the bioreactor medium by ELISA. Inclusion of TNF-alpha significantly increased secretion of VEGF, whether or not IFN-gamma was present (Table 8).

TABLE 8
Secretion of VEGF (picograms/ml [pg/ml])
by ASC under various conditions

| Expt. | | | VEGF in | VEGF in bioreactor |
|---|---|---|---|---|
| # | Cytokines | FBS | CM/RPD* | medium/RPD* |
| 1 | TNF + IFN | NO | 619/3 | 195/3 |
| | None | NO | 274/7 | 65/0 |
| 2 | TNF + IFN | NO | 7540/1 | 151/3 |
| | None | NO | 3266/4 | 140/3 |
| 3 | TNF + IFN | YES | 371/3 | 1749/2 |
| | TNF | YES | 370/10 | 1128/5 |
| 4 | TNF + IFN | YES | NT (not tested) | 373/2 |
| | TNF | YES | NT | 348/8 |
| 5 | TNF + IFN | NO | 732 ± 20** | (not performed) |
| | None | NO | 650 ± 46** | (not performed) |

*In this table and throughout the document, except where indicated otherwise, RPD refers to the percentage difference between duplicate samples in the ELISA.
**Indicated number is the standard deviation.

In the same experiment, inclusion of TNF-alpha significantly increased IL-6 secretion, which was further increased by IFN-gamma, as shown in Table 9.

TABLE 9
Secretion of IL-6 (picograms/ml [pg/ml])
by ASC under various conditions

| Expt. | | | | |
|---|---|---|---|---|
| # | Cytokines | FBS | IL-6 in CM | RPD |
| 1 | TNF + IFN | NO | 77 | 2 |
| | None | NO | 10 | 2 |
| 2 | TNF + IFN | NO | 509* | 1 |
| | None | NO | 40 | 4 |
| 3 | TNF + IFN | YES | 380 | 0 |
| | TNF | YES | 92 | |

*above calibration curve.

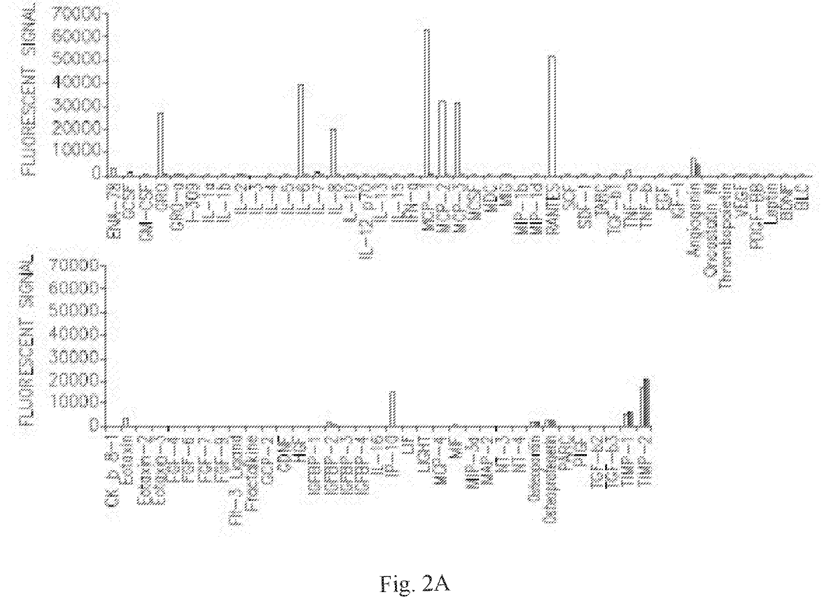

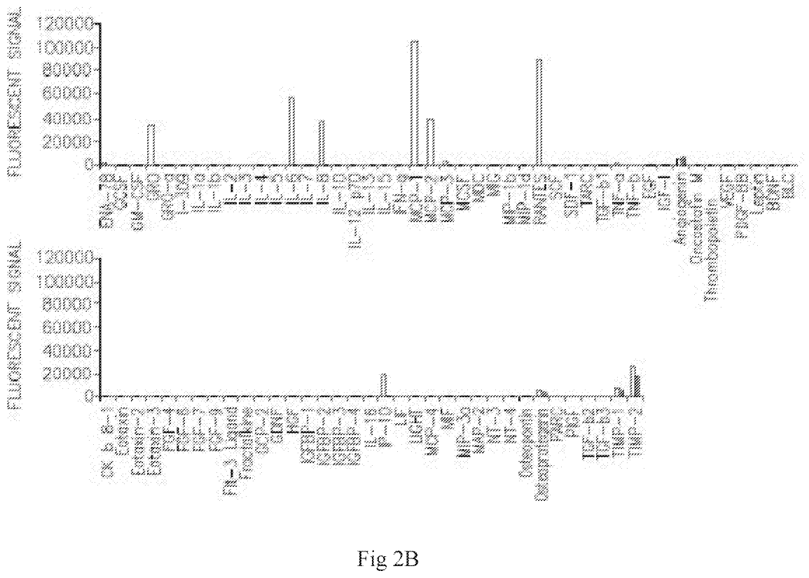

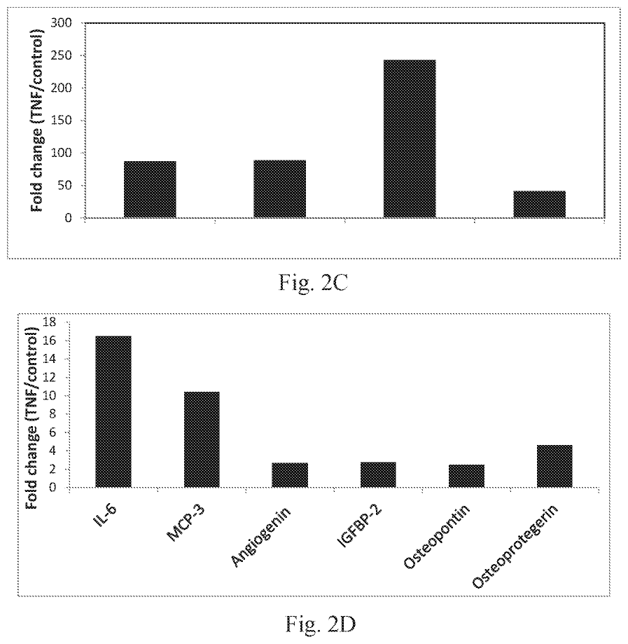

Expression of a panel of factors in the bioreactor media of Experiments 1-2 (see Tables 8-9), all performed in the absence of serum, was measured by a fluorescence-based cytokine array assay, revealing the increased expression of several factors, including GRO, IL-6, IL-8, MCP-1, MCP-2, MCP-3, RANTES, and IP-10 (Experiments 1-2 are shown in FIGS. 2A-B, respectively). In another experiment, TNF-alpha alone was compared to medium without cytokines (also in the absence of serum), showing increased expression of GRO, IL-8, MCP-1, RANTES, and, to a lesser extent, IL-6, MCP-3, Angiogenin, Insulin-like Growth Factor Binding Protein-2 (IGFBP-2), Osteopontin, and Osteoprotegerin (FIGS. 2C-D).

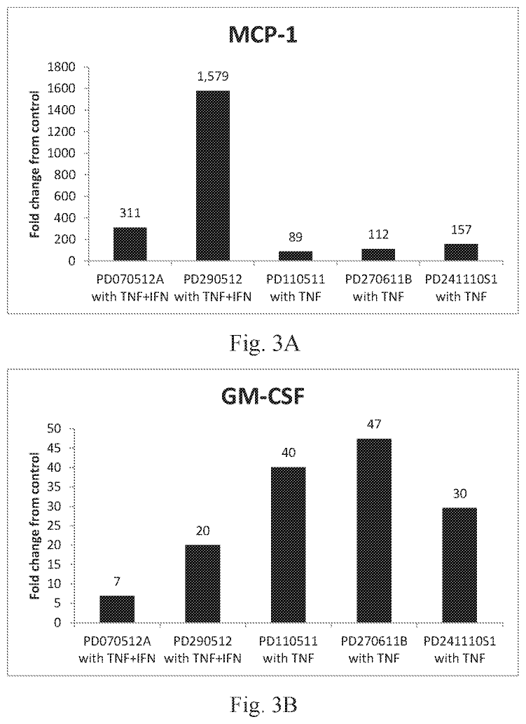

Increased expression of MCP-1 and GM-CSF in the bioreactor media was verified by quantitative ELISA in several experiments, all performed in the absence of serum. The results showed that TNF-alpha+IFN-gamma was more potent than TNF-alpha alone for MCP-1 induction (FIG. 3A), while TNF-alpha alone appeared to be slightly superior for GM-CSF induction (FIG. 3B). The cytokine concentrations and fold-changes relative to control medium (containing no cytokines) from the TNF-alpha+IFN-gamma trial are shown in Table 10 below.

TABLE 10
MCP-1 and GM-CSF concentrations in bioreactor medium

| Expt. | | MCP-1 (pg/ml) | | GM-CSF (pg/ml) | |
|---|---|---|---|---|---|
| No. | Conditions | (fold-increase) | | (fold-increase) | |
| 1 | TNF + IFN | 6365.4 | (311) | 6.32 | (6.9) |
| | None | 20.5 | | 0.91 | |
| 2 | TNF + IFN | 9063.7 | (1579) | 13.09 | (20.0) |

| | | |
|---|---|---|
| None | 5.8 | 0.65 |

The induction of several other factors, over several experiments utilizing TNF-alpha+IFN-gamma or TNF-alpha alone (all in the absence of serum), was detected by the aforementioned cytokine array. A number of proteins were consistently upregulated, as depicted in Table 11 below.

TABLE 11
Fold-enrichment (relative to no-cytokine control cells)
of selected proteins upon incubation with TNF-alpha +/−
IFN-gamma. Only fold-changes greater than 2 are depicted

| Condition/Expt. | TNF + IFN/ | TNF + IFN/ | TNF alone/ |
|---|---|---|---|
| No. | expt. 1 | expt. 2 | expt. 6 |
| Proteins | | | |
| ENA-78 | 13.0 | 11.4 | |
| GCSF | 4.6 | 3.3 | |
| GM-CSF | 3.7 | 3.1 | |
| GRO | 57.8 | 102.7 | 87 |
| GRO-a | 2.9 | 2.5 | |
| IL-2 | 3.8 | 3.2 | |
| IL-6 | 199.2 | 281.4 | 16.5 |
| IL-7 | 4.6 | 2.5 | |
| IL-8 | 32.6 | 80.5 | 88.7 |
| IL-10 | 3.2 | 3.5 | |
| IFN- g | 2.9 | 2.8 | |
| MCP-1 | 88.3 | 529.3 | 243.3 |
| MCP-2 | 88.3 | 198.5 | |
| MCP-3 | 160.7 | 18.0 | 10.4 |
| MIG | 158.2 | 3.2 | |
| RANTES | 4.4 | 452.1 | 41.3 |
| TGF-b1 | 256.7 | 3.5 | |
| VEGF | 4.3 | | |
| Eotaxin | 17.6 | 2.1 | |
| IGFBP-2 | 2.3 | | 2.8 |
| IP-10 | 75.0 | 94.7 | |
| MIF | 3.0 | 2.9 | |
| Angiogenin | | | 2.7 |
| Osteopontin | | | 2.5 |
| Osteoprotegerin | | | 4.6 |

### Example 5

### The Effect of Serum on Pro-Inflammatory Cytokine Treatment of ASC During 3D Culturing

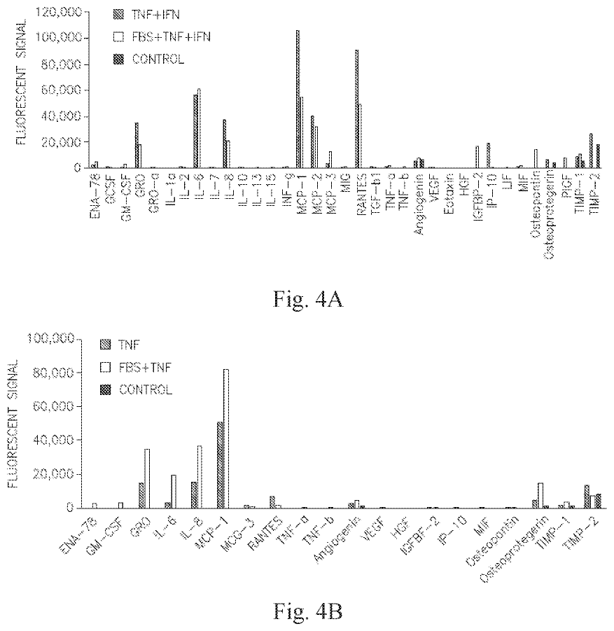

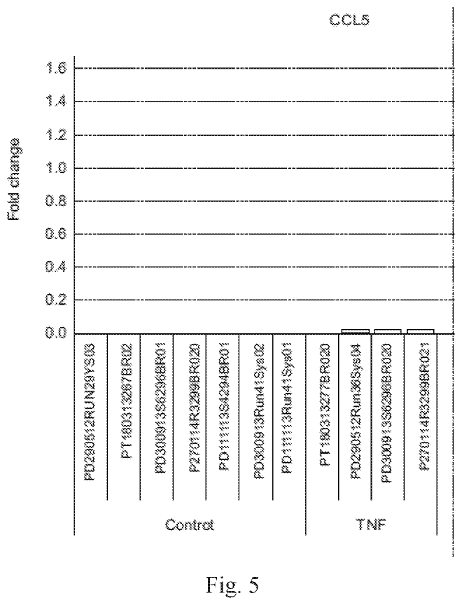

The next experiment examined the effect of FBS on induction of the aforementioned panel of factors by TNF-alpha+IFN-gamma (FIG. 4A) or TNF-alpha alone (FIG. 4B). A similar set of major proteins was induced in the presence or absence of FBS. In the case of TNF-alpha alone, IL-6 appeared to be induced much more strongly in the presence of FBS than in its absence.

### Example 6

### Quantitative Rantes ELISA on Pre-Treated ASC

ASC were incubated with 10 ng/ml TNF-alpha, alone or in combination with 10 ng/ml IFN-gamma, as described for Example 6. The cells were cryopreserved, then thawed, and then 5×10⁵ cells were seeded in DMEM supplemented with 10% FBS and incubated under standard conditions. After 24 hours, the medium was replaced with 1-ml serum-free medium, and the cells were incubated another 24 hours under normoxic conditions. The medium was removed and assayed for RANTES secretion by ELISA, using the Quantikine® ELISA Human CCL5/RANTES kit (R&D Systems). The TNF-alpha+IFN-gamma-treated cells had sharply upregulated RANTES secretion compared to the other groups (Table 12).

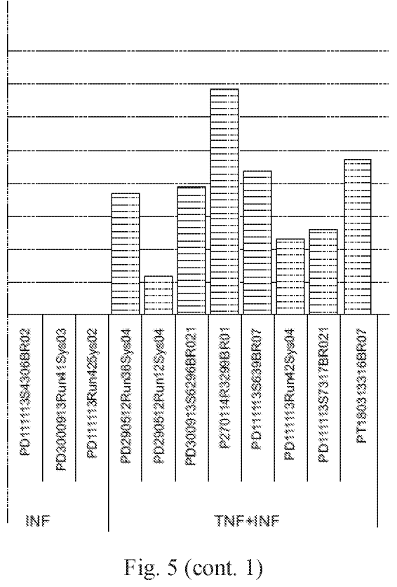

In a similar experiment, TNF-alpha+IFN-gamma treatment was tested in parallel with TNF-alpha alone, IFN-gamma alone, or no treatment. The average RANTES expression was more than 10-fold higher in the TNF-alpha+IFN-gamma-treated cells than any other group (FIG. 5).

TABLE 12
RANTES concentrations in culture medium

| Expt. | | RANTES | Standard |
|---|---|---|---|
| No. | Conditions | conc. | dev. |
| 5 | No cytokines, no serum | 0 | 0 |
| 7 | No cytokines, serum. | 2 | 1 |
| 8 | No cytokines, serum | 0 | 0 |
| 5 | TNF-alpha, no serum | 76 | 2 |
| 7 | TNF-alpha, serum. | 591 | 20 |
| 8 | IFN-gamma + TNF-alpha + serum. | 3232* | 83 |

*Out of calibration curve.

### Example 7

### Marker Phenotype of ASC Treated with Inflammatory Cytokines

The marker phenotype of the ASC that had been pre-treated with pro-inflammatory cytokines was examined over several experiments. Consistently, the cells were over 90% positive for CD29, CD90, and CD54; over 85% positive for CD73 and CD105; and over 65% positive for CD49. Additionally, the cells were less than 1% positive for CD14, CD19, CD31, CD34, CD39, CD45, and HLA-DR; less than 3% were positive for CD200; less than 6% were positive for GlyA; and less than 20% were positive for SSEA4.

### Example 8

### Effect of CM on Tumor Cell Replication and Survival

Methods

CM Production:

Bioreactor incubations were performed as described in Examples 1 and 6. 500,000 post-bioreactor cells were seeded in multi-well plates in 4 ml DMEM supplemented with 2 mM L-glutamine and 10% FBS, in some cases with the addition of 40 ng/well IFN-gamma. After 24 hours, the medium was aspirated, the cells were washed, and RPMI (with or without 10% FBS) was added. After a 24-hr incubation, the medium was collected and centrifuged, and 5% FBS was added to the medium.

Anti-Cancer Assay.

59 cell lines were grown in medium (RPMI, with 10% FBS, 2 mM L-alanyl-L-Glutamine, and 1 mM Sodium Pyruvate) and seeded in the above medium, with the addition of 10% FBS, to form spheroids, in multi-well 3D plates (Elplasia™ plates, which contain micro-spaces on the surface that allow cells to self-assemble) pre-coated with polyhydroxyethylmethacrylate (pHEMA). CM was serially diluted 2-fold and assayed over several concentrations in triplicate. CM, neat or diluted 1:2, 1:4, or 1:8, was added 24 hours post seeding in a volume of 25 μL and was exchanged every 3 days. Controls (positive and negative) were included for every cell line. Cells were lysed and analyzed using a CellTiter-Glo® Cell Viability Assay, to determine the effects of the CM on the viability and replication of the cells. An inhibition of 20-40% relative to vehicle was statistically significant relative to the standard deviations and was defined as partial inhibition, while an inhibition of 40% or more was defined as inhibition.

Results

ASC, either maternal or mixed maternal/fetal, were produced in a bioreactor and used to prepare conditioned media (CM). CM was prepared from 4 batches of ASC, some of which were subjected to treatment prior to or during CM production, as set forth in Table 13. The CM was tested for the ability to inhibit replication of various cancer cell lines.

TABLE 13
Tested cell lines

| | | FBS on | |
|---|---|---|---|
| Group | Composition | last day? | Other special treatment |
| 1 | Maternal | No | TNF-alpha/IFN-gamma on last |
| | | | day of bioreactor incubation as |
| | | | described in Example 6 |
| 2 | maternal/fetal | No | None |
| 3 | maternal/fetal | No | IFN-alpha present on first day |
| | | | of CM production |
| 4 | maternal/fetal | Yes | None |

The tested cell lines are shown in Table 14 below.

TABLE 14
Cell lines used for anti-cancer testing

| Cell Line | ATCC Cat. # | Cancer Type | Organ | Organ Notes |
|---|---|---|---|---|
| 22Rv1 | CRL-2505 | Prostate carcinoma | Prostate | |
| 647-V | ACC-414 | Urothelial bladder carcinoma | Bladder | |
| 769-P | CRL-1933 | Renal cell adenocarcinoma | Kidney | clear cell renal |
| | | | | cell carcinoma |
| 786-O | CRL-1932 | Renal cell adenocarcinoma | Kidney | clear cell renal |
| | | | | cell carcinoma |
| A-498 | HTB-44 | Renal cell carcinoma | Kidney | |
| A549 | CCL-185 | Non small cell carcinoma | Lung | |
| ACHN | CRL-1611 | Renal cell adenocarcinoma | Kidney | |
| AGS | CRL-1739 | Gastric adenocarcinoma | Stomach | |
| AsPC-1 | CRL-1682 | Pancreatic adenocarcinoma | Pancreas | Ductal |
| | | | | carcinoma |
| BT474 | HTB-20 | Breast ductal carcinoma | Breast | Breast/duct; |
| | | | | Mammary gland |
| C32 | CRL-1585 | Malignant melanoma | Skin | |
| C3A | CRL-10741 | Hepatocellular carcinoma | Liver | |
| Cal 27 | CRL-2095 | Squamous cell carcinoma | Head/Neck | Head and Neck |
| | | | | (tongue) |
| CAL-62 | ACC 448 | Thyroid anaplastic carcinoma | Thyroid | |
| Calu-6 | HTB-56 | Lung anaplastic carcinoma | Lung | |
| CHL-1 | CRL-9446 | Melanoma | Skin | |
| Colo 205 | CCL-222 | Colorectal adenocarcinoma | Colon/Rectum | Colon/GI |
| Colo 320 | CCL-220.1 | Colorectal adenocarcinoma; | Colon/Rectum | Colon |
| HSR | | Dukes' type C | | |
| COLO | CRL-1974 | Melanoma; Fibroblast | Skin | |
| 829 | | | | |
| DBTRG- | CRL-2020 | Astrocytoma | Brain | |
| 05MG | | | | |
| DLD-1 | CCL-221 | Colorectal adenocarcinoma | Colon/Rectum | Dukes' type C, |
| | | | | colorectal |
| | | | | adenocarcinoma |
| DU 145 | HTB-81 | Prostate carcinoma | Prostate | Prostate; derived |
| | | | | from metastatic |
| | | | | site: brain |
| ES-2 | CRL-1978 | Ovarian clear cell carcinoma | Ovary | |
| FaDu | HTB-43 | Hypopharyngeal squamous | Pharynx | |
| | | cell carcinoma | | |
| HCC1395 | CRL-2324 | Breast carcinoma | Breast | Mammary |
| | | | | gland, breast |
| HCT 116 | CCL-247 | Colorectal carcinoma | Colon/Rectum | Colon |
| HCT-15 | CCL-225 | Colorectal adenocarcinoma; | Colon/Rectum | Colon |
| | | Dukes' type C | | |
| Hela | CCL-2 | Adenocarcinoma | Cervix | Female GU |
| | | | | (Cervix) |
| Hep 3B2.1-7 | HB-8064 | Hepatocellular carcinoma | Liver | |
| Hep G2 | HB-8065 | Hepatocellular carcinoma | Liver | |
| HT-1376 | CRL-1472 | Urinary bladder carcinoma | Bladder | Transitional cell |
| | | | | carcinoma |
| HT-29 | HTB-38 | Colorectal adenocarcinoma | Colon/Rectum | Colon |
| Huh 7 | Huh7 | Hepatocellular carcinoma | Liver | |
| J82 | HTB-1 | Urinary bladder transitional | Bladder | |
| | | cell carcinoma | | |
| LNCaP | CRL-1740 | Prostate adenocarcinoma; | Prostate | |
| clone | | metastatic | | |
| FGC | | | | |
| LS 174T | CL-188 | Colorectal adenocarcinoma; | Colon/Rectum | Colon |
| | | Dukes' type B | | |
| MCF7 | HTB-22 | Breast adenocarcinoma | Breast | Breast; |
| | | | | mammary |
| | | | | gland, derived |
| | | | | from metastatic |
| | | | | site: pleural |
| | | | | effusion |
| MDA- | HTB-26 | Breast adenocarcinoma | Breast | Breast; |
| MB-231 | | | | mammary |
| | | | | gland, derived |
| | | | | from metastatic |
| | | | | site: pleural |
| | | | | effusion |
| MDA- | HTB-131 | Breast carcinoma; metastatic | Breast | Breast; |
| MB-453 | | | | mammary |
| | | | | gland, derived |
| | | | | from metastatic |
| | | | | site: pericardial |
| | | | | effusion |
| MES-SA | CRL-1976 | Uterine sarcoma | Uterus | |
| Mia | CRL-1420 | Pancreatic carcinoma | Pancreas | Ductal |
| PaCa-2 | | | | carcinoma |
| NCI- | CRL-5859 | Lung adenocarcinoma | Lung | Lung, derived |
| H1792 | | | | from metastatic |
| | | | | site: pleural |
| | | | | effusion |
| NCI-H23 | CRL-5800 | Lung adenocarcinoma, NSCL | Lung | |
| NCI-H358 | CRL-5807 | Bronchioalveolar carcinoma, | Lung | Lung; |
| | | NSCL | | Bronchiole |
| NCI-H460 | HTB-177 | Lung carcinoma; large cell | Lung | Lung; plueral |
| | | | | effusion |
| PC-3 | CRL-1435 | Prostate adenocarcinoma | Prostate | Prostate; derived |
| | | | | from metastatic |
| | | | | site, bone |
| RD | CCL-136 | Rhabdomyosarcoma | Muscle | |
| SK-MEL-3 | HTB-69 | Melanoma | Skin | Skin; derived |
| | | | | from Metastatic |
| | | | | Site: lymph |
| | | | | node |
| SK-N-AS | CRL-2137 | Neuroblastoma | Brain | Brain; dervied |
| | | | | from metastatic |
| | | | | site, bone |
| | | | | marrow |
| SK-OV-3 | HTB-77 | Ovarian adenocarcinoma | Ovary | Ovary; ascites |
| SNU-449 | CRL-2234 | Hepatocellular carcinoma; | Liver | |
| | | grade II-III/IV | | |
| SW1088 | HTB-12 | Astrocytoma | Brain | |
| SW48 | CCL-231 | Colorectal adenocarcinoma; | Colon/Rectum | Colon |
| | | Dukes' type C, grade IV | | |
| SW480 | CCL-228 | Colorectal adenocarcinoma; | Colon/Rectum | Colon |
| | | Dukes' type B | | |
| SW620 | CCL-227 | Colorectal adenocarcinoma | Colon/Rectum | Colon; derived |
| | | | | from metastatic |
| | | | | site: lymph node |
| T24 | HTB-4 | Carcinoma | Bladder | |
| T-47D | HTB-133 | Ductal carcinoma | Breast | Mammary |
| | | | | gland; derived |
| | | | | from metastatic |
| | | | | site: pleural |
| | | | | effusion |
| U-87 MG | HTB-14 | Astrocytoma | Brain | CNS |
| UM-UC-3 | CRL-1749 | Urinary Bladder carcinoma; | Bladder | |
| | | transitional cell | | |

The below analysis focuses on experimental Group 1, which received CM from ASC treated with TNF-alpha/IFN-gamma. Overall:
12 cell lines were inhibited (<60% Proliferation) by Group 1.
14 cell lines were partially inhibited (60-80% Proliferation) by Group 1.
28 cell lines were not inhibited by the CM.
4 cell lines were partially stimulated (120-140% Proliferation) by Group 1.
1 cell line was stimulated (>140% Proliferation) by Group 1.

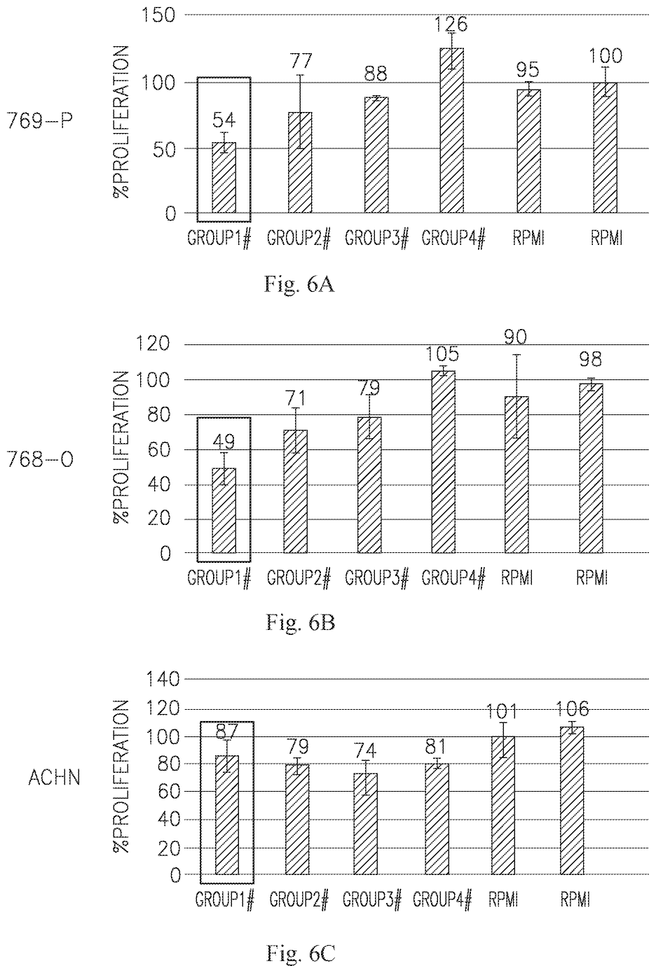

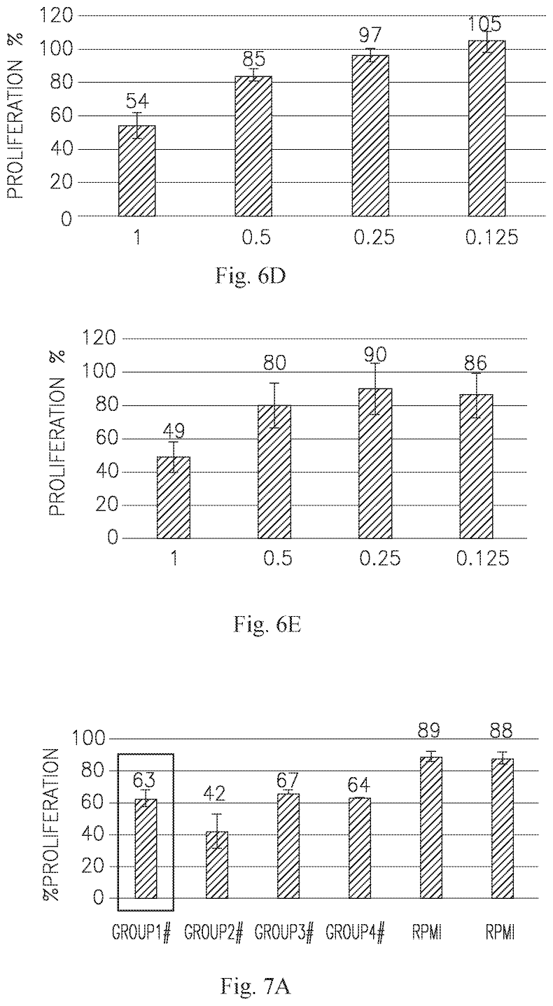

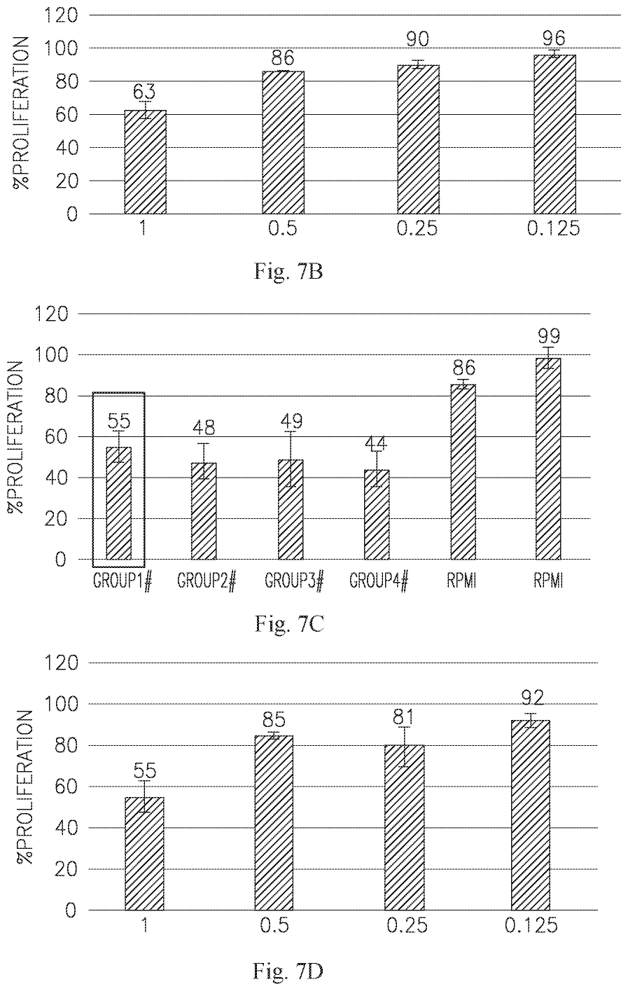

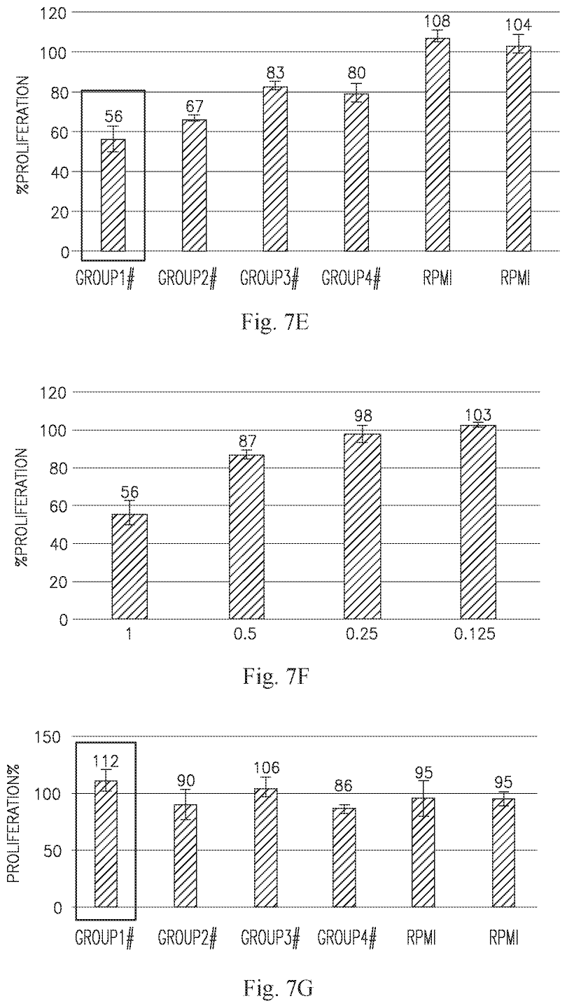

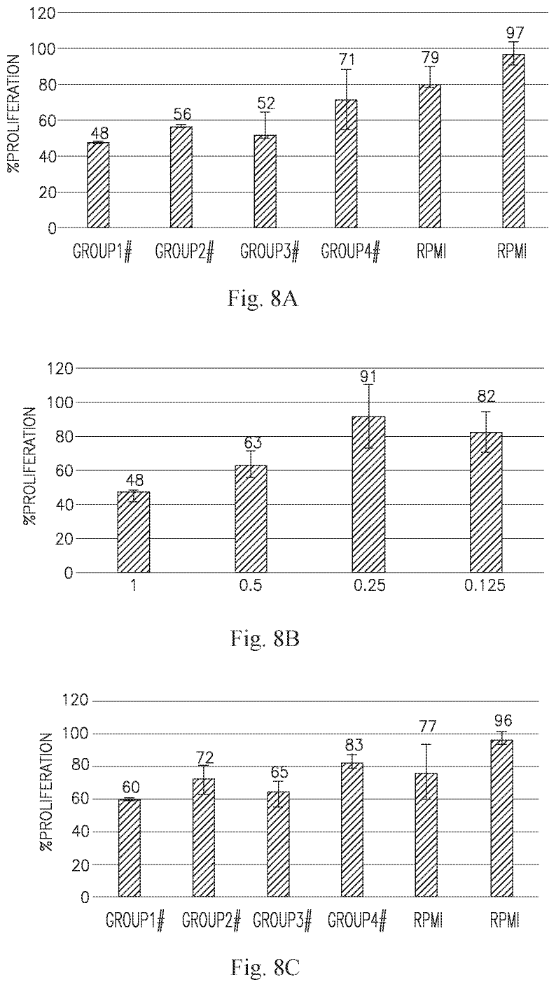

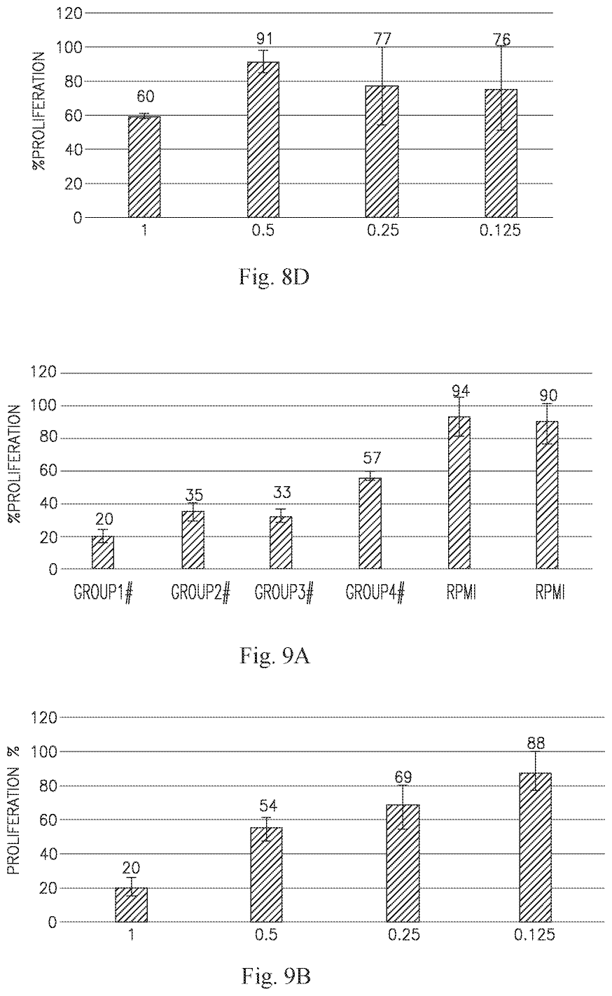

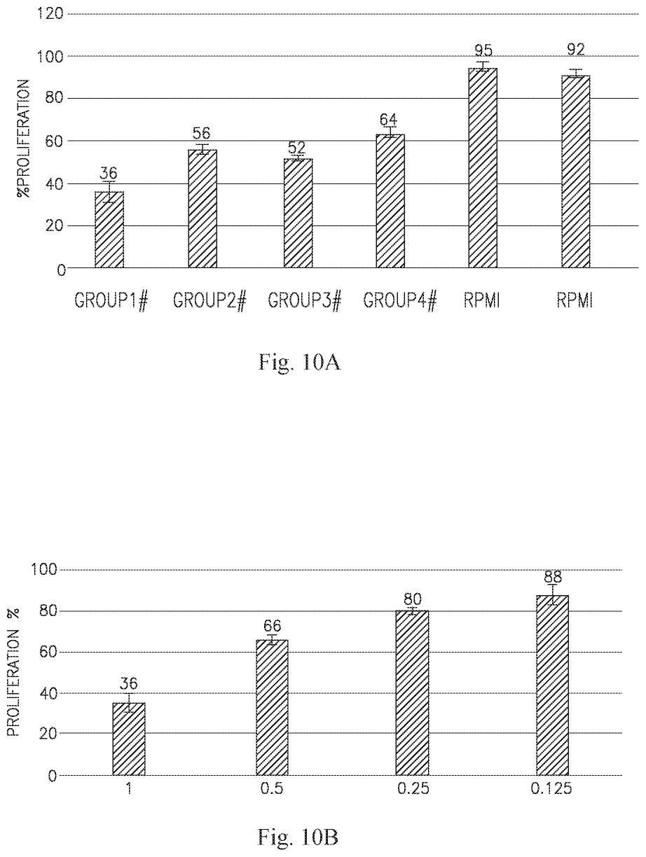

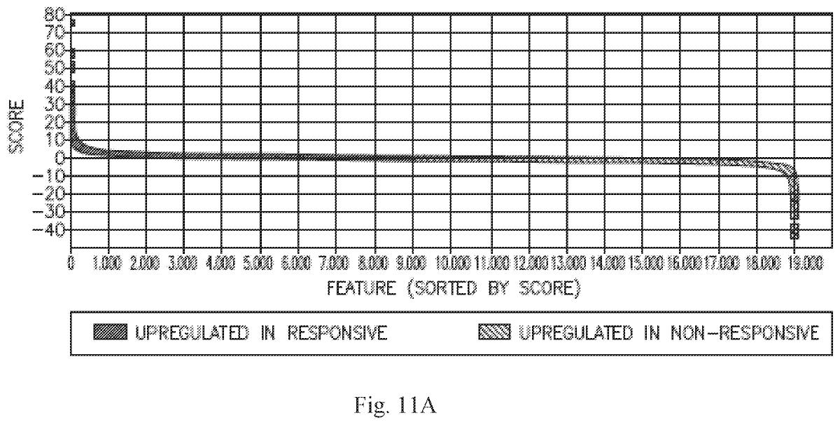

Several cancer types exhibited inhibition (at least a 40% reduction in proliferation) by at least the highest concentration of Group 1, namely renal cell carcinoma (2/4 cell lines tested; FIG. 6), melanoma (1/4 lines tested), hepatocellular carcinoma (2/5 lines; FIG. 7), colorectal carcinoma (2/10 lines), breast carcinoma (2/6 lines, namely MDA-MB-231 and HCC-1395; FIGS. 8A-D), lung adenocarcinoma (1/1 lines; FIG. 9), large cell lung carcinoma (1/1 lines), and rhabdomyosarcoma (1/1 lines; FIG. 10).

### Example 9

### Differentially Expressed Gene Analysis Between Responsive Cell Lines and Other Cell Lines

To identify marker genes that are differentially expressed between the responsive cancer cell lines and the other cell lines from the previous Example, the cell lines were grouped by organ. Therefore, the cancer cell lines chosen for marker gene selection came from five organs: breast, large intestine, kidney, liver and lung. The two responsive cell lines RD (rhabdomyosarcoma) and CHL-1 (melanoma) were excluded from the investigation, because there was insufficient data to ensure two cell lines for each of the two classes for cancers originating from these organs.

The two classes were assigned for each organ as follows (Table 15):
Class 0: responsive cell lines defined as having a percent of control proliferation (POC) ≤60% with undiluted CM from the ASC-TNFα/INFγ treatment.
Class 1: cell lines having a POC≥79% with undiluted CM from the ASC-TNFα/INFγ treatment.

Marginally responsive cell lines defined as having 60%<POC<79% were excluded from both classes.

TABLE 15
The cell line matrix for ComparativeMarkerSelection input

| Organ | Class 0 Cell Lines | Class 1 Cell Lines |
|---|---|---|
| Breast | HCC1395 | BT474 |
| | MDAMB231 | MCF7 |
| | | T47D |
| Large Intestine | HT29 | COLO320 |
| | SW48 | DLD1 |
| | | HCT15 |
| | | SW480 |
| | | SW620 |
| | | HCT116 |
| Liver | HEPG2 | C3A |
| | SNU449 | HUH7 |
| Lung | NCIH1792 | A549 |
| | NCIH460 | NCIH23 |
| Kidney | 769P | ACHN |
| | 786O | A498 |

Next, gene expression data was obtained from the Cancer Cell Line Encyclopedia (CCLE; Barretina, J., et al). The CCLE provides public access to genomic data, analysis and visualization for over 1000 cell lines. mRNA expression array data for the cancer cell lines that were used in the cell proliferation assay were downloaded for identifying marker genes. Prior to downloading the data sets, the raw Affymetrix CEL files from the original Affymetrix U133+2 arrays were converted to a single value for each probe set using Robust Multi-array Average (RMA) and normalized using quantile normalization. A redefined custom CDF file from the package HGU133Plus2_Hs_ENTREZG_15.0.0 from Brainarray was used for the summarization.

In order to identify and select marker genes, the ComparativeMarkerSelection module in GenePattern (Reich et al) was employed.

Genes were scored by calculating the value of the two-sided t-test for each profiled gene. Marker genes were selected if the test statistic was >5 or <−5. Positive values indicate upregulated genes in the responsive cell lines, while negative values indicate downregulated genes in the responsive cell lines. Table 16 shows the numbers of marker genes with scores >5 and <−5.

TABLE 16
Numbers of marker genes with scores >5 and <−5

| | | | Large | | |
|---|---|---|---|---|---|
| | Breast | Kidney | Intestine | Liver | Lung |
| Up | 412 | 494 | 91 | 190 | 297 |
| Down | 382 | 318 | 112 | 151 | 452 |
| Total | 794 | 812 | 203 | 341 | 749 |

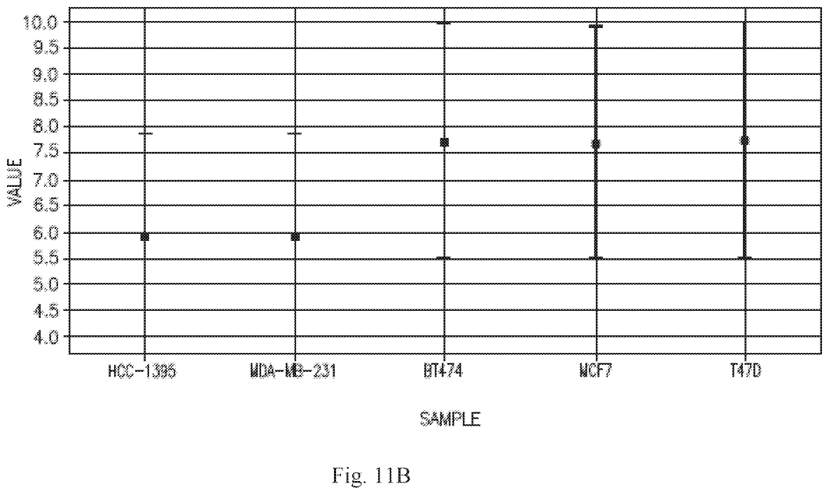

As an example, FIG. 11A depicts a graphical representation of the scores for each profiled gene for the breast cancer cell lines analysis. The upregulated genes in the responsive cell lines are shown on the left side of the graph, while the downregulated genes in the responsive cell lines (upregulated in the other cell lines) are shown on the right side. FIG. 11B is a centroid plot showing the mean expression value for the five breast cancer cell lines for all of the genes downregulated (scores <−5) in the responsive breast cell lines. The two responsive breast cancer cell lines (HCC-1395 and MDA-MB-231) are shown on the left, and the other three breast cancer cell lines (BT474, MCF7 and T47D) are shown on the right.

### Example 10

### Identification of Pathways Significantly Perturbed in Responsive Cell Lines

To determine relevant biological pathways that are perturbed between Class 0 and Class 1 cell lines within each organ, the most statistically significantly upregulated and downregulated genes within each organ were used to probe the Reactome Pathway Database V53 (Croft et al). Table 17 shows the number of upregulated and downregulated genes from each of the five organs that are found in at least one Reactome pathway.

TABLE 17
Numbers of up- and downregulated genes found in Reactome pathways
DE Genes Responsive Cell Lines vs Other (Score >5 and <−5)

| | | | Large | | |
|---|---|---|---|---|---|
| | Breast | Kidney | Intestine | Liver | Lung |
| Up | 412 | 494 | 91 | 190 | 297 |
| Down | 382 | 318 | 112 | 151 | 452 |
| Total | 794 | 812 | 203 | 341 | 749 |
| Up-Reactome | 191 | 213 | 26 | 62 | 127 |
| Down-Reactome | 120 | 74 | 58 | 63 | 93 |
| Total | 311 | 287 | 84 | 125 | 220 |

The data was searched for significant pathways that were common among the organs. The following 5 pathways appeared in the list of the top 200 Reactome pathways in 4/5 output lists:
1. RIG-I/MDA5 mediated induction of IFN-alpha/beta pathways (R-HSA-168928)
2. Interferon Signaling (R-HSA-913531)
3. Cytokine Signaling in Immune system (R-HSA-1280215)
4. Cellular Senescence (R-HSA-2559583)
5. Deactivation of the beta-catenin trans-activating complex (R-HSA-3769402)

Next, the most statistically significant upregulated and downregulated genes were pooled from each of the five marker gene sets and used to probe the Reactome Pathway Database. The database was probed three times:
1. All upregulated genes
2. All downregulated genes
3. All upregulated and downregulated genes

The statistical cut-off for defining a biological pathway as being statistically significant was an entities false discovery rate (FDR)≤0.05. Tables 18-20 reveal the most statistically relevant biological pathways that are perturbed between Class 0 and Class 1 across the five organs due to upregulated genes, downregulated genes, and both, respectively. Note that the bold-faced pathways in Table 19 survive the selection process when mutated genes are added to the analysis, as can be seen below in Table 22.

TABLE 18
Most Statistically Significant Pathways
Perturbed Due to Upregulated Genes

| Pathway name | Entities pValue | Entities FDR |
|---|---|---|
| Factors involved in megakaryocyte | 1.08E−05 | 1.59E−02 |
| development and platelet production | | |
| Cellular Senescence | 2.06E−05 | 1.59E−02 |
| Mitochondrial biogenesis | 4.83E−05 | 2.30E−02 |
| Hemostasis | 5.95E−05 | 2.30E−02 |
| Signaling by NOTCH | 1.03E−04 | 3.18E−02 |
| Organelle biogenesis and maintenance | 1.84E−04 | 4.73E−02 |

TABLE 19
Most Statistically Significant Pathways
Perturbed Due to Downragulated Genes

| Pathway name | Entities pValue | Entities FDR |
|---|---|---|
| Interferon alpha/beta signaling | 6.32E−09 | 8.18E−06 |
| Cytokine Signaling in Immune | 3.78E−08 | 2.44E−05 |
| system | | |
| Interferon Signaling | 8.83E−08 | 3.81E−05 |
| Ion channel transport | 4.33E−05 | 1.28E−02 |
| Interferon gamma signaling | 4.95E−05 | 1.28E−02 |
| RIG-I/MDA5 mediated induction of | 8.29E−05 | 1.78E−02 |
| IFN-alpha/beta pathways | | |
| Activation of gene expression by | 1.23E−04 | 2.27E−02 |
| SREBF (SREBP) | | |
| PPARA activates gene expression | 2.53E−04 | 3.99E−02 |
| Endosomal/Vacuolar pathway | 3.15E−04 | 3.99E−02 |
| Regulation of lipid metabolism by | 3.16E−04 | 3.99E−02 |
| PPARalpha | | |
| TRAF6 mediated IRF7 activation | 3.41E−04 | 3.99E−02 |

TABLE 20
Most Statistically Significant Pathways Perturbed
Due to Upregulated and Downregulated Genes

| Pathway name | Entities pValue | Entities FDR |
|---|---|---|
| PPARA activates gene expression | 1.47E−05 | 1.82E−02 |
| Regulation of lipid metabolism | 2.16E−05 | 1.82E−02 |
| by PPARalpha | | |

As can be seen from these results, the biological pathways that show the highest statistical significance are revealed when the database is probed by the pooled set of downregulated genes set from Class 0.

### Example 11

### Mutation Analysis of Exomes Between Responsive Cell Lines and Other Cell Lines

To help determine the pathways of the greatest biological significance, in addition to the described statistical significance, somatic mutations were analyzed via full exome sequencing from the COSMIC Cancer Cell Lines Project (Forbes et al). Addition, deletion, substitution, frameshift and splice site mutations were included in the count, whereas CDS silent mutations were excluded. Data from Hep-G2, AGS, DLD1, LS-174T and SW480 did not appear in the database. Besides these cell lines, a total of >10,300 mutations were counted in the 11 responsive cell lines and >53,000 mutations in the other 43 cell lines.

The Reactome database was probed with:
all genes mutated exclusively in the 11 responsive cell lines
all genes mutated exclusively in the other 43 cell lines (the marginal and non-responsive lines).
the pooled list of all genes mutated exclusively in the responsive cell lines; and all genes downregulated in the responsive cell lines (as described for Table 17 above).

The final query was the most informative, so its results are described hereinbelow.

The numbers of genes exclusively mutated in the responsive cell lines are shown in the Table 21:

TABLE 21

| | 769-P | 786-O | SW48 | HT-29 | HCC1395 | MDA-MB-231 |
|---|---|---|---|---|---|---|
| | Kidney | Kidney | Colon | Colon | Breast | Breast |
| Mutated (only | 221 | 222 | 1904 | 345 | 276 | 375 |
| in Responsive) | | | | | | |
| Mutated | 95 | 103 | 871 | 148 | 121 | 151 |
| (Reactome) | | | | | | |

| | NCI-H1792 | NCI-H460 | SNU-449 | Hep G2 | CHL-1 | RD |
|---|---|---|---|---|---|---|
| | Lung | Lung | Liver | Liver | Skin | Muscle |
| Mutated (only | 255 | 303 | 343 | — | 764 | 233 |
| in Responsive) | | | | | | |
| Mutated | 126 | 144 | 154 | — | 372 | 104 |
| (Reactome) | | | | | | |

The results of the final query are shown in Table 22. The statistical cut-off was an entities FDR≤0.05.

TABLE 22

| | | | Reac- | Reac- |
|---|---|---|---|---|
| | Entities | Entities | tions | tions |
| Pathway name | pValue | FDR | found | total |
| Endosomal/Vacuolar pathway | 1.11E−16 | 9.78E−14 | 4 | 4 |
| Interferon alpha/beta signaling | 1.11E−16 | 9.78E−14 | 14 | 19 |
| Antigen Presentation: Folding, | 3.77E−15 | 2.22E−12 | 13 | 14 |
| assembly and peptide loading | | | | |
| of class I MHC | | | | |
| Interferon gamma signaling | 1.34E−12 | 5.89E−10 | 11 | 15 |
| Interferon Signaling | 2.77E−12 | 9.74E−10 | 41 | 50 |
| ER-Phagosome pathway | 8.61E−12 | 2.52E−09 | 3 | 5 |
| Antigen processing-Cross | 3.82E−10 | 9.59E−08 | 8 | 17 |
| presentation | | | | |
| Class I MHC mediated antigen | 5.35E−08 | 1.18E−05 | 28 | 39 |
| processing & presentation | | | | |
| Cytokine Signaling in Immune | 7.45E−07 | 1.45E−04 | 200 | 285 |
| system | | | | |

This analysis showed that the responsive cancer cell lines are those cell lines that have a downregulation or a dysregulation in two significant pathways: MHC Class I antigen processing and presentation (which includes the endosomal/vacuolar, antigen presentation: folding, assembly and peptide loading of class I MHC, ER-phagosome, and antigen processing-cross presentation pathways) and cytokine signaling (which includes the interferon alpha/beta signaling, interferon gamma signaling, and interferon signaling pathways). These pathways overlap significantly with the pathways found statistically significantly in the previous analysis (Table 19), thus validating the statistical analyses presented herein and the importance of these particular pathways.

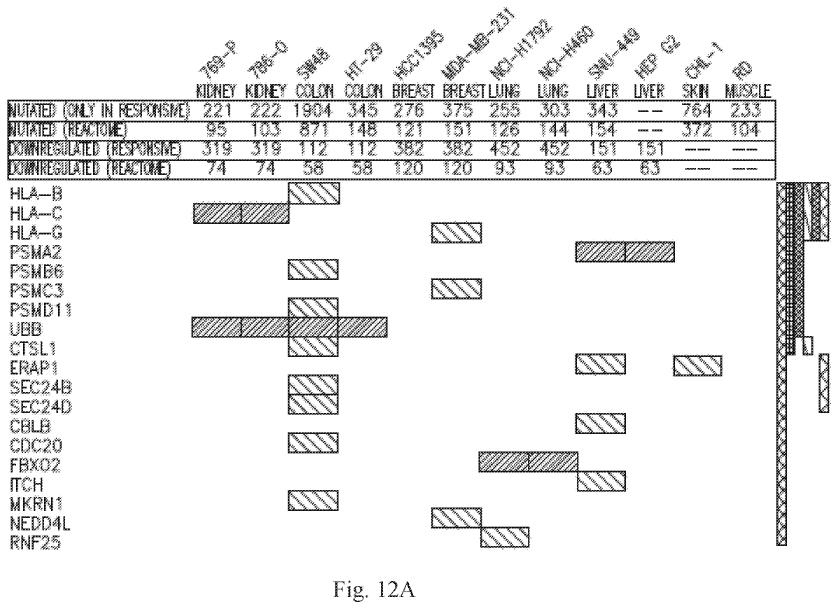

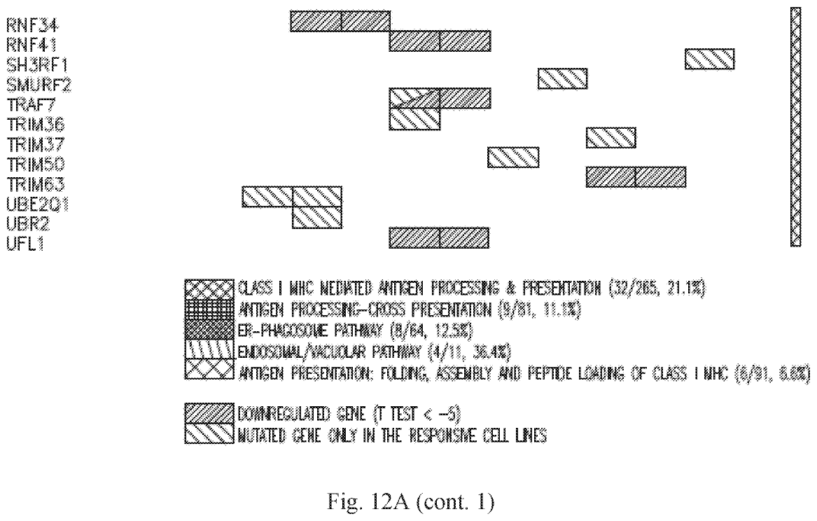

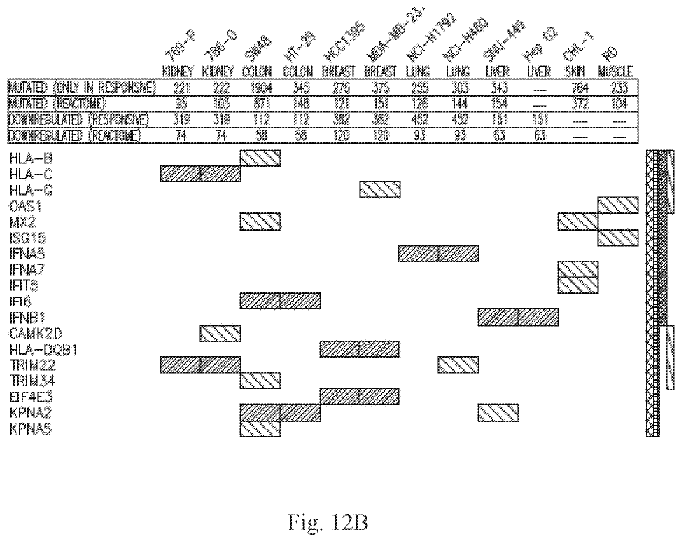

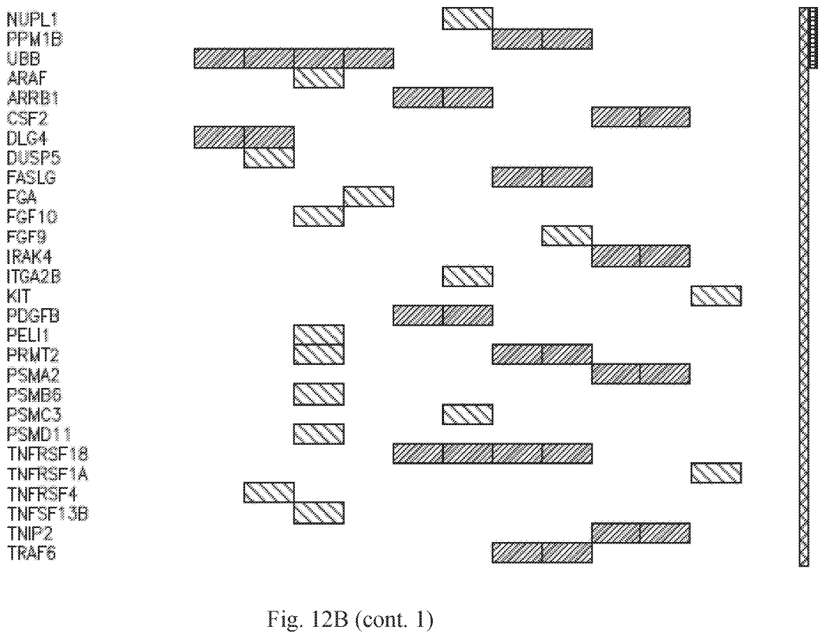

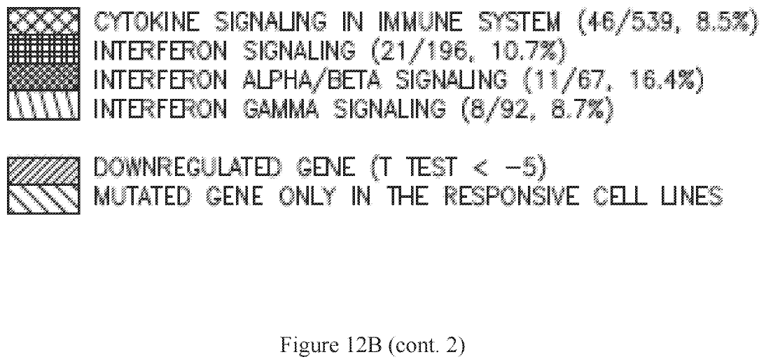

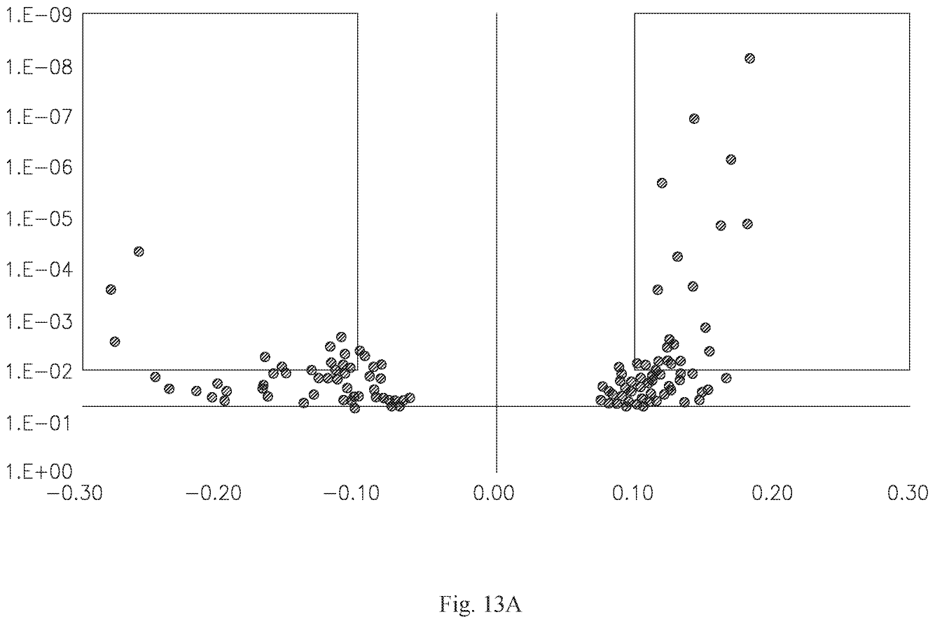

FIGS. 12A-B summarize the genes in these pathways that are downregulated and/or exclusively mutated in each of the responsive cell lines.

In conclusion, the above data show that cancer cell lines with downregulated or dysregulated MHC Class I antigen processing and presentation pathways and/or downregulated or dysregulated cytokine signaling pathways are sensitive to treatment with ASC.

### Example 12

### Further Characterization of Responsive and Non-Responsive Breast Cancer Cell Lines

Methods

Similarly to Example 11, somatic mutations were analyzed via full exome sequencing from the COSMIC Cancer Cell Lines Project (Forbes et al). Addition, deletion, substitution, frameshift and splice site mutations were included in the count, whereas CDS silent mutations were excluded. Data from Hep-G2, AGS, DLD1, LS-174T and SW480 did not appear in the database. Besides these cell lines, a total of >10,300 mutations were counted in the 11 responsive cell lines and >53,000 mutations in the other 43 cell lines. An averaged POC together with its standard deviation was calculated by taking the mean of the POCs in each of the cell lines in which each mutated gene was present. Likewise, an averaged POC together with its standard deviation was calculated by taking the mean of the POCs in each of the cell lines in which each mutated gene was not present. P values were calculated for each mutated gene, and these p values were plotted against the log effect of the response. Log effect was calculated by subtracting the logarithm of the averaged POC in the cell lines without the mutation from the logarithm of the averaged POC in the cell lines with the mutation.

Results

Additional analyses were performed to identify somatic mutations present in cancer cell lines that correlated with responsiveness to ASC treatment or lack of responsiveness, referred to herein as “ASC treatment informative mutations”. When limiting the results to genes that were mutated in at least four cell lines, mutations in 295 genes were found that positively correlated with responsiveness (such mutations and genes are referred to herein as “ASC sensitivity mutations”) and “ASC sensitivity genes”, respectively), while mutations in 316 gene negatively correlated with responsiveness (such mutations and genes are referred to herein as “ASC resistance mutations” and “ASC resistance genes”, respectively), a number of which exhibited a log effect absolute value of >0.1 and a p-value of <0.01 (FIG. 13A). FIGS. 13B-C show the specific mutations found in the genes that were negatively and positively correlated with responsiveness, respectively. Table 23 shows the top 16 mutations positively correlated with responsiveness, ranked by their p-value, namely TAF1, ZNF248, DPY19L4, SCN3A, DCHS1, PDGFRA, LGSN, EPHB4, SEMA3E, EXTL3, SFMBT1, DUOX2, CCDC137, PCDH12, TLR1, and GPR124. The top 3 mutations (TAF1, ZNF248, and DPY19L4) were mutated in none or only 1 of the non-responsive cell lines. Table 24 shows the top 22 mutations (ZNF708, PRG4, CTU2, GOLGA8A, PTCH2, NSD1, QRICH2, SPAG5, C6orf165, LIMK2, EIF4B, LATS1, SCN8A, VPS8, KIAA1161, AFF3, KIAA1715, SLC6A17, SF1, KIAA0494, ZNF592, and BAZ2B) negatively correlated with responsiveness, ranked by their p-value. None of these 22 mutations appeared in any of the non-responsive cell lines. ASC sensitivity genes and ASC sensitivity genes are collectively referred to herein as “ASC-susceptibility genes”.

TABLE 23

| | | | | | Mutations |
|---|---|---|---|---|---|
| | | Cell Lines | | | that |
| Mutated | Mutated | with | Log | | Enhance |
| (Responsive) | (Other) | Mutation | Effect | P Value | Treatment |
| 4: 36% | 0: 0% | 4 | −0.28 | 2.3E−04 | TAF1 |
| 3: 27% | 1: 2% | 4 | −0.28 | 2.4E−03 | ZNF248 |
| 5: 45% | 0: 0% | 5 | −0.26 | 4.2E−05 | DPY19L4 |
| 4: 36% | 5: 12% | 9 | −0.17 | 5.0E−03 | SCN3A |
| 3: 27% | 4: 9% | 7 | −0.16 | 8.1E−03 | DCHS1 |
| 4: 36% | 3: 7% | 7 | −0.15 | 8.7E−03 | PDGFRA |
| 3: 27% | 3: 7% | 6 | −0.13 | 9.0E−03 | LGSN |
| 3: 27% | 2: 5% | 5 | −0.12 | 3.1E−03 | EPHB4 |
| 3: 27% | 2: 5% | 5 | −0.12 | 6.2E−03 | SEMA3E |
| 2: 18% | 2: 5% | 4 | −0.12 | 9.2E−03 | EXTL3 |
| 2: 18% | 2: 5% | 4 | −0.11 | 1.9E−03 | SFMBT1 |
| 2: 18% | 2: 5% | 4 | −0.11 | 2.1E−03 | DUOX2 |
| 2: 18% | 2: 5% | 4 | −0.11 | 7.3E−03 | CCDC137 |
| 2: 18% | 3: 7% | 5 | −0.11 | 8.3E−03 | PCDH12 |
| 2: 18% | 2: 5% | 4 | −0.11 | 4.6E−03 | TLR1 |
| 2: 18% | 2: 5% | 4 | −0.11 | 8.6E−03 | GPR124 |

TABLE 24

| | | | | | Mutations |
|---|---|---|---|---|---|
| | | Cell Lines | | | that |
| Mutated | Mutated | with | Log | | Exacerbate |
| (Responsive) | (Other) | Mutation | Effect | P Value | Treatment |
| 0: 0% | 4: 9% | 4 | 0.18 | 6.6E−09 | ZNF708 |
| 0: 0% | 4: 9% | 4 | 0.18 | 1.2E−05 | PRG4 |
| 0: 0% | 4: 9% | 4 | 0.17 | 6.6E−07 | CTU2 |
| 0: 0% | 5: 12% | 5 | 0.16 | 1.3E−05 | GOLGA8A |
| 0: 0% | 4: 9% | 4 | 0.15 | 3.9E−03 | PTCH2 |
| 0: 0% | 4: 9% | 4 | 0.15 | 1.3E−03 | NSD1 |
| 0: 0% | 5: 12% | 5 | 0.14 | 1.0E−07 | QRICH2 |
| 0: 0% | 4: 9% | 4 | 0.14 | 2.0E−04 | SPAG5 |
| 0: 0% | 5: 12% | 5 | 0.13 | 6.2E−03 | C6orf165 |
| 0: 0% | 4: 9% | 4 | 0.13 | 5.2E−05 | LIMK2 |
| 0: 0% | 4: 9% | 4 | 0.13 | 2.7E−03 | EIF4B |
| 0: 0% | 5: 12% | 5 | 0.13 | 2.6E−03 | LATS1 |
| 0: 0% | 5: 12% | 5 | 0.13 | 6.7E−03 | SCN8A |
| 0: 0% | 4: 9% | 4 | 0.12 | 2.3E−03 | VPS8 |
| 0: 0% | 4: 9% | 4 | 0.12 | 3.2E−03 | KIAA1161 |
| 0: 0% | 4: 9% | 4 | 0.12 | 6.2E−03 | AFF3 |
| 0: 0% | 4: 9% | 4 | 0.12 | 1.9E−06 | KIAA1715 |
| 0: 0% | 4: 9% | 4 | 0.12 | 9.6E−03 | SLC6A17 |
| 0: 0% | 4: 9% | 4 | 0.12 | 6.1E−03 | SF1 |
| 0: 0% | 4: 9% | 4 | 0 12 | 2.4E−04 | KIAA0494 |
| 0: 0% | 4: 9% | 4 | 0.11 | 7.1E−03 | ZNF592 |
| 0: 0% | 9: 21% | 9 | 0.10 | 6.8E−03 | BAZ2B |

These results clearly identify mutations associated with a therapeutic response, or lack thereof, of tumors to treatment with ASC.

### Example 13

### Further Characterization of Responsive and Non-Responsive Breast Cancer Cell Lines

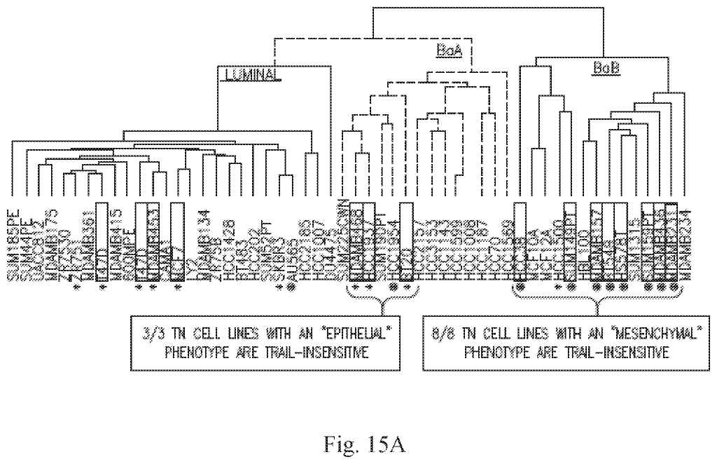

Next, the phenotypes of responsive and non-responsive breast cancer cell lines were determined, based on expression of 305 classifier genes useful for characterizing breast cancer lines as Luminal, Basal A, or Basal B by hierarchical clustering (Neve et al). The data from the paper was downloaded and reproduced (FIG. 14) using the GenePattern software tool (Reich et al). Pearson Correlation Clustering was used for distance measurements for both columns and rows, and the hierarchical clustering method was pairwise average-linkage.

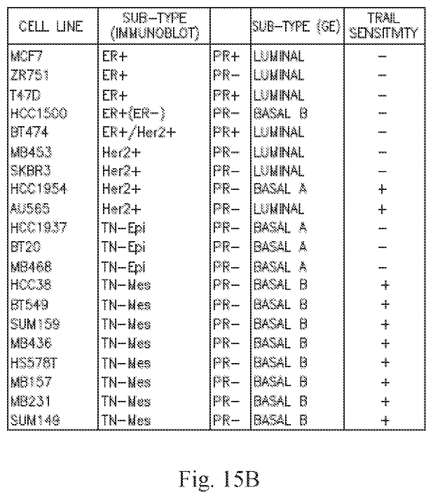

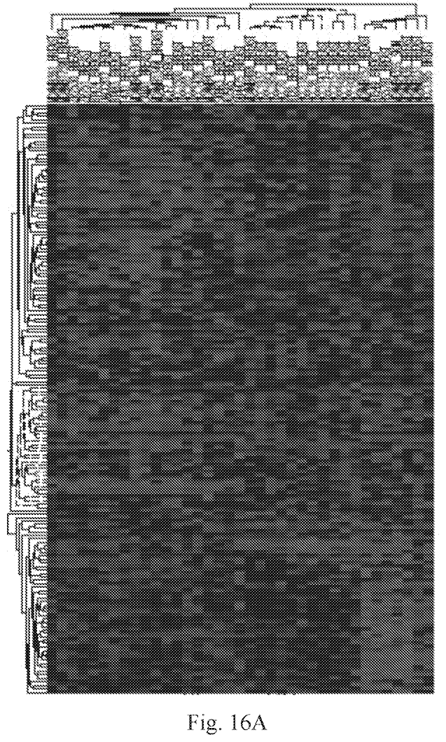

FIG. 15A depicts the top of FIG. 14, showing which breast cancer cell lines are characterized, which include 5/6 cell lines that were tested herein for ASC sensitivity; these are highlighted in blue.

FIG. 15A also incorporates data from Rahman et al, which tested 20 breast cancer cell lines, including 11 triple negative (TN) lines, for TRAIL sensitivity; these are marked by black asterisks (TRAIL-insensitive) and red asterisks (TRAIL-sensitive). Most of the TN cell lines that are TRAIL-sensitive fall in the Basal B cluster, although there are 2 that fall outside it. Nevertheless, of the 8 TRAIL-sensitive TN cell lines that are Basal B, they all have the “mesenchymal phenotype” and all of the 3 TRAIL-insensitive that are Basal A have the “epithelial phenotype.”

The mesenchymal phenotype is defined as having high levels of Vimentin, high levels of caveolins, and low levels of E-cadherin. The epithelial phenotype is defined as having high levels of E-cadherin, abundant keratins, and low levels of Vimentin.

FIG. 15B depicts the data from tested breast cancer cell lines from FIG. 15A in tabular form, and also includes information on clinical sub-type, namely whether or not estrogen receptor (ER) or progesterone receptor (PR) is present, and whether or not Her2/neu is amplified.

In conclusion, these data show that TN breast tumors exhibit sensitivity to treatment with ASC. This may be particularly true of TRAIL-sensitive TN breast tumors with a mesenchymal phenotype.

HCC1395 is the only breast cancer line that was tested herein for ASC sensitivity and was not analyzed in the aforementioned hierarchical clustering analysis by Neve et al. This cell line was used to verify the hypothesis that sensitivity of breast tumors to ASC parallels the TN phenotype and TRAIL sensitivity. Since HCC1395 was sensitive to ASC treatment, the working hypothesis would predict that the triple negative cell line HCC1395 is TRAIL-sensitive and falls into the Basal B cluster. This analysis required another dataset. The Cancer Cell Line Encyclopedia (CCLE; Barretina et al) was probed for breast cancer cell lines that were also in the hierarchical clustering analysis by Neve et al. Affymetrix gene expression data from 37 breast cancer cell lines was downloaded and processed as described hereinabove.

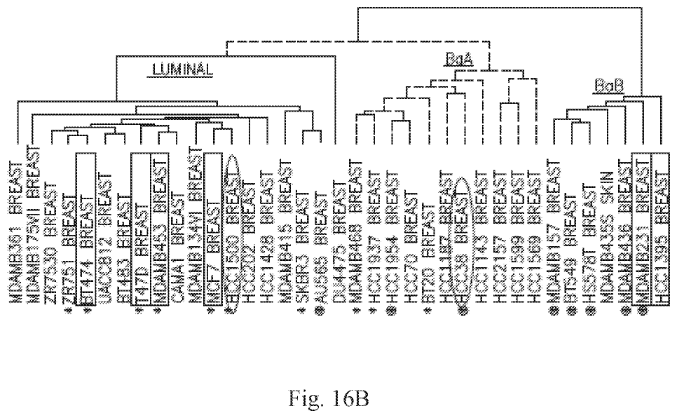

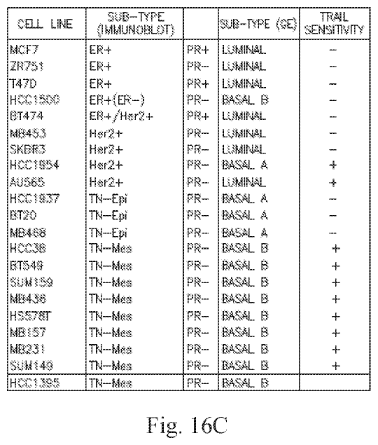

“Affy probes to gene” matches were used to identify genes in the Neve et al dataset, which were in turn used to select relevant gene expression data out of the 37 lines from the CCLE. This process yielded 169 probe sets to perform the hierarchical clustering, which yielded a similar clustering (FIG. 16A) to Neve et al. It is clear from this analysis that HCC-1395 clustered together with MDA-MB-231 in the TN/Basal B group, thus verifying the hypothesis that sensitivity of breast tumors to ASC parallels TN phenotype and TRAIL sensitivity.

FIG. 16B shows the top of FIG. 16A. Only 2 cell lines (circled) clustered differently than in the previous analysis. A virtually identical hierarchical clustering was obtained whether 18,000 probe sets (the number of probes in the gene expression data from the set of 37 CCLE cell lines) or 169 probe sets were used, thus verifying the clustering scheme. This also verifies that TN cell lines in the Basal B cluster are TRAIL sensitive (FIG. 16C).

### Example 14

### Genes Responsible for Clustering of Breast Tumor Lines are Involved in Antigen Presentation and IFN Signaling

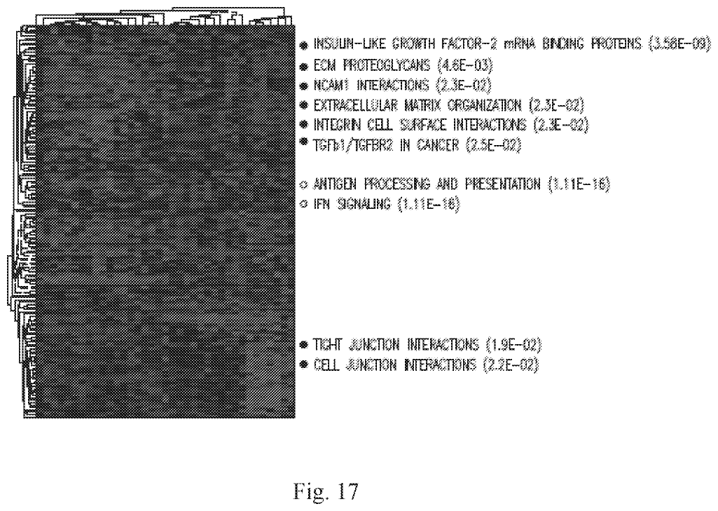

The genes identified the aforementioned hierarchical clustering analysis by Neve et al genes is responsible for clustering into Luminal, Basal A and Basal B were entered into the Reactome Pathway Database (each section individually) to identify pathways in which the classifier genes participate (FIG. 17). The middle rows section of classifier genes included HLAs and a few other antigen processing/presentation genes as well as IFN signaling pathway genes, thus validating the aforementioned analyses and verifying that the previously-identified pathways apply to breast cancer cell lines.

### Example 15

### Trail-Sensitive Cancer Cell Lines are Sensitive to ASC Treatment

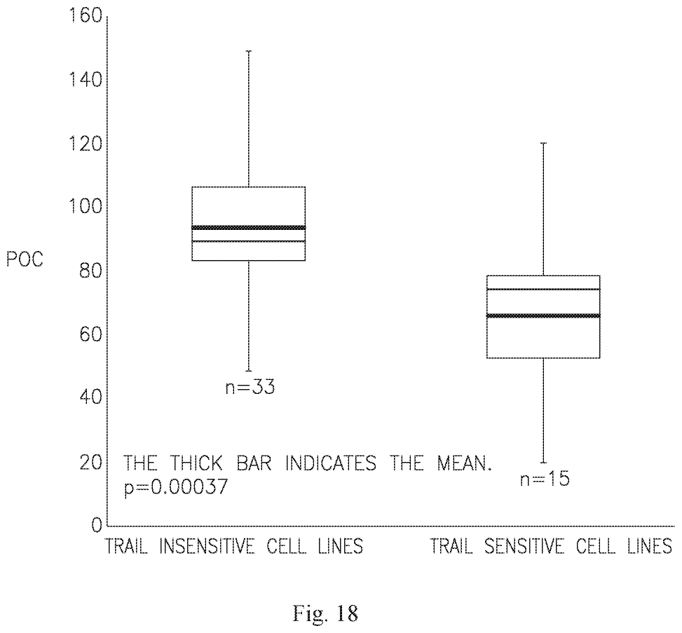

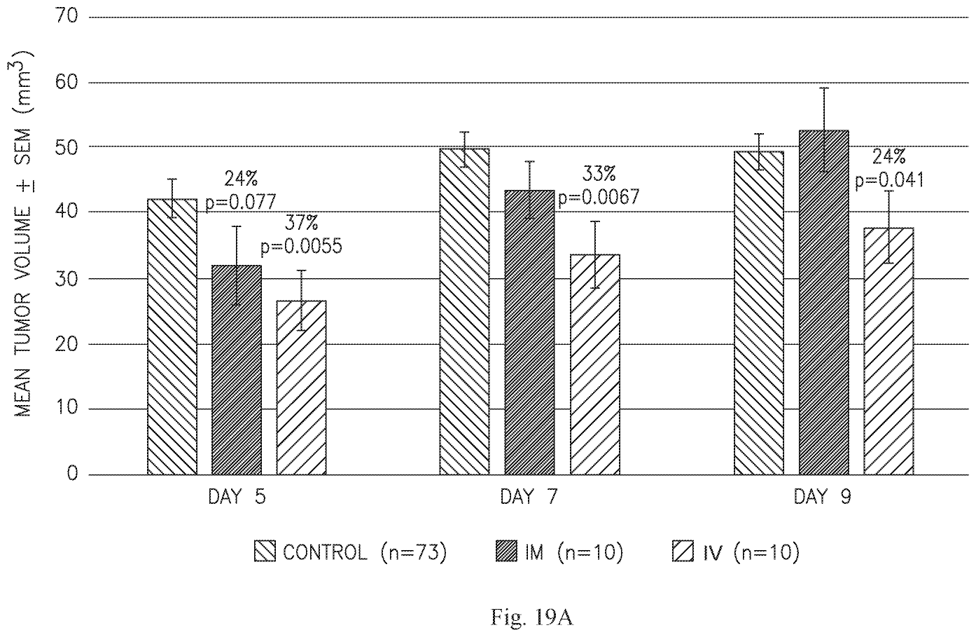

The scientific literature was combed for indications of the TRAIL sensitivity of the 59 cell lines that were tested herein for sensitivity to ASC treatment. A clear indication was found for 48 of the 59 cell lines. TRAIL sensitivity of these 48 lines was plotted vs. ASC sensitivity, and the two parameters were found to correlate with one another (FIG. 18). The mean proliferation values observed in the TRAIL-sensitive and TRAIL-insensitive lines were 66% and 94%, respectively. These numbers were close to the values assigned for a positive response and a non-response to ASC, respectively (60% and 100%, respectively). The p-value for the difference between the groups was 0.00037.

This analysis shows that there is a strong correlation between TRAIL-sensitivity and ASC-sensitivity in the tested cancer cell lines.

### Example 16

### In Vivo Testing of Asc in a Tumor Implantation Model

Methods

93 athymic Foxn1^{nu} nude were injected (all groups) subcutaneously in the right flank with 3×10⁶ MDA-MB-231 adenocarcinoma cells in 0.2 ml PBS—this was considered day 0.

The experimental groups are shown in Table 25:

TABLE 25

| | | | Time of |
|---|---|---|---|
| Group num/name | Intervention | Route | intervention |
| 1/Untreated control | None | — | — |
| 2/IM control | PlasmaLyte A | IM | Day 9 only |
| 3/IV control | PlasmaLyte A | IV | Day 9 only |
| 4/IM Late Treatment | 1 × 10⁶ TNF/IFN | IM | Day 9 only |
| | stimulated ASC | | |
| 5/IV Late Treatment | 1 × 10⁶ TNF/IFN | IV | Day 9 only |
| | stimulated ASC | | |
| 6/IM early/late treat. | 1 × 10⁶ TNF/IFN | IM | Days 1 and 9 |
| | stimulated ASC | | |
| 7/IV early/late treat. | 1 × 10⁶ TNF/IFN | IV | Days 1 and 9 |
| | stimulated ASC | | |

TNF-α+IFN-γ-stimulated ASC were suspended in a volume of 50 mcl (microliters) or 250 mcl for intramuscular (IM) or intravenous (IV) administration, respectively. On day 1, ASC were administered IM or IV, to 10 mice each in Groups 6 and 7, respectively. On day 9, 23 mice with tumor sizes outside the range of 36-88 mm³ were removed from the 73 untreated mice (previously referred to as the control group), leaving 50 untreated mice. The 50 animals were assigned randomly to Groups 1, 2, 3, 4 and 5. Randomization was performed according to the size of the tumor such that each group ended up with mice having tumors approximately the same average size. Thus there were ultimately 10 mice in each group. On the same day, mice were administered either no treatment, mock IM or IV injection (Groups 1, 2, and 3, respectively); or ASC administered IM (Groups 4 and 6, receiving a first or additional treatment, respectively) or IV (Groups 5 and 7, receiving a first or additional treatment, respectively), as indicated in Table 25.

Tumor volume was measured using electronic calipers.

Results

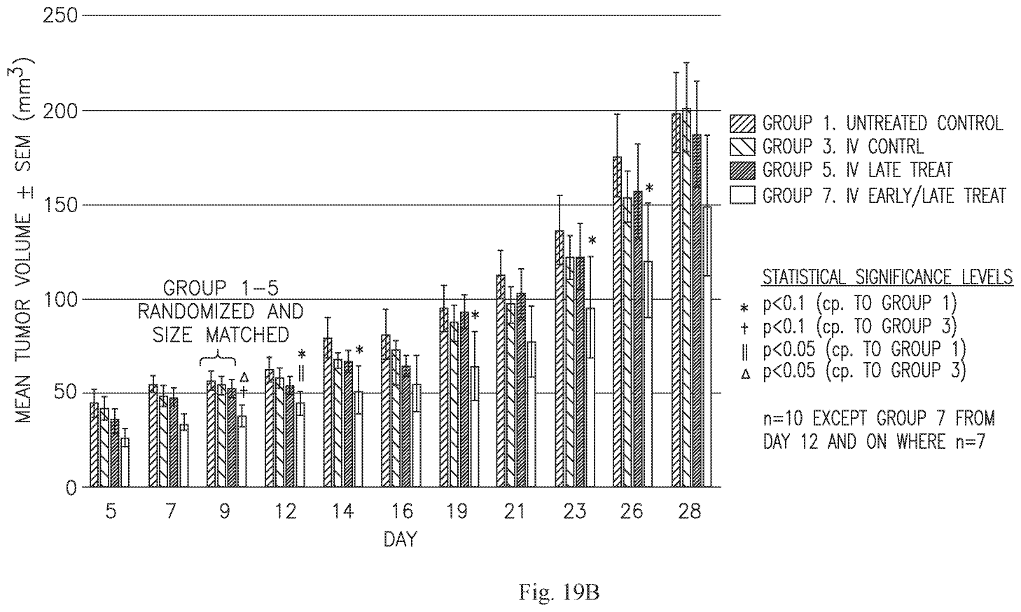

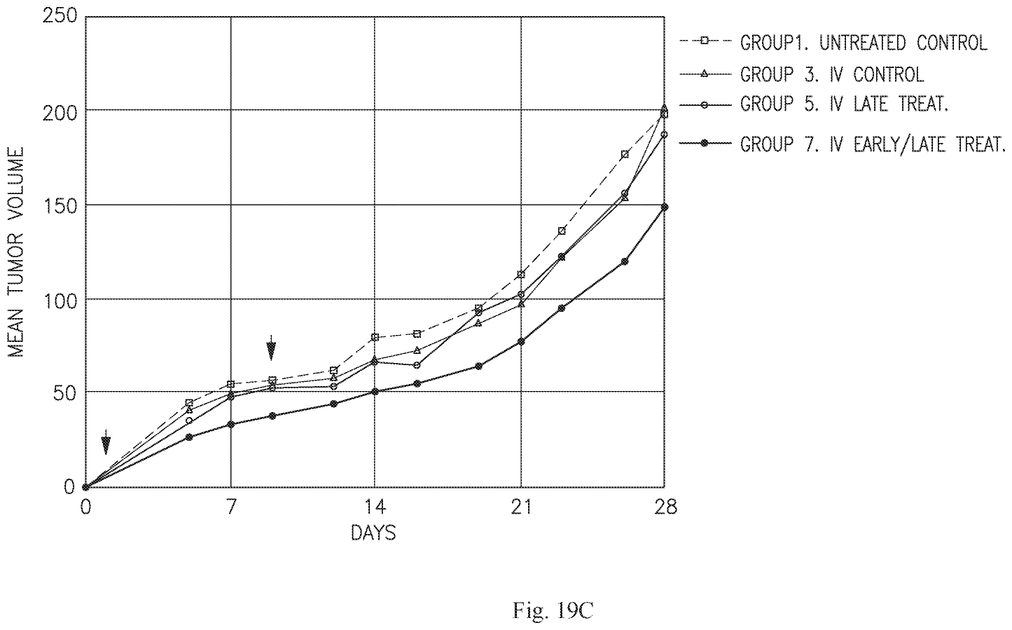

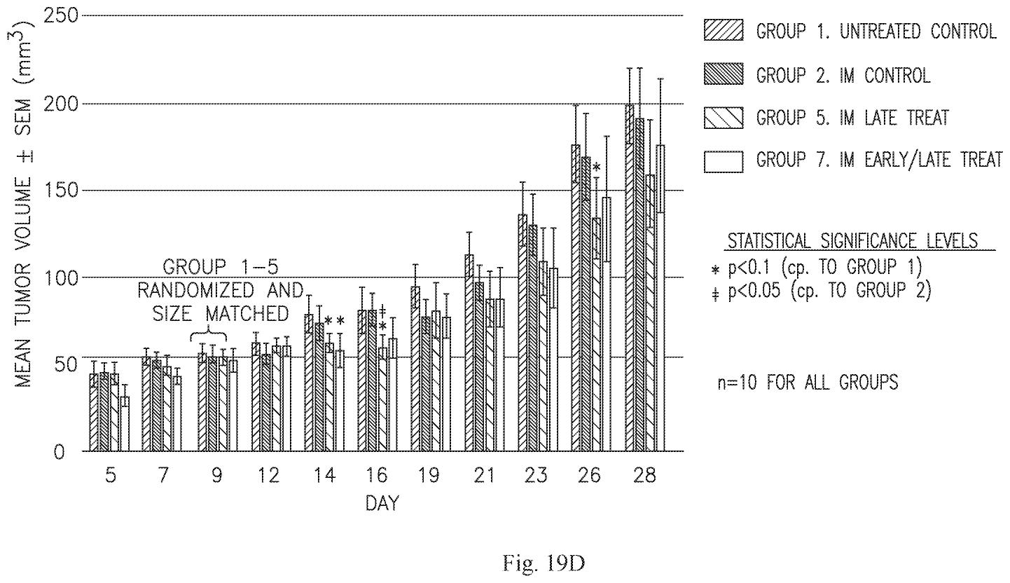

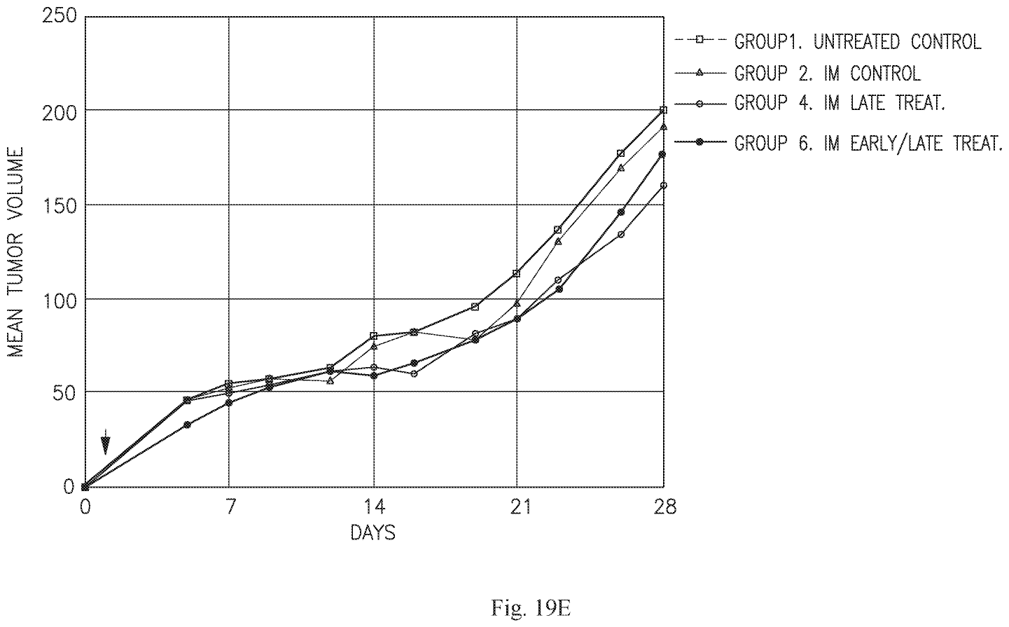

Anti-tumor effects of ASC were tested in an in vivo tumor implantation model. Mice receiving ASC administration on day 1 exhibited reduced tumor size. The inhibition was statistically significant when IV was compared to controls at each time point, namely at days 5, 7, and 9 (p=0.0055, 0.0067, and 0.041 by one-tailed t-test, respectively) (FIG. 19A). Trends of efficacy were seen in both IV-injected (FIGS. 19B-C) and IM-injected (FIGS. 19D-E) mice. The inhibitory effect was strongly seen when observing the fold change in tumor volume from days 12-16 (Tables 26-27); days 9-28 (Table 28); and days 9-16 (Table 29).

TABLE 26
Fold Change in Tumor Volume from Day 12-16

| | Untreated | | IM ASC | IM ASC |
|---|---|---|---|---|
| | Controls | IM Control | (Late) | (Early/Late) |
| | (Group 1) | (Group 2) | (Group 4) | (Group 6) |
| Mean | 1.33 | 1.52 | 1.01 | 1.06 |
| Standard | 0.53 | 0.47 | 0.39 | 0.35 |
| Deviation (SD) | | | | |
| Minimum | 0.32 | 1.00 | 0.50 | 0.52 |
| 1st Quartile | 1.03 | 1.07 | 0.84 | 0.87 |
| 2nd Quartile | 1.34 | 1.48 | 1.00 | 1.02 |
| (Median) | | | | |
| 3rd Quartile | 1.55 | 1.91 | 1.00 | 1.13 |
| Maximum | 2.30 | 2.31 | 1.89 | 1.75 |
| Number of | 10 | 10 | 10 | 10 |
| Mice | | | | |

TABLE 27
Fold Change in Tumor Volume from Day 12-16

Percentage of mice in each group

| Fold | IM ASC | Control |
|---|---|---|
| Change | (Groups 4/6) | (Groups 1/2) |
| 0-0.5 | 0 | 5 |
| 0.5-1 | 65 | 25 |
| 1-1.5 | 25 | 25 |
| 1.5-2 | 10 | 30 |
| 2-2.5 | 0 | 15 |

TABLE 28
Fold Change in Tumor Volume from Day 9-28. Note the
lack of control mice with fold change of 0 or 0-1, in
contrast to the 8% ASC-treated mice in these groups

| | Percentage of mice in each group | | | |
|---|---|---|---|---|
| Fold | Control Mice | All ASC | All IM ASC | All IV ASC |
| Change | (Groups 1-3) | (Groups 4-7) | (Groups 4/6) | (Groups 5/7) |
| 0 | 0 | 3 | 0 | 6 |
| 0-1 | 0 | 5 | 10 | 0 |
| 1-2 | 13 | 14 | 15 | 12 |
| 2-3 | 30 | 27 | 20 | 35 |
| 3-4 | 13 | 19 | 25 | 12 |
| 4-5 | 30 | 16 | 10 | 24 |
| 5-6 | 3 | 5 | 10 | 0 |
| 6-7 | 7 | 5 | 10 | 0 |
| 7-8 | 0 | 3 | 0 | 6 |
| 8-9 | 3 | 3 | 0 | 6 |

TABLE 29
Fold Change in Tumor Volume from Day 9-28. Note the increased
number of ASC-treated mice with a fold change of <1.5 vs. the control mice

| | Percentage of mice in each group | | | | | |
|---|---|---|---|---|---|---|
| Fold | All ASC | IM ASC | IV ASC | All Control | IM Controls | IV Controls |
| Change | (Gr. 4-7) | (Gr. 4/6) | (Gr. 5/7) | (Gr. 1-3) | (Gr. 1/2) | (Gr. 1/3) |
| 0 | 3 | 0 | 6 | 0 | 0 | 0 |
| 0-0.5 | 0 | 0 | 0 | 3 | 5 | 5 |
| 0.5-1 | 32 | 35 | 29 | 23 | 30 | 15 |
| 1-1.5 | 41 | 35 | 47 | 33 | 15 | 45 |
| 1.5-2 | 11 | 20 | 0 | 23 | 25 | 20 |
| 2-2.5 | 11 | 10 | 12 | 13 | 20 | 10 |
| 2.5-3 | 3 | 0 | 6 | 3 | 5 | 5 |

These data confirm that ASC inhibit tumor growth in vivo, even in a very rapidly-growing tumor model. ASC were also shown to inhibit tumor growth in an additional in vivo tumor implantation model, as described in International Patent Application Publication No. WO 2017/141181 to Zami Aberman et al, which is hereby incorporated by reference in its entirety.

### Example 17

### Effect of ASC CM on Replication and Survival of Primary Tumor Cells

ASC CM is incubated with primary cancer cells in tissue culture dishes, and growth is assayed as described in Example 8. Growth inhibition serves to confirm the relevance of the previous findings in various tumors.

### Example 18

### Effect of ASC on Ectopic Tumors from Primary Tumor Cells

The effect of ASC CM on growth of ectopic tumors from primary tumor cells is determined. The protocol is similar to Example 16, except that primary tumor cells are used to generate the ectopic tumors. Growth inhibition serves to confirm the relevance of the previous findings in various tumors.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications, and Uniprot and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References are Cited in the Text

Barretina, J., et al. (2012). “The Cancer Cell Line Encyclopedia enables predictive modelling of anticancer drug sensitivity.” Nature 483(7391): 603-607.
Croft, D., et al. (2014). “The Reactome pathway knowledgebase.” Nucleic Acids Res 42(Database issue): D472-477.
Dominici et al. Minimal criteria for defining multipotent mesenchymal stromal cells. The International Society for Cellular Therapy position statement. Cytotherapy. 2006; 8(4):315-7.
Forbes, S. A., et al. (2015). “COSMIC: exploring the world's knowledge of somatic mutations in human cancer.” Nucleic Acids Research 43(D1): D805-D811
Friedrich J et al, Spheroid-based drug screen: considerations and practical approach. Nature Protocols 4(3): 309-324, 2009.
Hyman D M et al, Precision medicine at Memorial Sloan Kettering Cancer Center: clinical next-generation sequencing enabling next-generation targeted therapy trials. Drug Discov Today. 2015 December; 20(12):1422-8.
Ivascu A et al. Rapid generation of single-tumor spheroids for highthroughput cell function and toxicity analysis. J. Biomol. Screen 11: 922-932 (2006).
James M A et al, A novel, soluble compound, C25, sensitizes to TRAIL-induced apoptosis through upregulation of DR5 expression. Anticancer Drugs. 2015 June; 26(5):518-30.
Kobayashi K et al, Cytotoxic effects of benzbromarone and its 1′-hydroxy metabolite in human hepatocarcinoma FLC4 cells cultured on micro-space cell culture plates. Drug Metab Pharmacokinet. 2013; 28(3):265-8.
Korff T et al. Integration of endothelial cells in multicellular spheroids prevents apoptosis and induces differentiation. J. Cell Biol. 143: 1341-1352 (1998).
Nakamura et al., Evaluation of drug toxicity with hepatocytes cultured in a micro-space cell culture system. J Biosci Bioeng. 2011 January; 111(1):78-84.
Neve et al. “A Collection of Breast Cancer Cell Lines for the Study of Functionally Distinct Cancer Subtypes.” Cancer cell 10.6 (2006): 515-527
Oh W K, Neoadjuvant therapy before radical prostatectomy in high-risk localized prostate cancer: defining appropriate endpoints. Urol Oncol. 2003 May-June; 21(3):229-34.
Perche F et al, Cancer cell spheroids as a model to evaluate chemotherapy protocols. Cancer Biology & Therapy 13:12, 1205-1213, 2012.
Phung Y T et al, Rapid Generation of In Vitro Multicellular Spheroids for the Study of Monoclonal Antibody Therapy, J Canc 2: 507-514, 2011.
Rahman et al. “TRAIL Induces Apoptosis in Triple-Negative Breast Cancer Cells with a Mesenchymal Phenotype.” Breast cancer research and treatment 113.2 (2009): 217-230.
Ramsey S D et al, Integrating comparative effectiveness design elements and endpoints into a phase III, randomized clinical trial (SWOG S1007) evaluating oncotypeDX-guided management for women with breast cancer involving lymph nodes. Contemp Clin Trials. 2013 January; 34(1):1-9.
Reich, M., et al. (2006). “GenePattern 2.0.” Nat Genet 38(5): 500-501.
Rocha N S et al, (2002) Effects of fasting and intermittent fasting on rat hepatocarcinogeneis induced by diethylnitrosamine. Teratog Carcinog Mutagen. 22(2): 129-138.
Roux S et al, CD4⁺CD25⁺ Tregs control the TRAIL-dependent cytotoxicity of tumor-infiltrating DCs in rodent models of colon cancer. J Clin Invest. 2008 November; 118(11):3751-61.
Ruggeri B A et al, Animal models of disease: pre-clinical animal models of cancer and their applications and utility in drug discovery. Biochem Pharmacol. 2014 Jan. 1; 87(1):150-61.
Vijai J et al, A genome-wide association study of marginal zone lymphoma shows association to the HLA region. Nat Commun. 2015 Jan. 8; 6:5751.
Walker J D et al, Oncolytic herpes simplex virus 1 encoding 15-prostaglandin dehydrogenase mitigates immune suppression and reduces ectopic primary and metastatic breast cancer in mice. J Virol. 2011 July; 85(14):7363-71.

1. A method of determining the susceptibility of a tumor or neoplastic cell to treatment with adherent stromal cells (ASC), the method comprising testing said tumor or neoplastic cell for an ASC treatment informative mutation in an ASC-susceptibility gene selected from:
a. an ASC sensitivity gene, wherein the presence of the ASC treatment informative mutation indicates that the tumor or neoplastic cell will be responsive to treatment with ASC; and
b. an ASC resistance gene, wherein the presence of the ASC treatment informative mutation indicates that the tumor or neoplastic cell will be non-responsive to treatment with ASC.

2. (canceled)

3. A method for evaluating a subject having a tumor, the method comprising:
a. obtaining, from cells of the subject, nucleic acids that comprise one or more sequences of one or more ASC-susceptibility genes selected from:
i. an ASC-sensitivity gene, and
ii. an ASC resistance gene; and
b. performing a sequencing procedure to detect an ASC treatment informative mutation in the one or more sequences of the one or more genes,
wherein:
for an ASC-sensitivity gene, the presence of the ASC treatment informative mutation indicates that the subject will be responsive to treatment with ASC; and
for an ASC-resistance gene, the presence of the ASC treatment informative mutation indicates that the subject will be non-responsive to treatment with ASC.

4-5. (canceled)

6. The method of claim 3, wherein said sequencing procedure is selected from Illumina sequencing, Roche 454 sequencing, Ion torrent sequencing, Ion Proton™ sequencing, and Supported Oligo Ligation Detection (SOLiD) sequencing.

7. An article of manufacture for determining the susceptibility of a tumor or neoplastic cell to treatment with adherent stromal cells (ASC), the article comprising a means of testing said tumor or neoplastic cell for a mutation in an ASC-susceptibility gene selected from:
a. an ASC-sensitivity gene, wherein the presence of a mutation indicates that the tumor will be responsive to treatment with ASC; and
b. an ASC-resistance gene, wherein the presence of a mutation indicates that the tumor will be non-responsive to treatment with ASC.

8-9. (canceled)

10. The article of claim 7, wherein said means comprises hybridization.

11. The method of claim 1, wherein said gene is an ASC resistance gene.

12. (canceled)

13. The method of claim 1, wherein said gene is an ASC-sensitivity gene.

14. (canceled)

15. The method of claim 13, further comprising testing said tumor or neoplastic cell for an ASC treatment informative mutation in an ASC resistance gene.

16-20. (canceled)

21. The method of claim 3, wherein said ASC have been obtained from a three-dimensional (3D) culture.

22. (canceled)

23. The method of claim 21, whereby one or more pro-inflammatory cytokines is added to an incubation medium of said 3D culture.

24. The method of claim 23, wherein said 3D culture comprises: (a) incubating ASC in a 3D culture apparatus in a first growth medium, wherein no inflammatory cytokines have been added to said first growth medium; and (b) subsequently incubating said ASC in a 3D culture apparatus in a second growth medium, wherein one or more pro-inflammatory cytokines have been added to said second growth medium.

25-27. (canceled)

28. The method of claim 21, wherein said 3D culture is performed in an apparatus that comprises a 3D bioreactor.

29. The method of claim 28, wherein said 3D culture is performed in an apparatus that comprises a synthetic adherent material, wherein said synthetic adherent material is selected from the group consisting of a polyester, a polypropylene, a polyalkylene, a poly fluoro-chloro-ethylene, a polyvinyl chloride, a polystyrene, a polysulfone, a cellulose acetate, a glass fiber, and an inert metal fiber.

30. (canceled)

31. The method of claim 28, wherein said 3D culture apparatus comprises microcarriers.

32. The method of claim 31, wherein said microcarriers are packed in said 3D culture apparatus.

33. The method of claim 21, further comprising the subsequent step of harvesting said ASC by removing said ASC from an apparatus wherein said 3D culture was performed.

34. (canceled)

35. The method of claim 3, wherein said ASC originate from placenta tissue.

36-38. (canceled)

39. The method of claim 3, wherein said ASC originate from adipose tissue.

40-41. (canceled)

42. The method of claim 3, wherein said tumor or cancer is selected from non-Hodgkin lymphoma, colorectal cancer, malignant melanoma, thyroid carcinoma, non-small cell lung carcinoma, and lung adenocarcinoma.

43. The method of claim 3, wherein said tumor or cancer is selected from: renal cell carcinoma, melanoma, breast carcinoma, hepatocellular carcinoma, colorectal adenocarcinoma, breast adenocarcinoma, lung adenocarcinoma, large cell lung carcinoma, or rhabdomyosarcoma.

44-46. (canceled)
